(19) United States
(12) Patent Application Publication
(10) Pub. No.: US 2009/0186024 A1
(43) Pub. Date: Jul. 23, 2009

(54) Gene Expression Signatures for Oncogenic Pathway Deregulation

(76) Inventors: Joseph R. Nevins, Chapel Hill, NC (US); Andrea H. Bild, Salt Lake City, UT (US); Guang Yao, Raleigh, NC (US); Jeffrey T. Chang, Durham, NC (US); Quanli Wang, Durham, NC (US); Anil Potti, Chapel Hill, NC (US); David Harpole, Durham, NC (US); Johnathan M. Lancaster, Tampa, FL (US); Andrew Berchuck, Chapel Hill, NC (US); John A. Olson, Chapel Hill, NC (US); Jeffrey R. Marks, Hillsborough, NC (US); Mike West, Durham, NC (US); Holly Dressman, Durham, NC (US)

Correspondence Address: MICHAEL BEST & FRIEDRICH LLP, 100 E WISCONSIN AVENUE, Suite 3300, MILWAUKEE, WI, 53202, US

(21) Appl. No.: 11/920,377

(22) PCT Filed: May 15, 2006
(86) PCT No.: PCT/US2006/018827

§ 371 Date: Apr. 3, 2009

Related U.S. Application Data
(60) Provisional application No. 60/680,490, filed on May 13, 2005.

(51) Int. Cl. A61K 39/395; C12Q 1/68; C40B 30/00; A61K 31/4709

(52) U.S. Cl. 424/134.1; 435/6; 506/7; 514/314

(57) ABSTRACT

The disclosure relates to identifying deregulated pathways in cancer. In certain embodiments, the methods of the disclosure can be used to evaluate therapeutic agents for the treatment of cancer.

### CROSS-REFERENCE TO RELATED APPLICATIONS

This application claims the benefit of U.S. Provisional Patent Application 60/680,490, filed May 13, 2005, the entirety of which is incorporated herein by this reference.

### STATEMENT REGARDING FEDERALLY-SPONSORED RESEARCH OR DEVELOPMENT

The invention described herein was supported, in whole or in part, by Federal Grant No R01-CA104663. The U.S. Government has certain rights in the invention.

### FIELD OF THE INVENTION

The field of this invention is cancer diagnosis and treatment.

### BACKGROUND OF THE INVENTION

Cancer is considered to be a serious and pervasive disease. The National Cancer Institute has estimated that in the United States alone, 1 in 3 people will be afflicted with cancer during their lifetime. Moreover approximately 50% to 60% of people contracting cancer will eventually die from the disease. Lung cancer is one of the most common cancers with an estimated 172,000 new cases projected for 2003 and 157,000 deaths (Jemal et al., 2003, CA Cancer J. Clin., 53, 5-26). Lung carcinomas are typically classified as either small-cell lung carcinomas (SCLC) or non-small cell lung carcinomas (NSCLC). SCLC comprises about 20% of all lung cancers with NSCLC comprising the remaining approximately 80%. NSCLC is further divided into adenocarcinoma (AC) (about 30-35% of all cases), squamous cell carcinoma (SCC) (about 30% of all cases) and large cell carcinoma (LCC) (about 10% of all cases). Additional NSCLC subtypes, not as clearly defined in the literature, include adenosquamous cell carcinoma (ASCC), and bronchioalveolar carcinoma (BAC).

Lung cancer is the leading cause of cancer deaths worldwide, and more specifically non-small cell lung cancer accounts for approximately 80% of all disease cases (Cancer Facts and Figures, 2002, American Cancer Society, Atlanta, p. 11.). There are four major types of non-small cell lung cancer, including adenocarcinoma, squamous cell carcinoma, bronchioalveolar carcinoma, and large cell carcinoma. Adenocarcinoma and squamous cell carcinoma are the most common types of NSCLC based on cellular morphology (Travis et al., 1996, Lung Cancer Principles and Practice, Lippincott-Raven, New York, pps. 361-395). Adenocarcinomas are characterized by a more peripheral location in the lung and often have a mutation in the I-ras oncogene (Gazdar et al., 1994, Anticancer Res. 14:261-267). Squamous cell carcinomas are typically more centrally located and frequently carry p53 gene mutations (Niklinska et al., 2001, Folia Histochem. Cytobiol. 39:147-148).

One particularly prevalent form of cancer, especially among women, is breast cancer. The incidence of breast cancer, a leading cause of death in women, has been gradually increasing in the United States over the last thirty years. In 1997, it was estimated that 181,000 new cases were reported in the U.S., and that 44,000 people would die of breast cancer (Parker et al, 1997, CA Cancer J. Clin. 47:5-27; Chu et al, 1996, J. Nat. Cancer Inst. 88:1571-1579).

Another prevalent form of cancer is ovarian cancer. In 2005, more than 22,000 American women were diagnosed with ovarian cancer and 16,000 women died from the disease. The five-year relative survival rate for stage III and IV disease is 31%, and the five-year relative survival rate for stage I is 95%. Early diagnosis should lower the fatality rate. Unfortunately, early diagnosis is difficult because of the physically inaccessible location of the ovaries, the lack of specific symptoms in early disease, and the limited understanding of ovarian oncogenesis. Screening tests for ovarian cancer need high sensitivity and specificity to be useful because of the low prevalence of undiagnosed ovarian cancer. Because currently available screening tests do not achieve high levels of sensitivity and specificity, screening is not recommended for the general population. The theoretical advantage of screening is much higher for women at high risk (such as those with a strong family history of ovarian cancer and those with BRCA 1 or BRCA 2 mutations). However, even for women at high risk, no prospective studies have shown benefits of screening. The public health challenge is that 90% of ovarian cancer occurs in women who are not in an identifiable high-risk group, and most women are diagnosed with advanced-stage disease. Currently available tests (CA-125, transvaginal ultrasound, or a combination of both) lack the sensitivity and specificity to be useful in screening the general population (Fields and Chevlen, Clin J Oncol Nurs. 2006 February; 10(1):77-81).

Genomic information, in the form of gene expression signatures, has an established capacity to define clinically relevant risk factors in disease prognosis. Recent studies have generated such signatures related to lymph node metastasis and disease recurrence in breast cancer (See West, M. et al. Predicting the clinical status of human breast cancer by using gene expression profiles. Proc. Natl. Acad. Sci., USA 98, 11462-11467 (2001); Spang, R. et al. Prediction and uncertainty in the analysis of gene expression profiles. In Silico Biol. 2, 0033 (2002); van'T Veer, L. J. et al. Gene expression profiling predicts clinical outcome of breast cancer. Nature 415, 530-536 (2002); van de Vijver, M. J. et al. A gene-expression signature as a predictor of survival in breast cancer. N. Engl. J. Med. 347, 1999-2009 (2002); Huang, E. et al. Gene expression predictors of breast cancer outcomes. Lancet in press, (2003)) as well as in other cancers (See Pomeroy, S. L. et al. Prediction of central nervous system embryonal tumour outcome based on gene expression. Nature 415, 436-442 (2002); Alizadeh, A. A. et al. Distinct types of diffuse large B-cell lymphoma identified by gene expression profiling. Nature 403, 503-511 (2000); Rosenwald, A. et al. The use of molecular profiling to predict survival after chemotherapy for diffuse large-B-cell lymphoma; Bhattacharjee, A. et al. Classification of human lung carcinomas by mRNA expression profiling reveals distinct adenocarcinoma subclasses. Proc. Natl. Acad. Sci. USA 98, 13790-13795 (2001); Ramaswamy, S. et al. Multiclass cancer diagnosis using tumor gene expression signatures. Proc. Nat'l. Acad. Sci. 98, 15149-15154 (2001); Golub, T. R. et al. Molecular classification of cancer: class discovery and class prediction by gene expression monitoring. Science 286, 531-537 (1999); Shipp, M. A. et al. Diffuse large B-cell lymphoma outcome prediction by gene expression profiling and supervised machine learning. Nat. Med. 8, 68-74 (2002); Yeoh, E.-J. et al. Classification, subtype discovery, and prediction of outcome in pediatric acute lymphoblastic leukemia by gene expression profiling. Cancer Cell 1, 133-143 (2002)) and non-cancer disease contexts. In spite of considerable research into therapies, these and other cancers remain difficult to diagnose and treat effectively. Accordingly, there is a need in the art for improved methods for classifying and treating such cancers.

### SUMMARY OF THE INVENTION

In certain aspects, the disclosure provides methods of estimating or predicting the efficacy of a therapeutic agent in treating a disorder in a subject, wherein the therapeutic agent regulates a pathway. One aspect provides a method comprising determining the expression levels of multiple genes in a sample from a subject; and detecting the presence of pathway deregulation by comparing the expression levels of the genes to a reference profile indicative of pathway deregulation, wherein the presence of pathway deregulation indicates that the therapeutic agent is estimated to be effective in treating the disorder in the subject. In certain aspects, the disclosure provides methods of estimating or predicting the efficacy of two or more therapeutic agents in treating a disorder in a subject, wherein the therapeutic agents each regulates a different pathway. One aspect provides a method comprising determining the expression levels of multiple genes in a sample from a subject; and detecting the presence of pathway deregulation in each different pathway by comparing the expression levels of the genes to one or more reference profiles indicative of pathway deregulation, wherein the presence of pathway deregulation in the different pathways indicates that the therapeutic agent is estimated to be effective in treating the disorder in the subject.

In certain aspects, the disclosure provides the methods described, wherein said sample is diseased tissue. In certain embodiments, the sample is a tumor sample. In certain embodiments, the tumor is selected from a breast tumor, an ovarian tumor, and a lung tumor. In certain embodiments, the therapeutic agents are selected from a farnesyl transferase inhibitor, a farnesylthiosalicylic acid, and a Src inhibitor. In certain embodiments, the pathway is selected from RAS, SRC, MYC, E2F, and β-catenin pathways. In certain embodiments, the measure of efficacy of a therapeutic agent is selected from the group consisting of disease-specific survival, disease-free survival, tumor recurrence, therapeutic response, tumor remission, and metastasis inhibition.

In certain aspects, the disclosure provides the methods described, wherein detecting the presence of pathway deregulation by comparing the expression levels of the genes to a reference profile indicative of pathway deregulation, comprises detecting the presence of pathway deregulation in the different pathways by using supervised classification methods of analysis. In certain embodiments, detecting the presence of pathway deregulation by comparing the expression levels of the genes to a reference profile indicative of pathway deregulation comprises comparing samples with known deregulated pathways to controls to generate signatures; and comparing the expression profile from the subject sample to the said signatures to indicate pathway deregulation.

In certain aspects, the disclosure provides methods of determining or helping to determine the deregulation status of multiple pathways in a tumor sample. One aspect provides a method comprising: obtaining an expression profile for said sample; and comparing said obtained expression profile to a reference profile to determine deregulation status of said pathways. In certain embodiments, the deregulation status of the pathways is hyperactivation. In certain embodiments, the deregulation status of the pathways is hypoactivation.

In certain aspects, the disclosure provides methods of estimating or predicting the efficacy of a therapeutic agent in treating cancer cells, wherein the therapeutic agent regulates a pathway. One aspect provides a method comprising: determining the expression levels of multiple genes in a sample from a subject; and detecting the presence of pathway deregulation by comparing the expression levels of the genes to a reference profile indicative of pathway deregulation, wherein the presence of pathway deregulation indicates that the therapeutic agent is estimated to be effective in treating the cancer cells. In certain aspects, the disclosure provides methods of using pathway signatures to analyze a large collection of human tumor samples to obtain profiles of the status of multiple pathways in said tumors. One aspect provides a method comprising: determining the expression levels of multiple genes in a sample from a subject; and identifying patterns of pathway deregulation by comparison of the expression profiles with a reference profile. In certain aspects, the disclosure provides methods of treating or helping to treat a subject afflicted with cancer. One aspect provides a method comprising: identifying a pathway that is deregulated in a tumor sample from a subject; selecting a therapeutic agent known to modulate the activity level of the pathway; and administering to the subject an effective amount of the therapeutic agent, thereby treating the subject afflicted with cancer. In certain aspects, the disclosure provides methods of treating or helping to treat a subject afflicted with cancer. One aspect provides a method comprising: identifying two or more pathways that are deregulated in a tumor sample from a subject; selecting a therapeutic agent known to modulate the activity level of each pathway; and administering to the subject an effective amount of the therapeutic agents, thereby treating the subject afflicted with cancer.

In certain aspects, the disclosure provides methods of treating or helping to treat a subject afflicted with cancer, wherein a therapeutic agent is a combination of two or more therapeutic agents. In certain aspects, the disclosure provides a method of treating a subject afflicted with cancer, wherein identifying a pathway that is deregulated in the tumor sample comprises: obtaining an expression profile from said sample; and comparing said obtained expression profile to a reference profile to determine the deregulation status of multiple pathways for said subject.

In certain aspects, the disclosure provides methods of reducing side effects from the administration of two or more agents to a subject afflicted with cancer. One aspect provides a method comprising: determining a cancer subtype for said subject by: obtaining an expression profile from a sample from said subject; and comparing said obtained expression profile to a reference profile to determine the deregulation status of multiple pathways for said subject; determining ineffective treatment protocols based on said determined cancer subtype; reducing side effects by not treating said subject with said ineffective treatment protocols. In certain embodiments, ineffective treatment protocols are determined by comparing the deregulated pathways of the cancer to the pathway targeted by the treatment protocol. In some embodiments, a treatment may be determined to be ineffective if the targeted pathway is not deregulated. In other embodiments, a treatment may be determined to be ineffective if the targeted pathway is deregulated. In preferred embodiments, ineffective treatments with potential harmful side effects are avoided. In certain aspects, the disclosure provides methods of generating an expression signature for a deregulated pathway. One aspect provides a method comprising: overexpressing an oncogene in a cell line to deregulate a pathway; determining an expression profile of multiple genes in the cell line; and comparing said obtained expression profile to a reference profile to determine an expression signature for a deregulated pathway. In certain embodiments, overexpressing an oncogene comprises transfecting the cell line with the oncogene. In certain embodiments, the expression profile is obtained by the use of microarrays. In certain embodiments, the expression profile comprises ten or more genes, 20 or more genes, 50 or more genes.

In certain aspects, the disclosure provides methods of generating an expression signature for a deregulated pathway. One aspect provides a method comprising: underexpressing a tumor suppressor in a cell line to deregulate a pathway; determining an expression profile of multiple genes in the cell line; and comparing said obtained expression profile to a reference profile to determine an expression signature for a deregulated pathway. In certain embodiments, underexpressing a tumor suppressor comprises targeted gene knockdown or knockout of the tumor suppressor in a cell line. In certain embodiments, the expression profile is obtained by the use of a microarray. In certain embodiments, the expression profile comprises ten or more genes, 20 or more genes, 50 or more genes. In a preferred embodiment, the deregulated pathway of the disclosure is an oncogenic pathway. In a preferred embodiment the deregulated pathway is a RAS pathway. In a preferred embodiment the deregulated pathway is the Myc pathway. In a preferred embodiment the deregulated pathway is the β-catenin pathway. In a preferred embodiment the deregulated pathway is the E2F3 pathway. In a preferred embodiment the deregulated pathway is the Src pathway. In some embodiments, the deregulated pathways are all or a combination of these pathways.

The methods described in the invention are useful for the integration of genomic information into prognostic models that can be applied in a clinical setting to improve the accuracy of treatment decisions as well as the development of new treatment and drug regiments for the treatment of disease.

### BRIEF DESCRIPTION OF THE FIGURES

FIGS. 1A-1B show gene expression patterns that predict oncogenic pathway deregulation. A. Image intensity display of expression levels of the genes most highly weighted in the predictor differentiating GFP expressing control cells from cells expressing the indicated oncogenic activity. Expression levels are standardized to zero mean and unit variance across samples, displayed with genes as rows and samples as columns, and color coded to indicate high/low expression levels in red/blue. B. Scatter plot depicting the classification of samples based on the first three principal components (expression patterns) derived from each signature, as shown in panel A. The gene expression values for each signature were extracted from all experimental samples and mean centered, then single value decomposition (SVD) analysis was applied across all samples. Color coding for samples is Myc (blue), Ras (green), E2F3 (purple), Src (yellow), β-catenin (red). Samples representing the specific pathway being examined are circled.

FIGS. 2A-2C show validation of pathway predictions in tumors. A. Mouse mammary tumors derived from mice transgenic for the MMTV-MYC (5 samples), MMTV-HRAS (3 samples) or MMTV-NEU (7 samples) oncogenes, tumors dependent on loss of Rb (6 samples), or 7 samples of normal mammary tissue was used to verify accuracy and specificity of our signatures. The predicted probability of Myc, E2F3, and Ras activity in mouse tumors were sorted from low (blue) to high (red), and displayed as a colorbar. B. Prediction of pathway status in mouse lung cancer model. A set of previously published mouse Affymetrix expression data comparing normal and tumor lung tissue with spontaneous activating kRAS mutations¹⁴ were used to validate the predictive capacity of the Ras pathway signature. The predicted probability of Ras activity in the normal and tumor tissue was sorted from low to high, and displayed as a colorbar. C. Relationship of Ras pathway status in NSCLC samples to cell type of tumor origin. The corresponding tumor cell type is indicated as either squamous (S) or adenocarcinoma (A). Ras mutation status indicated by (*).

FIGS. 3A-3C show patterns of pathway deregulation in human cancers. A. Left panel. Hierarchical clustering of predictions of pathway deregulation in samples of human lung tumors. Prediction of Ras, Myc, E2F3, β-catenin, and Src pathway status for each tumor sample was independently determined using supervised binary regression analysis as described. Patterns in the tumor pathway predictions were identified by hierarchical clustering, and separate clusters are indicated by colored dendograms. Right panel. Kaplan-Meier survival analysis for lung cancer patients based on pathway clusters. Patient clusters with correlative pathway deregulation shown in left panel correspond to clusters comprising each independent survival curve. Black tick marks represent censored patients. B. Breast cancer. Same as in panel A. C. Ovarian cancer. Same as in panel A.

FIGS. 4A-4B show pathway deregulation in breast cancer cell lines predicts drug sensitivity. A. Pathway predictions in breast cancer cell lines. The results plotted show images of the predicted probability of pathway activation (red indicates high probability, blue indicates low probability). B. Sensitivity to pathway-specific drugs. Left panel. Cells were treated with 3.75 μM of farnesyltransferase inhibitor (L-744,832) for 96 hrs. Proliferation was assayed using a standard MTS tetrazolium colorimetric method. The degree of proliferation inhibition was plotted as a function of probability of Ras pathway activation as determined in panel A. Middle panel. Same as in left panel but using farnesylthiosalicylic acid (200 μM). Right panel. Same as in left panel but using the Src pathway inhibitor SU6656 (1.5 μM), and with the degree of proliferation inhibition plotted as a function of Src pathway activation.

FIG. 5 shows biochemical assays of pathway activation. HMEC were infected with either control GFP or a specific oncogene following 36 hours of serum starvation. After 18 hours, cells were collected, and Western Blotting analysis was performed as described in Materials and Methods to measure the expression of the encoded protein or downstream targets of the pathway.

FIG. 6 shows gene expression patterns that predict oncogenic pathway deregulation. Leave-one-out cross-validation predicted classification probabilities for each individual sample. Pathway status for each experimental sample was predicted using a model generated independently of that sample. These predictions are based on the screened subset of discriminatory genes that comprise each signature model. The values on the horizontal axis are estimates of the overall signature scores in the regression analysis, and the corresponding values on the vertical axis are estimated classification probabilities. The GFP control samples are shown in blue and the oncogenic pathway samples in red.

FIG. 7 shows validation of pathway predictions in tumors. Relationship of Ras pathway status in NSCLC samples to cell type of tumor origin. Prediction of Ras status in tumors is presented as a colorbar, where samples were sorted from low (blue) to high (red) activity. The corresponding tumor cell type is indicated as either squamous (S) or adenocarcinoma (A). Ras mutation status indicated by (*).

FIGS. 8A-8C show Kaplan-Meier survival analysis for cancer patients based on individual pathway predictions for the tumor dataset. A. Lung cancer. Patients were classified as low or high probability of activation of the indicated pathway based on expression signatures (low probability<50%; high probability≧50%). Kaplan-Meier survival curves were then generated for these two groups. B. Breast cancer. Same as in panel A. C. Ovarian cancer. Same as in panel A.

FIG. 9 shows assays for pathway activities in breast cancer cell lines. Activity of E2F3, Myc, Src, β-catenin, and H-Ras pathways.

FIG. 10 shows the relationship of drug sensitivity to predictions of untargeted pathways. The degree of proliferation inhibition was plotted as a function of pathway prediction not specific to the drug treatment.

### DETAILED DESCRIPTION OF THE INVENTION

### Overview

The development of an oncogenic state is a complex process involving the accumulation of multiple independent mutations that lead to deregulation of cell signaling pathways that are central to control cell growth and cell fate¹⁻³. The ability to define cancer subtypes, recurrence of disease, and response to specific therapies using DNA microarray-based gene expression signatures has been demonstrated in multiple studies⁴. The invention provides novel methods by which gene expression signatures can be identified that reflect the activation status of several oncogenic pathways. When evaluated in several large collections of human cancers, these gene expression signatures identify patterns of pathway deregulation in tumors, and clinically relevant associations with disease outcomes. Combining signature-based predictions across several pathways identifies coordinated patterns of pathway deregulation that distinguish between specific cancers and tumor sub-types. Clustering tumors based on pathway signatures further defines prognosis in respective patient subsets, demonstrating that patterns of oncogenic pathway deregulation underlie the development of the oncogenic phenotype and reflect the biology and outcome of specific cancers. Importantly, predictions of pathway deregulation in cancer cell lines are shown to also predict the sensitivity to therapeutic agents that target components of the pathway. Identifying functional characteristics of tumors has the potential to link pathway deregulation with therapeutics that target components of the pathway, and leads to the immediate opportunity to make use of these oncogenic pathway signatures to guide the use of targeted therapeutics.

### DEFINITIONS

Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal.

The term “expression vector” and equivalent terms are used herein to mean a vector which is capable of inducing the expression of DNA that has been cloned into it after transformation into a host cell. The cloned DNA is usually placed under the control of (i.e., operably linked to) certain regulatory sequences such a promoters or enhancers. Promoters sequences may be constitutive, inducible or repressible.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The term “recombinant” is used herein to mean any nucleic acid comprising sequences which are not adjacent in nature. A recombinant nucleic acid may be generated in vitro, for example by using the methods of molecular biology, or in vivo, for example by insertion of a nucleic acid at a novel chromosomal location by homologous or non-homologous recombination.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “prophylactic” or “therapeutic” treatment refers to administration to the subject of one or more of the subject compositions. If it is administered prior to clinical manifestation of the unwanted condition (e.g., cancer or the metastasis of cancer) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain cell lines of the present invention may be administered in a sufficient amount to produce a reasonable benefit/risk ratio applicable to such treatment.

The term “effective amount” refers to the amount of a therapeutic reagent that when administered to a subject by an appropriate dose and regimen produces the desired result.

The term “subject in need of treatment for a disorder” is a subject diagnosed with that disorder or suspected of having that disorder.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies.

The term “antineoplastic agent” is used herein to refer to agents that have the functional property of inhibiting a development or progression of a neoplasm or neoplastic cell growth in a human, particularly a malignant (cancerous) lesion, such as a carcinoma, sarcoma, lymphoma, or leukemia.

The terms “overexpressed” or “underexpressed” typically relate to expression of a nucleic acid sequence or protein in a cancer cell at a higher or lower level, respectively, than that level typically observed in a non-tumor cell (i.e., normal control). In preferred embodiments, the level of expression of a nucleic acid or a protein that is overexpressed in the cancer cell is at least 10%, 20%, 40%, 60%, 80%, 100%, 200%, 400%, 500%, 750%, 1,000%, 2,000%, 5,000%, or 10,000% greater in the cancer cell relative to a normal control.

The term “sensitive to a drug” or “resistant to a drug” is used herein to refer to the response of a cell when contacted with an agent. A cancer cell is said to be sensitive to a drug when the drug inhibits the cell growth or proliferation of the cell to a greater degree than is expected for an appropriate control, such as an average of other cancer cells that have been matched by suitable criteria, including but not limited to, tissue type, doubling rate or metastatic potential. In some embodiments, greater degree refers to at least 10%, 20%, 30%, 40%, 50%, 60%, 70%, 80%, 90%, 100%, 200%, or 500%. A cancer cell is said to be sensitive to a drug when the drug inhibits the cell growth or proliferation of the cell to a lesser degree than is expected for an appropriate control, such as an average of other cancer cells that have been matched by suitable criteria, including but not limited to, tissue type, doubling rate or metastatic potential. In some embodiments, lesser degree refers to at least 10%, 15%, 20%, 25%, 50% or 100% less.

The phrase “predicting the likelihood of developing” as used herein refers to methods by which the skilled artisan can predict onset of a vascular condition or event in an individual. The term “predicting” does not refer to the ability to predict the outcome with 100% accuracy. Instead, the skilled artisan will understand that the term “predicting” refers to forecast of an increased or a decreased probability that a certain outcome will occur; that is, that an outcome is more likely to occur in an individual with specific deregulated pathways.

As used herein, the term “pathway” is intended to mean a set of system components involved in two or more sequential molecular interactions that result in the production of a product or activity. A pathway can produce a variety of products or activities that can include, for example, intermolecular interactions, changes in expression of a nucleic acid or polypeptide, the formation or dissociation of a complex between two or more molecules, accumulation or destruction of a metabolic product, activation or deactivation of an enzyme or binding activity. Thus, the term “pathway” includes a variety of pathway types, such as, for example, a biochemical pathway, a gene expression pathway and a regulatory pathway. Similarly, a pathway can include a combination of these exemplary pathway types.

The term “deregulated pathway” is used herein to mean a pathway that is either hyperactivated or hypoactivated. A pathway is hyperactivated if it has at least 10%, 20%, 50%, 75%, 100%, 200%, 500%, 1000% greater activity/signaling than the normal pathway. A pathway is hypoactivated if it has at least 10%, 20%, 50%, 75%, 100%, 200%, 500%, 1000% less activity/signaling than the normal pathway. The change in activation status may be due to a mutation of a gene (such as point mutations, deletion, or amplification), changes in transcriptional regulation (such as methylation, phosphorylation, or acetylation changes), or changes in protein regulation (such as translational or post-translational control mechanisms).

The term “oncogenic pathway” is used herein to mean a pathway that when hyperactivated or hypoactivated contributes to cancer initiation or progression. In one embodiment, an oncogenic pathway is one that contains an oncogene or a tumor suppressor gene.

### DESCRIPTION OF THE SPECIFIC EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

### Pathways

In one embodiment, the deregulated pathway is a biochemical pathway. A biochemical pathway can include, for example, enzymatic pathways that result in conversion of one compound to another, such as in metabolism, and signal transduction pathways that result in alterations of enzyme activity, polypeptide structure, and polypeptide functional activity. Specific examples of biochemical pathways include the pathway by which galactose is converted into glucose-6-phosphate and the pathway by which a photon of light received by the photoreceptor rhodopsin results in the production of cyclic AMP. Numerous other biochemical pathways exist and are well known to those skilled in the art.

In some embodiments, the biochemical pathway is a carbohydrate metabolism pathway, which in a specific embodiment is selected from the group consisting of glycolysis/gluconeogenesis, citrate cycle (TCA cycle), pentose phosphate pathway, pentose and glucuronate interconversions, fructose and mannose metabolism, galactose metabolism, Ascorbate and aldarate metabolism, starch and sucrose metabolism, amino sugars metabolism, nucleotide sugars metabolism, pyruvate metabolism, glyoxylate and dicarboxylate metabolism, propionate metabolism, butanoate metabolism, C₅-branched dibasic acid metabolism, inositol metabolism and inositol phosphate metabolism.

In some embodiments, the biochemical pathway is an energy metabolism pathway, which in a specific embodiment is selected from the group consisting of oxidative phosphorylation, ATP synthesis, photosynthesis, carbon fixation, reductive carboxylate cycle (CO₂ fixation), methane metabolism, nitrogen metabolism and sulfur metabolism.

In some embodiments, the biochemical pathway is a lipid metabolism pathway, which in a specific embodiment is selected from the group consisting of fatty acid biosynthesis (path 1), fatty acid biosynthesis (path 2), fatty acid metabolism, synthesis and degradation of ketone bodies, biosynthesis of steroids, bile acid biosynthesis, C21-steroid hormone metabolism, androgen and estrogen metabolism, glycerolipid metabolism, phospholipid degradation, prostaglandin and leukotriene metabolism.

In some embodiments, the biochemical pathway is a nucleotide metabolism pathway, which in a specific embodiment is selected from the group consisting of purine metabolism and pyrimidine metabolism.

In some embodiments, the biochemical pathway is an amino acid metabolism pathway, which in a specific embodiment is selected from the group consisting of glutamate metabolism, alanine and aspartate metabolism, glycine, serine and threonine metabolism, methionine metabolism, cysteine metabolism, valine, leucine and isoleucine degradation, valine, leucine and isoleucine biosynthesis, lysine biosynthesis, lysine degradation, arginine and proline metabolism, histidine metabolism, tyrosine metabolism, phenylalanine metabolism, tryptophan metabolism, phenylalanine, tyrosine and tryptophan biosynthesis, urea cycle, beta-Alanine metabolism, taurine and hypotaurine metabolism, aminophosphonate metabolism, selenoamino acid metabolism, cyanoamino acid metabolism, D-glutamine and D-glutamate metabolism, D-arginine and D-ornithine metabolism, D-alanine metabolism and glutathione metabolism.

In some embodiments, the biochemical pathway is a glycan biosynthesis and metabolism pathway, which in a specific embodiment is selected from the group consisting of N-glycans biosynthesis, N-glycan degradation, O-glycans biosynthesis, chondroitin/heparan sulfate biosynthesis, keratan sulfate biosynthesis, glycosaminoglycan degradation, lipopolysaccharide biosynthesis, clycosylphosphatidylinositol (GPI)-anchor biosynthesis, peptidoglycan biosynthesis, glycosphingolipid metabolism, blood group glycolipid biosynthesis—lactoseries, blood group glycolipid biosynthesis—neo-lactoseries, globoside metabolism and ganglioside biosynthesis.

In some embodiments, the biochemical pathway is a biosynthesis of Polyketides and Nonribosomal Peptides pathway, which in a specific embodiment is selected from the group consisting of Type I polyketide structures, biosynthesis of 12-, 14- and 16-membered macrolides, biosynthesis of ansamycins, polyketide sugar unit biosynthesis, nonribosomal peptide structures, and siderophore group nonribosomal peptide biosynthesis.

In some embodiments, the biochemical pathway is a metabolism of cofactors and vitamins pathway, which in a specific embodiment is selected from the group consisting of Thiamine metabolism, Riboflavin metabolism, Vitamin B6 metabolism, Nicotinate and nicotinamide metabolism, Pantothenate and CoA biosynthesis, Biotin metabolism, Folate biosynthesis, One carbon pool by folate, Retinol metabolism, Porphyrin and chlorophyll metabolism and Ubiquinone biosynthesis.

In some embodiments, the biochemical pathway is a biosynthesis of secondary metabolites pathway, which in a specific embodiment is selected from the group consisting of terpenoid biosynthesis, diterpenoid biosynthesis, monoterpenoid biosynthesis, limonene and pinene degradation, indole and ipecac alkaloid biosynthesis, flavonoids, stilbene and lignin biosynthesis, alkaloid biosynthesis I, alkaloid biosynthesis II, penicillins and cephalosporins biosynthesis, beta-lactam resistance, streptomycin biosynthesis, tetracycline biosynthesis, clavulanic acid biosynthesis and puromycin biosynthesis.

In one embodiment, the deregulated pathway is a gene expression pathway. A gene expression pathway can include, for example, molecules which induce, enhance or repress expression of a particular gene. A gene expression pathway can therefore include polypeptides that function as repressors and transcription factors that bind to specific DNA sequences in a promoter or other regulatory region of the one or more regulated genes. An example of a gene expression pathway is the induction of cell cycle gene expression in response to a growth stimulus.

In one embodiment, the deregulated pathway is a regulatory pathway. A regulatory pathway can include, for example, a pathway that controls a cellular function under a specific condition. A regulatory pathway controls a cellular function by, for example, altering the activity of a system component or the activity of a biochemical, gene expression or other type of pathway. Alterations in activity include, for example, inducing a change in the expression, activity, or physical interactions of a pathway component under a specific condition. Specific examples of regulatory pathways include a pathway that activates a cellular function in response to an environmental stimulus of a biochemical system, such as the inhibition of cell differentiation in response to the presence of a cell growth signal and the activation of galactose import and catalysis in response to the presence of galactose and the absence of repressing sugars. The term “component” when used in reference to a network or pathway is intended to mean a molecular constituent of the biochemical system, network or pathway, such as, for example, a polypeptide, nucleic acid, other macromolecule or other biological molecule.

In one embodiment, the deregulated pathway is a signaling pathway. Signaling pathways include MAPK signaling pathways, Wnt signaling pathways, TGF-beta signaling pathways, toll-like receptor signaling pathways, Jak-STAT signaling pathways, second messenger signaling pathways and phosphatidylinositol signaling pathways.

In one embodiment, the pathway, or the deregulated pathway, contains a tumor suppressor or an oncogene or both. The pathways to which an oncogene or a tumor suppressor gene are assigned are well known in the art, and may be assigned by consulting any of several databases which describe the function of genes and their classification into pathways and/or by consulting the literature (See also Biochemical Pathways: An Atlas of Biochemistry and Molecular Biology. Gerhard Michal (Editor) Wiley, John & Sons, Incorporated, (1998); Biochemistry of Signal Transduction and Regulation, Gerhard Krauss, Wiley, John & Sons, Incorporated, (2003); Signal Transduction. Bastien D. Gomperts, Academic Press, Incorporated (2003)). Databases which may be used include, but are not limited to, http://www.genome.jp/kegg/kegg4.html; Pubmed, OMIM and Entrez at http://www.ncbi.nih.gov; the Swiss-Prot database at http://www.expasy.org/.

In one preferred embodiment, a pathway to which an oncogene or tumor suppressor is assigned is identified using the Biomolecular Interaction Network Database (BIND) at http://www.blueprint.org/bind/, and more preferably at http://www.blueprint.org/bind/search/bindsearch.html (See also Bader G D, Betel D, Hogue C W. (2003) BIND: the Biomolecular Interaction Network Database. Nucleic Acids Res. 31(1):248-50; and Bader G D, Hogue C W. (2003) An automated method for finding molecular complexes in large protein interaction networks. BMC Bioinformatics. 4(1)). One feature of the BIMD database lists the pathways to which a query gene has been assigned, thereby allowing the identification of the pathways to which a gene is assigned. Furthermore, U.S. Patent Publication No. 2003/0100996 describes methods for establishing a pathway database and performing pathway searches which may be used to facilitate the identification of pathways and the classification of genes into pathways.

In certain embodiments, oncogenes that may be used in the methods of the disclosure include but are not limited to: abl, akt-2, alk, aml1, ax1, bcl-2, bcl-3, bcl-6, c-myc, dbl, egfr, erbB, erbB2, ets-1, fms, fos, fps, gip, gli, gsp, hox11, hst, IL-3, int-2, kit, KS3, K-sam, Lbc, lck, lmo-1, lmo-2, L-myc, lyl-1, lyt-10, mas, mdm-2, MLH1, MLM, mos, MSH2, myb, N-myc, ost, pax-5, pim-1, PMS1, PMS2, PRAD-1, raf, N-RAS, K-RAS, H-RAS, ret, rhom-1, rhom-2, ros, ski, sis, Src, tal-1, tal-2, tan-1, Tiam-1, trk. In certain embodiments, tumor suppressors that may be used in the methods of the disclosure include but are not limited to: APC, BRCA1, BRCA2, CDKN2A, DCC, DPC4, SMAD2, MEN1, MTS1, NF1, NF2, p53, PTEN, Rb, TSC1, TSC2, VHL, WRN, WT1.

In certain embodiments, the disclosure relates to identifying deregulated pathways in a tumor sample. In preferred embodiments, the deregulated pathway is an oncogenic pathway. The deregulated pathway of the disclosure may be a known oncogenic pathways known to contribute to cancer (for examples see Hanahan and Weinberg Cell. 2000 Jan. 7; 100(1):57-70.) or a novel one.

In a preferred embodiment, the deregulated pathway is the Ras pathway (see Giehl, Biol Chem. 2005 March; 386(3):193-205). The ras genes give rise to a family of related GTP-binding proteins that exhibit potent transforming potential. Mutational activation of Ras proteins promotes oncogenesis by disturbing a multitude of cellular processes, such as gene expression, cell cycle progression and cell proliferation, as well as cell survival, and cell migration. Ras signalling pathways are well known for their involvement in transformation and tumour progression, especially the Ras effector cascade Raf/MEK/ERK, as well as the phosphatidylinositol 3-kinase/Akt pathway.

In a preferred embodiment, the deregulated pathway is the Myc pathway (see Dang et al., Exp Cell Res. 1999 Nov. 25; 253(1):63-77). The c-myc gene and the expression of the c-Myc protein are frequently altered in human cancers. The c-myc gene encodes the transcription factor c-Myc, which heterodimerizes with a partner protein, termed Max, to regulate gene expression. Max also heterodimerizes with the Mad family of proteins to repress transcription, antagonize c-Myc, and promote cellular differentiation. The constitutive activation of c-myc expression is key to the genesis of many cancers, and hence the understanding of c-Myc function depends on our understanding of its target genes. c-Myc emerges as an oncogenic transcription factor that integrates the cell cycle machinery with cell adhesion, cellular metabolism, and the apoptotic pathways.

In a preferred embodiment, the deregulated pathway is the β-catenin pathway (see Moon, Sci STKE. 2005 Feb. 15; 2005 (271):cm1). Wnts are secreted glycoproteins that act as ligands to stimulate receptor-mediated signal transduction pathways in both vertebrates and invertebrates. Activation of Wnt pathways can modulate cell proliferation, survival, cell behavior, and cell fate in both embryos and adults. The Wnt/beta-catenin pathway is the best understood Wnt signaling pathway, and its core components are highly conserved during evolution, although tissue-specific or species-specific modifiers of the pathway are likely. In the absence of a Wnt signal, cytoplasmic beta-catenin is phosphorylated and degraded in a complex of proteins. Wnt signaling through the Frizzled serpentine receptor and low-density lipoprotein receptor-related protein-5 or -6 (LRP5 or 6) coreceptors activates the cytoplasmic phosphoprotein Dishevelled, which blocks the degradation of beta-catenin. As the amount of beta-catenin rises, it accumulates in the nucleus, where it interacts with specific transcription factors, leading to regulation of target genes. Inappropriate activation of the pathway in response to mutations is linked to a wide range of cancers, including colorectal cancer and melanoma.

In a preferred embodiment, the deregulated pathway is the E2F3 pathway (see Aslanian et al., Genes Dev. 2004 Jun. 15; 18(12):1413-22). Tumor development is dependent upon the inactivation of two key tumor-suppressor networks, p16(Ink4a)-cycD/cdk4-pRB-E2F and p19(Arf)-mdm2-p53, that regulate cellular proliferation and the tumor surveillance response. E2F3 is a key repressor of the p19(Arf)-p53 pathway in normal cells. Consistent with this notion, Arf mutation suppresses the activation of p53 and p21(Cip1) in E2f3-deficient MEFs. Arf loss also rescues the known cell cycle re-entry defect of E2f3(−/−) cells, and this correlates with restoration of appropriate activation of classic E2F-responsive genes. There is a direct role for E2F in the oncogenic activation of Arf.

In a preferred embodiment, the deregulated pathway is the Src pathway (Summy and Gallick, Cancer Metastasis Rev. 2003 December; 22(4):337-58). The Src family of non-receptor protein tyrosine kinases plays critical roles in a variety of cellular signal transduction pathways, regulating such diverse processes as cell division, motility, adhesion, angiogenesis, and survival. Constitutively activated variants of Src family kinases, including the viral oncoproteins v-Src and v-Yes, are capable of inducing malignant transformation of a variety of cell types. Src family kinases, most notably although not exclusively c-Src, are frequently overexpressed and/or aberrantly activated in a variety of epithelial and non-epithelial cancers. Activation is very common in colorectal and breast cancers, and somewhat less frequent in melanomas, ovarian cancer, gastric cancer, head and neck cancers, pancreatic cancer, lung cancer, brain cancers, and blood cancers. Further, the extent of increased Src family activity often correlates with malignant potential and patient survival. Activation of Src family kinases in human cancers may occur through a variety of mechanisms and is frequently a critical event in tumor progression. Exactly how Src family kinases contribute to individual tumors remains to be defined completely, however they appear to be important for multiple aspects of tumor progression, including proliferation, disruption of cell/cell contacts, migration, invasiveness, resistance to apoptosis, and angiogenesis.

### Samples and Cell Lines

In certain embodiments, samples of the disclosure are cells from tumors. In certain embodiments, samples are taken from human tumors. In preferred embodiments, samples are taken from a subject afflicted with cancer. In a most preferred embodiment, the samples are breast, ovarian or lung cancer. In some embodiments, samples may come from cell lines. In certain embodiments, samples may be from a collection of tissues or cell lines. In one embodiment, the samples are ex vivo tumor samples.

In a specific embodiment, the subject according to the methods described herein is afflicted with, is suspected of being afflicted with, is likely to be afflicted with, or has been afflicted with at least one solid tumor or one non solid tumor, including carcinomas, adenocarcinomas and sarcomas. Nonlimiting examples of tumors includes fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, uterine cancer, breast cancer including ductal carcinoma and lobular carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, leukemias, lymphomas, and multiple myelomas.

In certain embodiments, the subtype of the cancer determined by the methods of the invention may be a stage or a grade or a combination there of. Depending upon the extent of a cancer (such as breast cancer), a tumor stage (I, II, III, or IV) is assigned, with stage I disease representing the earliest cancers, and stage IV indicating the most advanced. The stage of a cancer is important because it helps determine the best treatment options and is generally predictive of outcome (prognosis). Some cancers such as prostate cancer are subtyped into grades. Grade 1 (Low Grade or Well Differentiated) cancer cells still look a lot like normal cells. They are usually slow growing. Grade 2 (Intermediate/Moderate Grade or Moderately Differentiated) cancer cells do not look like normal cells. They are growing somewhat faster than normal cells. Grade 3 (High Grade or Poorly Differentiated) cancer cells do not look at all like normal cells. They are fast-growing.

In a preferred embodiment, the subject according to the methods described herein is afflicted with, is suspected of being afflicted with, is likely to be afflicted with, or has been afflicted with breast cancer. In a preferred embodiment, the subject according to the methods described herein is afflicted with, is suspected of being afflicted with, is likely to be afflicted with, or has been afflicted with ovarian cancer. In a preferred embodiment, the subject according to the methods described herein is afflicted with, is suspected of being afflicted with, is likely to be afflicted with, or has been afflicted with lung cancer. In some embodiments the cancer may be non-small cell lung carcinoma (NSCLC).

### Collections of Genes and Metagenes Identified by the Invention

The methods of the invention may be directed to a collection of genes whose expression is correlated with deregulated pathways. In on embodiment, this biological state is a disease state. Such disease states include, but are not limited to cancer, such as breast cancer, ovarian cancer, and lung cancer. Thus, the invention is directed to collections of phenotype determinative genes, as well as methods for using the collection or subparts thereof in various applications. Applications in which the collection finds use, include diagnostic, therapeutic and screening applications. Also reviewed are reagents and kits for use in practicing the subject methods. Finally, a review of various methods of identifying genes whose expression correlates with a given phenotype is provided.

The subject invention provides a collection of phenotype determinative genes. By phenotype determinative genes is meant genes whose expression or lack thereof correlates with a phenotype. Thus, phenotype determinative genes include genes: (a) whose expression is correlated with the phenotype, i.e., are expressed in cells and tissues thereof that have the phenotype, and (b) whose lack of expression is correlated with the phenotype, i.e., are not expressed in cells and tissues thereof that have the phenotype. A cell is a cell with the indicated phenotype if it is obtained from tissue that is determined to display that phenotype through methods known to those skilled in the art.

The invention provides all collections and subsets thereof of phenotype determinative genes as well as metagenes disclosed herewith. The subject collections of phenotype determinative genes may be physical or virtual. Physical collections are those collections that include a population of different nucleic acid molecules, where the phenotype determinative genes are represented in the population, i.e., there are nucleic acid molecules in the population that correspond in sequence to the genomic, or more typically, coding sequence of the phenotype determinative genes in the collection. In many embodiments, the nucleic acid molecules are either substantially identical or identical in sequence to the sense strand of the gene to which they correspond, or are complementary to the sense strand to which they correspond, typically to an extent that allows them to hybridize to their corresponding sense strand under stringent conditions. An example of stringent hybridization conditions is hybridization at 50.degree. C. or higher and 0.1.times.SSC (15 mM sodium chloride/1.5 mM sodium citrate). Another example of stringent hybridization conditions is overnight incubation at 42.degree. C. in a solution: 50% formamide, 5.times.SSC (150 mM NaCl, 15 mM trisodium citrate), 50 mM sodium phosphate (pH7.6), 5.times. Denhardt's solution, 10% dextran sulfate, and 20.mu.g/ml denatured, sheared salmon sperm DNA, followed by washing the filters in 0.1.times.SSC at about 65.degree. C. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed to identify nucleic acids of this particular embodiment of the invention.

The nucleic acids that make up the subject physical collections may be single-stranded or double-stranded. In addition, the nucleic acids that make up the physical collections may be linear or circular, and the individual nucleic acid molecules may include, in addition to a phenotype determinative gene coding sequence, other sequences, e.g., vector sequences. A variety of different nucleic acids may make up the physical collections, e.g., libraries, such as vector libraries, of the subject invention, where examples of different types of nucleic acids include, but are not limited to, DNA, e.g., cDNA, etc., RNA, e.g., mRNA, cRNA, etc. and the like. The nucleic acids of the physical collections may be present in solution or affixed, i.e., attached to, a solid support, such as a substrate as is found in array embodiments, where further description of such diverse embodiments is provided below. Also provided are virtual collections of the subject phenotype determinative genes. By virtual collection is meant one or more data files or other computer readable data organizational elements that include the sequence information of the genes of the collection, where the sequence information may be the genomic sequence information but is typically the coding sequence information. The virtual collection may be recorded on any convenient computer or processor readable storage medium. The computer or processor readable storage medium on which the collection data is stored may be any convenient medium, including CD, DAT, floppy disk, RAM, ROM, etc, which medium is capable of being read by a hardware component of the device.

Also provided are databases of expression profiles of the phenotype determinative genes. Such databases will typically comprise expression profiles of various cells/tissues having the phenotypes, such as various stages of a disease negative expression profiles, prognostic profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc. As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks expression profiles possessing varying degrees of similarity to a reference expression profile. Such presentation provides a skilled artisan with a ranking of similarities and identifies the degree of similarity contained in the test expression profile.

Specific phenotype determinative genes of the subject invention are those listed in Table 1. Of the list of genes, certain of the genes have functions that logically implicate them as being associated with the phenotype. However, the remaining genes have functions that do not readily associate them with the phenotype.

In certain embodiments, the number of genes in the collection that are from a gene signature of Table 1 is at least 5, at least 10, at least 25, at least 50, at least 75 or more, including all of the genes listed in a gene signature of Table 1 or are preferred Table 1 genes. The subject collections may include only those genes that are listed in Tables 1 or they may include additional genes that are not listed in the tables. Where the subject collections include such additional genes, in certain embodiments the % number of additional genes that are present in the subject collections does not exceed about 50%, usually does not exceed about 25%. In many embodiments where additional “non-Table” genes are included, a great majority of genes in the collection are deregulated pathway determinative genes, where by great majority is meant at least about 75%, usually at least about 80% and sometimes at least about 85, 90, 95% or higher, including embodiments where 100% of the genes in the collection are deregulated pathway determinative genes. In some embodiments, at least one of the genes in the collection is a gene whose function does not readily implicate it in the pathway of interest, where such genes include those genes that are listed in Table 1 but which have not been assigned a biological process. In many embodiments, the subject collections include two or more genes from this group, where the number of genes that are included from this group may be 5, 10, 20 or more, up to and including all of the genes in this group. In some embodiments, the set comprises at least 1, 2, 3, 4, 5, 6, 7, 8, 9, 10, 12, 15, 20, 25, 30, 40 or 50 preferred genes from Table 1. The subject invention provides collections of phenotype determinative genes as determined by the methods of the invention. Although the following disclosure describes subject collections in terms of the genes listed in the Tables relevant to each embodiment of the invention described herein, the subject collections and subsets thereof as claimed by the invention apply to all relevant genes determined by the subject invention. Thus, the subject collections and subsets thereof, as well as applications directed to the use of the aforementioned subject collections only serve as an example to illustrate the invention. The subject collections find use in a number of different applications. Applications of interest include, but are not limited to: (a) diagnostic applications, in which the collections of the genes are employed to either predict the presence of, or the probability for occurrence of, the phenotype; (b) pharmacogenomic applications, in which the collections of genes are employed to determine an appropriate therapeutic treatment regimen, which is then implemented; and (c) therapeutic agent screening applications, where the collection of genes is employed to identify phenotype modulatory agents. Each of these different representative applications is now described in greater detail below.

### Diagnostic Applications

In diagnostic applications of the subject invention, cells or collections thereof, e.g., tissues, as well as animals (subjects, hosts, etc., e.g., mammals, such as pets, livestock, and humans, etc.) that include the cells/tissues are assayed to determine the presence of and/or probability for development of a cancer subtype or the effectiveness of a treatment protocol. As such, diagnostic methods include methods of determining the presence of the phenotype. In certain embodiments, not only the presence but also the severity or stage of a phenotype is determined. In addition, diagnostic methods also include methods of determining the propensity to develop a phenotype, such that a determination is made that the phenotype is not present but is likely to occur.

In practicing the subject diagnostic methods, a nucleic acid sample obtained or derived from a cell, tissue or subject that includes the same that is to be diagnosed is first assayed to generate an expression profile, where the expression profile includes expression data for at least two of the genes listed in each of the tables relevant to the phenotype. The number of different genes whose expression data, i.e., presence or absence of expression, as well as expression level, that are included in the expression profile that is generated may vary, but is typically at least 2, and in many embodiments ranges from 2 to about 100 or more, sometimes from 3 to about 75 or more, including from about 4 to about 70 or more.

As indicated above, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined phenotype exists, including, but not limited, to, breast cancer, ovarian cancer, and/or lung cancer.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Once the expression profile is obtained from the sample being assayed, the expression profile is compared with a reference or control profile to make a diagnosis regarding the phenotype of the cell or tissue from which the sample was obtained/derived. The reference or control profile may be a profile that is obtained from a cell/tissue known to have a phenotype, as well as a particular stage of the phenotype or disease state, and therefore may be a positive reference or control profile. In addition, the reference or control profile may be a profile from cell/tissue for which it is known that the cell/tissue ultimately developed a phenotype, and therefore may be a positive prognostic control or reference profile. In addition, the reference/control profile may be from a normal cell/tissue and therefore be a negative reference/control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has for example, the diseased, or normal phenotype. Furthermore, the obtained expression profile may be compared to a series of positive control/reference profiles each representing a different stage/level of the phenotype (for example, a disease state), so as to obtain more in depth information regarding the particular phenotype of the assayed cell/tissue. The obtained expression profile may be compared to a prognostic control/reference profile, so as to obtain information about the propensity of the cell/tissue to develop the phenotype.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above. The comparison step results in information regarding how similar or dissimilar the obtained expression profile is to the control/reference profiles, which similarity/dissimilarity information is employed to determine the phenotype of the cell/tissue being assayed. For example, similarity with a positive control indicates that the assayed cell/tissue has the phenotype. Likewise, similarity with a negative control indicates that the assayed cell/tissue does not have the phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of a phenotype of an assayed cell/tissue. In addition, where appropriate reference profiles are employed, the above comparison step can yield information about the particular stage of the phenotype of an assayed cell/tissue. Furthermore, the above comparison step can be used to obtain information regarding the propensity of the cell or tissue to develop cancer.

In many embodiments, the above obtained information about the cell/tissue being assayed is employed to diagnose a host, subject or patient with respect to the presence of, state of or propensity to develop, a cancer state. For example, where the cell/tissue that is assayed is determined to have the phenotype, the information may be employed to diagnose a subject from which the cell/tissue was obtained as having the phenotype state, for example, cancer. Exemplary methods of diagnosing deregulated pathways are shown in Example 1-5. The information may also be used to predict the effectiveness of a treatment plan. An exemplary method of predicting a treatment plan is shown in Example 6.

### Reference Profile

In one embodiment of the methods described herein, the reference profile of the methods of this disclosure is the level of gene products in a sample from a normal individual, such as but not limited to, an individual who does not have cancer, or from a non-diseased tissue from a subject afflicted with cancer. If the control sample is from a normal individual, then increased or decreased levels of gene products in the biological sample from the individual being assessed compared to the reference profile indicates that the individual has a deregulated pathway.

The reference profile of gene products can be determined at the same time as the level of gene products in the biological sample from the individual. Alternatively, the reference profile may be a predetermined standard value, or range of values, (e.g. from analysis of other samples) to correlate with deregulation of a pathway. In one specific embodiment, the control value may be data obtained from a data bank corresponding to currently accepted normal levels the gene products under analysis. In situations, such as but not limited to, those where standard data is not available, the methods of the invention may further comprise conducting corresponding analyses in a second set of one or more biological samples from individuals not having cancer, in order to generate the reference profile. Such additional biological samples can be obtained, for example, from unaffected members of the public. An exemplary method of obtaining a reference profile is shown in Example 1.

In the methods of the invention, the comparison of gene product level with the reference profile can be a straight-forward comparison, such as but not limited to, a ratio. The comparison can also involve subjecting the measurement data to any appropriate statistical analysis. In the diagnostic procedures of the invention, one or more biological samples obtained from an individual can be subjected to a battery of analyses in which a desired number of additional genes, gene products, metabolites, and metabolic by-products are measured. In any such diagnostic procedure it is possible that one or more of the measures obtained will produce an inconclusive result. Accordingly, data obtained from a battery of measures can be used to provide for a more conclusive diagnosis and can aid in selection of a normalized reference profile of gene expression. It is for this reason that an interpretation of the data based on an appropriate weighting scheme and/or statistical analysis may be desirable in some embodiments.

### Pharmaco/Surgicogenomic Applications

Another application in which the subject collections of phenotype determinative genes find use in is pharmacogenomic and/or surgicogenomic applications. In these applications, a subject/host/patient is first diagnosed with the deregulated oncogenic pathway, using a protocol such as the diagnostic protocols known to those skilled in the art. The subject is then treated using a pharmacological and/or surgical treatment protocol, where the suitability of the protocol for a particular subject/patient is determined using the results of the diagnosis step. A variety of different pharmacological and surgical treatment protocols are known to those of skill in the art. Such protocols include, but are not limited to: surgical treatment protocols known to those skilled in the art. Pharmacological protocols of interest include treatment with a variety of different types of agents, including but not limited to: thrombolytic agents, growth factors, cytokines, nucleic acids (e.g. gene therapy agents), antineoplastic agents, and chemotherapeutics. An exemplary method of treating samples with the results of a diagnostic step is shown in Example 6.

### Assessment of Therapy (Therametrics)

Another application in which the subject collections of phenotype determinative genes find use is in monitoring or assessing a given treatment protocol. In such methods, a cell/tissue sample of a patient undergoing treatment for a disease condition is monitored using the procedures described above in the diagnostic section, where the obtained expression profile is compared to one or more reference profiles to determine whether a given treatment protocol is having a desired impact on the disease being treated. For example, periodic expression profiles are obtained from a patient during treatment and compared to a series of reference/controls that includes expression profiles of various phenotype (for example, a disease) stages and normal expression profiles. An observed change in the monitored expression profile towards a normal profile indicates that a given treatment protocol is working in a desired manner. In this manner, the degree of deregulation of the pathway may be monitored during treatment.

### Therapeutic Agent Screening Applications

The present invention also encompasses methods for identification of agents having the ability to modulate the activity of a deregulated pathway, e.g., enhance or diminish the phenotype, which finds use in identifying therapeutic agents for a disease. In preferred embodiments, the deregulated pathway is an oncogene or tumor suppressor pathway. Identification of compounds that modulate the activity of a deregulated pathway can be accomplished using any of a variety of drug screening techniques. The screening assays of the invention are generally based upon the ability of the agent to modulate an expression profile of deregulated pathway determinative genes.

The term “agent” as used herein describes any molecule, e.g., protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e., at zero concentration or below the level of detection. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries.

Known pharmacological agents may be subjected to directed or random chemical modifications, such as acylation, alkylation, esterification, amidification, etc. to produce structural analogs.

Exemplary candidate agents of particular interest include, but are not limited to, antisense polynucleotides, and antibodies, soluble receptors, and the like. Antibodies and soluble receptors are of particular interest as candidate agents where the target differentially expressed gene product is secreted or accessible at the cell-surface (e.g., receptors and other molecule stably-associated with the outer cell membrane).

Screening assays can be based upon any of a variety of techniques readily available and known to one of ordinary skill in the art. In general, the screening assays involve contacting a cell or tissue known to have the deregulated pathway with a candidate agent, and assessing the effect upon a gene expression profile made up of deregulated pathway determinative genes. The effect can be detected using any convenient protocol, where in many embodiments the diagnostic protocols described above are employed. Generally such assays are conducted in vitro, but many assays can be adapted for in vivo analyses, e.g., in an animal model of the cancer.

### Screening for Drug Targets

In another embodiment, the invention contemplates identification of genes and gene products from the subject collections of deregulated pathway determinative genes as therapeutic targets. In some respects, this is the converse of the assays described above for identification of agents having activity in modulating (e.g., decreasing or increasing) a phenotype, and is directed towards identifying genes that are deregulated pathway determinative genes as therapeutic targets.

In this embodiment, therapeutic targets are identified by examining the effect(s) of an agent that can be demonstrated or has been demonstrated to modulate a phenotype (e.g., inhibit or suppress a cancer phenotype). For example, the agent can be an antisense oligonucleotide that is specific for a selected gene transcript. For example, the antisense oligonucleotide may have a sequence corresponding to a sequence of a gene appearing in any of the tables relevant to the deregulated pathway determination as taught by the instant invention.

Assays for identification of therapeutic targets can be conducted in a variety of ways using methods that are well known to one of ordinary skill in the art. For example, a test cell that expresses, overexpresses, or underexpresses a candidate gene, e.g., a gene found in Table 1, is contacted with the known agent, the effect upon a cancer phenotype and a biological activity of the candidate gene product assessed. The biological activity of the candidate gene product can be assayed be examining, for example, modulation of expression of a gene encoding the candidate gene product (e.g., as detected by, for example, an increase or decrease in transcript levels or polypeptide levels), or modulation of an enzymatic or other activity of the gene product.

Inhibition or suppression of the cancer phenotype indicates that the candidate gene product is a suitable target for therapy. Assays described herein and/or known in the art can be readily adapted for identification of therapeutic targets. Generally such assays are conducted in vitro, but many assays can be adapted for in vivo analyses, e.g., in an appropriate, art-accepted animal model of the cancer state.

### Reagents and Kits

Also provided are reagents and kits thereof for practicing one or more of the above described methods. The subject reagents and kits thereof may vary greatly. Reagents of interest include reagents specifically designed for use in production of the above described expression profiles of phenotype determinative genes. One type of such reagent is an array probe nucleic acids in which the phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In many embodiments, the arrays include probes for at least 2 of the genes listed in the relevant tables. In certain embodiments, the number of genes that are from the relevant tables that are represented on the array is at least 5, at least 10, at least 25, at least 50, at least 75 or more, including all of the genes listed in the appropriate table. Where the subject arrays include probes for such additional genes, in certain embodiments the number % of additional genes that are represented does not exceed about 50%, usually does not exceed about 25%. In many embodiments a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes. In many embodiments, at least one of the genes represented on the array is a gene whose function does not readily implicate it in the production of the disease phenotype.

Another type of reagent that is specifically tailored for generating expression profiles of phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 2 of the genes listed in Table 1, above. In certain embodiments, the number of genes that are from Table 1 that have primers in the collection is at least 5, at least 10, at least 25, at least 50, at least 75 or more, including all of the genes listed in the relevant table. Where the subject gene specific primer collections include primers for such additional genes, in certain embodiments the number % of additional genes that are represented does not exceed about 50%, usually does not exceed about 25%.

The kits of the subject invention may include the above described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

### Compounds and Methods for Treatment of a Disease Phenotype

Also provided are methods and compositions whereby relevant disease symptoms may be ameliorated. The subject invention provides methods of ameliorating, e.g., treating, disease conditions, by modulating the expression of one or more target genes or the activity of one or more products thereof, where the target genes are one or more of the phenotype determinative genes as determined by the invention.

Certain cancers are brought about, at least in part, by an excessive level of gene product, or by the presence of a gene product exhibiting an abnormal or excessive activity. As such, the reduction in the level and/or activity of such gene products would bring about the amelioration of disease symptoms. Techniques for the reduction of target gene expression levels or target gene product activity levels are discussed below.

Alternatively, certain other diseases are brought about, at least in part, by the absence or reduction of the level of gene expression, or a reduction in the level of a gene product's activity. As such, an increase in the level of gene expression and/or the activity of such gene products would bring about the amelioration of disease symptoms. Techniques for increasing target gene expression levels or target gene product activity levels are discussed below.

Compounds that Inhibit Expression, Synthesis or Activity of Mutant Target Gene Activity

As discussed above, target genes involved in relevant disease disorders can cause such disorders via an increased level of target gene activity. A number of genes are now known to be up-regulated in cells/tissues under disease conditions. A variety of techniques may be utilized to inhibit the expression, synthesis, or activity of such target genes and/or proteins. For example, compounds such as those identified through assays described which exhibit inhibitory activity, may be used in accordance with the invention to ameliorate disease symptoms. As discussed, above, such molecules may include, but are not limited to small organic molecules, peptides, antibodies, and the like. Inhibitory antibody techniques are described, below.

For example, compounds can be administered that compete with an endogenous ligand for the target gene product, where the target gene product binds to an endogenous ligand. The resulting reduction in the amount of ligand-bound gene target will modulate endothelial cell physiology. Compounds that can be particularly useful for this purpose include, for example, soluble proteins or peptides, such as peptides comprising one or more of the extracellular domains, or portions and/or analogs thereof, of the target gene product, including, for example, soluble fusion proteins such as Ig-tailed fusion proteins. (For a discussion of the production of Ig-tailed fusion proteins, see, for example, U.S. Pat. No. 5,116,964.). Alternatively, compounds, such as ligand analogs or antibodies that bind to the target gene product receptor site, but do not activate the protein, (e.g., receptor-ligand antagonists) can be effective in inhibiting target gene product activity. Furthermore, antisense and ribozyme molecules which inhibit expression of the target gene may also be used in accordance with the invention to inhibit the aberrant target gene activity. Such techniques are described, below. Still further, also as described, below, triple helix molecules may be utilized in inhibiting the aberrant target gene activity.

### Inhibitory Antisense, Ribozyme and Triple Helix Approaches

Among the compounds which may exhibit the ability to ameliorate disease symptoms are antisense, ribozyme, and triple helix molecules. Such molecules may be designed to reduce or inhibit mutant target gene activity. Techniques for the production and use of such molecules are well known to those of skill in the art. Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding target gene proteins. Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays. Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex. It is possible that the antisense, ribozyme, and/or triple helix molecules described herein may reduce or inhibit the transcription (triple helix) and/or translation (antisense, ribozyme) of mRNA produced by both normal and mutant target gene alleles. In order to ensure that substantially normal levels of target gene activity are maintained, nucleic acid molecules that encode and express target gene polypeptides exhibiting normal activity may be introduced into cells via gene therapy methods such as those described, below, that do not contain sequences susceptible to whatever antisense, ribozyme, or triple helix treatments are being utilized. Alternatively, it may be preferable to co-administer normal target gene protein into the cell or tissue in order to maintain the requisite level of cellular or tissue target gene activity.

Anti-sense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

### Antibodies for Target Gene Products

Antibodies that are both specific for target gene protein and interfere with its activity may be used to inhibit target gene function. Such antibodies may be generated using standard techniques known in the art against the proteins themselves or against peptides corresponding to portions of the proteins. Such antibodies include but are not limited to polyclonal, monoclonal, Fab fragments, single chain antibodies, chimeric antibodies, etc. In instances where the target gene protein is intracellular and whole antibodies are used, internalizing antibodies may be preferred. However, lipofectin liposomes may be used to deliver the antibody or a fragment of the Fab region which binds to the target gene epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (e.g., see Creighton, 1983, supra; and Sambrook et al., 1989, supra). Alternatively, single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (Marasco, W. et al., 1993, Proc. Natl. Acad. Sci. USA 90:7889-7893).

In some instances, the target gene protein is extracellular, or is a transmembrane protein. Antibodies that are specific for one or more extracellular domains of the gene product, for example, and that interfere with its activity, are particularly useful in treating disease. Such antibodies are especially efficient because they can access the target domains directly from the bloodstream. Any of the administration techniques described, below which are appropriate for peptide administration may be utilized to effectively administer inhibitory target gene antibodies to their site of action.

### Methods for Restoring Target Gene Activity

Target genes that cause the relevant disease may be underexpressed within known disease situations. Several genes are now known to be down-regulated under disease conditions. Alternatively, the activity of target gene products may be diminished, leading to the development of disease symptoms. Described in this section are methods whereby the level of target gene activity may be increased to levels wherein disease symptoms are ameliorated. The level of gene activity may be increased, for example, by either increasing the level of target gene product present or by increasing the level of active target gene product which is present.

For example, a target gene protein, at a level sufficient to ameliorate disease symptoms may be administered to a patient exhibiting such symptoms. Any of the techniques discussed, below, may be utilized for such administration. One of skill in the art will readily know how to determine the concentration of effective, non-toxic doses of the normal target gene protein, utilizing techniques known to those of ordinary skill in the art.

Additionally, RNA sequences encoding target gene protein may be directly administered to a patient exhibiting disease symptoms, at a concentration sufficient to produce a level of target gene protein such that disease symptoms are ameliorated. Any of the techniques discussed, below, which achieve intracellular administration of compounds, such as, for example, liposome administration, may be utilized for the administration of such RNA molecules. The RNA molecules may be produced, for example, by recombinant techniques as is known in the art.

Further, patients may be treated by gene replacement therapy. One or more copies of a normal target gene, or a portion of the gene that directs the production of a normal target gene protein with target gene function, may be inserted into cells using vectors which include, but are not limited to adenovirus, adeno-associated virus, and retrovirus vectors, in addition to other particles that introduce DNA into cells, such as liposomes. Additionally, techniques such as those described above may be utilized for the introduction of normal target gene sequences into human cells. Cells, preferably, autologous cells, containing normal target gene expressing gene sequences may then be introduced or reintroduced into the patient at positions which allow for the amelioration of disease symptoms. Such cell replacement techniques may be preferred, for example, when the target gene product is a secreted, extracellular gene product.

### Pharmaceutical Preparations and Methods of Administration

The identified compounds that inhibit target gene expression, synthesis and/or activity can be administered to a patient at therapeutically effective doses to treat or ameliorate the relevant disease. A therapeutically effective dose refers to that amount of the compound sufficient to result in amelioration of symptoms of disease. Toxicity and therapeutic efficacy of such compounds can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED.sub.50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC.sub.50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### Therapeutic Agents

In certain embodiments, the therapeutic agents of the disclosure may include antineoplastic agents. Antineoplastic agents include, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alpha, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine.

In one embodiment, the antineoplastic agent is 5-Fluoruracil, 6-mercaptopurine, Actinomycin, Adriamycin®, Adrucil®, Aminoglutethimide, Anastrozole, Aredia®, Arimidex®, Aromasin®, Bonefos®, Bleomycin, carboplatin, Cactinomycin, Capecitabine, Cisplatin, Clodronate, Cyclophosphamide, Cytadren®, Cytoxan®, Dactinomycin, Docetaxel, Doxyl®, Doxorubicin, Epirubicin, Etoposide, Exemestane, Femara®, Fluorouracil, Fluoxymesterone, Halotestin®, Herceptin®, Letrozole, Leucovorin calcium, Megace®, Megestrol acetate, Methotrexate, Mitomycin, Mitoxantrone, Mutamycin®, Navelbine®, Nolvadex®, Novantrone®, Oncovin®, Ostac®, Paclitaxel, Pamidronate, Pharmorubicin®, Platinol®, prednisone, Procytox®, Tamofen®, Tamone®, Tamoplex®, Tamoxifen, Taxol®, Taxotere®, Trastuzumab, Thiotepa, Velbe®, Vepesid®, Vinblastine, Vincristine, Vinorelbine, Xeloda®, or a combination thereof.

In another embodiment, the antineoplastic agent comprises a monoclonal antibody, a humanized antibody, a chimeric antibody, a single chain antibody, or a fragment of an antibody. Exemplary antibodies include, but are not limited to, Rituxan, IDEC-C2B8, anti-CD20 Mab, Panorex, 3622W94, anti-EGP40 (17-1A) pancarcinoma antigen on adenocarcinomas Herceptin, Erbitux, anti-Her2, Anti-EGFr, BEC2, anti-idiotypic-GD₃ epitope, Ovarex, B43.13, anti-idiotypic CA125, 4B5, Anti-VEGF, RhuMAb, MDX-210, anti-HER2, MDX-22, MDX-220, MDX-447, MDX-260, anti-GD-2, Quadramet, CYT-424, IDEC-Y2B8, Oncolym, Lym-1, SMART M195, ATRAGEN, LDP-03, anti-CAMPATH, ior t6, anti CD6, MDX-11, OV103, Zenapax, Anti-Tac, anti-IL-2 receptor, MELIMMUNE-2, MELIMMUNE-1, CEACIDE, Pretarget, NovoMAb-G2, TNT, anti-histone, Gliomab-H, GNI-250, EMD-72000, LymphoCide, CMA 676, Monopharm-C, anti-FLK-2, SMART 1D10, SMART ABL 364, ImmuRAIT-CEA, or combinations thereof.

In yet another embodiment, the antineoplastic agent comprises an additional type of tumor cell. In a specific embodiment, the additional type of tumor cell is a MCF-10A, MCF-10F, MCF-10-2A, MCF-12A, MCF-12F, ZR-75-1, ZR-75-30, UACC-812, UACC-893, HCC38, HCC70, HCC202, HCC1007 BL, HCC1008, HCC1143, HCC1187, HCC1187 BL, HCC1395, HCC1569, HCC1599, HCC1599 BL, HCC1806, HCC1937, HCC1937 BL, HCC1954, HCC1954 BL, HCC2157, Hs 274.T, Hs 281.T, Hs 343.T, Hs 362.T, Hs 574.T, Hs 579.Mg, Hs 605.T, Hs 742.T, Hs 748.T, Hs 875.T, MB 157, SW527, 184A1, 184B5, MDA-MB-330, MDA-MB-415, MDA-MB-435S, MDA-MB-436, MDA-MB-453, MDA-MB-468 RT4, BT-474, CAMA-1, MCF7 [MCF-7], MDA-MB-134-VI, MDA-MB-157, MDA-MB-175-VII HTB-27 MDA-MB-361, SK-BR-3 or ME-180 cell, all of which are available from ATTC.

In another embodiment, the antineoplastic agent comprises a tumor antigen. In one specific embodiment, the tumor antigen is her2/neu. Tumor antigens are well-known in the art and are described in U.S. Pat. Nos. 4,383,985 and 5,665,874, in U.S. Patent Publication No. 2003/0027776, and International PCT Publications Nos. WO00/55173, WO00/55174, WO00/55320, WO00/55350 and WO00/55351.

In another embodiment, the antineoplastic agent comprises an antisense reagent, such as an siRNA or a hairpin RNA molecule, which reduces the expression or function of a gene that is expressed in a cancer cell. Exemplary antisense reagents which may be used include those directed to mucin, Ha-ras, VEGFR1 or BRCA1. Such reagents are described in U.S. Pat. Nos. 6,716,627 (mucin), 6,723,706 (Ha-ras), 6,710,174 (VEGFR1) and in U.S. Patent Publication No. 2004/0014051 (BRCA1).

In another embodiment, the antineoplastic agent comprises cells autologous to the subject, such as cells of the immune system such as macrophages, T cells or dendrites. In some embodiments, the cells have been treated with an antigen, such as a peptide or a cancer antigen, or have been incubated with tumor cells from the patient. In one embodiment, autologous peripheral blood lymphocytes may be mixed with SV-BR-1 cells and administered to the subject. Such lymphocytes may be isolated by leukaphoresis. Suitable autologous cells which may be used, methods for their isolation, methods of modifying said cells to improve their effectiveness and formulations comprising said cells are described in U.S. Pat. Nos. 6,277,368, 6,451,316, 5,843,435, 5,928,639, 6,368,593 and 6,207,147, and in International PCT Publications Nos. WO04/021995 and WO00/57705.

In a preferred embodiment, the therapeutic agents of this disclosure may be inhibitors of hyperactivated pathways or activators of hypoactivated pathways in tumours. The therapeutic agents may target oncogenic pathways. In certain embodiments, the therapeutic agent targets one or more members of a pathway. The therapeutic agents of the disclosure include, but are not limited to, chemical compounds, drugs, peptides, antibodies or derivative thereof and RNAi reagents. In the most preferred embodiments, the therapeutic agents may target the Ras, Myc, β-catenin, E2F3 or Src pathways. In some embodiments, inhibitors of the Ras pathway may be farnesyl transferase inhibitors or farnesylthiosalicylic acid. In some embodiments, inhibitors of the Myc pathway may be 10058-F4 (see Yin, X., et al. 2003. Oncogene 22, 6151). In some embodiments, the Src inhibitor may be SU6656 or PP2 (see Boyd et al., Clinical Cancer Research Vol. 10, 1545-1555, February 2004). In certain embodiments, the therapeutic agent of the disclosure may be all or a combination of these agents.

In some embodiments of the methods described herein directed to the treatment of cancer, the subject is treated prior to, concurrently with, or subsequently to the treatment with the cells of the present invention, with a complementary therapy to the cancer, such as surgery, chemotherapy, radiation therapy, or hormonal therapy or a combination thereof.

In a specific embodiment where the cancer is breast cancer, the complementary treatment may comprise breast-sparing surgery i.e. an operation to remove the cancer but not the breast, also called breast-sparing surgery, breast-conserving surgery, lumpectomy, segmental mastectomy, or partial mastectomy. In another embodiment, it comprises a mastectomy. A mastectomy is an operation to remove the breast, or as much of the breast tissue as possible, and in some cases also the lymph nodes under the arm. In yet another embodiment, the surgery comprises sentinel lymph node biopsy, where only one or a few lymph nodes (the sentinel nodes) are removed instead of removing a much larger number of underarm lymph nodes. Surgery may also comprise modified radical mastectomy, where a surgeon removes the whole breast, most or all of the lymph nodes under the arm, and, often, the lining over the chest muscles. The smaller of the two chest muscles also may be taken out to make it easier to remove the lymph nodes.

In a specific embodiment where the cancer is ovarian cancer, the complementary treatment may comprise surgery in addition to another form of treatment (e.g., chemotherapy and/or radiotherapy). Surgery may comprise a total hysterectomy (removal of the uterus [womb]), bilateral salpingo-oophorectomy (removal of the fallopian tubes and ovaries on both sides), omentectomy (removal of the fatty tissue that covers the bowels), and lymphadenectomy (removal of one or more lymph nodes).

In a specific embodiment where the cancer is NSCLC, the complementary treatment may comprise adjuvant cisplatin-based combination chemotherapy or radiation therapy in combination with chemotherapy depending on the stage of the tumor (see Albain et al., J Clin Oncol 9 (9): 1618-26, 1991).

In a specific embodiment, the complementary treatment comprises radiation therapy. Radiation therapy may comprise external radiation, where radiation comes from a machine, or from internal radiation (implant radiation, wherein the radiation originates from radioactive material placed in thin plastic tubes put directly in the breast.

In another specific embodiment, the complementary treatment comprises chemotherapy. Chemotherapeutic agents found to be of assistance in the suppression of tumors include but are not limited to alkylating agents (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkaloids and antibiotics). In a specific embodiment, the chemotherapeutic agent is selected from the group consisting of allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorubicin citrate, idarubicin HCL, plimycin, mitomycin, pentostatin, mitoxantrone, valrubicin, cytarabine, fludarabine phosphate, floxuridine, cladribine, methotrexate, mercaptopurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alfa-2b, mitotane, procarbazine HCL, vinorelbine tartrate, E. coli L-asparaginase, Erwinia L-asparaginase, vincristine sulfate, denileukin diftitox, aldesleukin, rituximab, interferon alfa-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, and diamino dichloro platinum, said chemotherapy agent in combination with thymosinα₁ being administered in an amount effective to reduce said side effects of chemotherapy in said patient.

In another specific embodiment, the complementary treatment comprises hormonal therapy. Hormonal therapy may comprise the use of a drug, such as tamoxifen, that can block the natural hormones like estrogen or may comprise aromatase inhibitors which prevent the synthesis of estradiol. Alternative, hormonal therapy may comprise the removal of the subject's ovaries, especially if the subject is a woman who has not yet gone through menopause.

### Methods of Identifying Deregulated Pathway Determinative Genes

Also provided are methods of identifying deregulated pathway determinative genes, i.e., genes whose expression is associated with a disease phenotype (see US Patent Application No. 20050170528 and 20030224383).

In these methods, an expression profile for a nucleic acid sample obtained from a source having the deregulated pathway phenotype, or from a diseased tissue suspected of having a deregulated pathway, is prepared using the gene expression profile generation techniques described above, with the only difference being that the genes that are assayed are candidate genes and not genes necessarily known to be deregulated pathway determinative genes. Next, the obtained expression profile is compared to a control profile, e.g., obtained from a source that does not have a deregulated pathway phenotype. Following this comparison step, genes whose expression correlates with said the deregulated pathway are identified. In certain embodiments, the correlation is based on at least one parameter that is other than expression level. As such, a parameter other than whether a gene is up or down regulated is employed to find a correlation of the gene with the deregulated pathway phenotype.

One expression analysis approach may include a Bayesian analysis of binary prediction tree models for retrospectively sampled outcomes as illustrated in the following three exemplary analyses.

Bayesian analysis is an approach to statistical analysis that is based on the Bayes law, which states that the posterior probability of a parameter p is proportional to the prior probability of parameter p multiplied by the likelihood of p derived from the data collected. This increasingly popular methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon. Bayesian analysis have been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” modes using a splitting rule. For instance, for clarification, training data contains sample vectors that have one or more measurement variables and one variable that determines that class of the sample. Various splitting rules have been used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. A statistical predictive tree model to which Bayesian analysis is applied may consistently deliver accurate results with high predictive capabilities.

Development of the Tree Clarification Model: Model Context and Methodology Data {Zi, xᵢ} (i=1, . . . , n) are available on a binary response variable Z and a p-dimensional covariate vector x: The 0/1 response totals are fixed by design. Each predictor variable xⱼ could be binary, discrete or continuous.

### 1. Bayes' Factor Measures of Association

At the heart of a classification tree is the assessment of association between each predictor and the response in subsamples, and we first consider this at a general level in the full sample. For any chosen single predictor x; a specified threshold_on the levels of x organizes the data into the 2×2 table.

| | Z = 0 | Z = 1 | |
|---|---|---|---|
| x ≦ T | n₀₀ | n₀₁ | N₀ |
| x > T | n₁₀ | n₁₁ | N₁ |
| | M₀ | M₁ | |

With column totals fixed by design, the categorized data is properly viewed as two Bernoulli sequences within the two columns, hence sampling

p(n_{0z},n_{1z}|M_{z},θ_{z,τ})=θ_{z,τ}ⁿ^{_{0z}}(1−θ_{z,τ})ⁿ^{_{1z}}

for each column z=0, 1. Here, of course, θ_{0,τ}=Pτ(x≦τ|Z=0) and θ_{1,τ}=Pτ(x≦τ|z=1). A test of association of the thresholded predictor with the response will now be based on assessing the difference between these Bernoulli probabilities.

The natural Bayesian approach is via the Bayes' factor B_{τ} comparing the null hypothesis θ_{0,τ}=θ_{1,τ} to the full alternative θ_{0,τ}≠θ_{1,τ}. We adopt the standard conjugate beta prior model and require that the null hypothesis be nested within the alternative. Thus, assuming θ_{0,τ}≠θ_{1,τ}, we take θ_{0,τ} and θ_{1,τ} to be independent with common prior Be(a_{τ}, b_{τ}) with mean m_{τ}=a_{τ}/(a_{τ}+b_{τ}). On the null hypothesis θ_{0,τ}=θ_{1,τ}, the common value has the same beta prior. The resulting Bayes' factor in favour of the alternative over the null hypothesis is then simply

$${B}_{τ} = \frac{β ( {n}_{00} + {a}_{τ} , {n}_{10} + {b}_{τ} ) β ( {n}_{01} + {a}_{τ} , {n}_{11} + {b}_{τ} )}{β ( {N}_{0} + {a}_{τ} , {N}_{1} + {b}_{τ} ) β ( {a}_{τ} , {b}_{τ} )} .$$

As a Bayes' factor, this is calibrated to a likelihood ratio scale. In contrast to more traditional significance tests and also likelihood ratio approaches, the Bayes' factor will tend to provide more conservative assessments of significance, consistent with the general conservative properties of proper Bayesian tests of null hypotheses (See Sellke, T., Bayarri, M. J. and Berger, J. O., Calibration of p_values for testing precise null hypotheses, The American Statistician, 55, 62-71, (2001) and references therein).
In the context of comparing predictors, the Bayes' factor Bτ may be evaluated for all predictors and, for each predictor, for any specified range of thresholds. As the threshold varies for a given predictor taking a range of (discrete or continuous) values, the Bayes' factor maps out a function of τ and high values identify ranges of interest for thresholding that predictor. For a binary predictor, of course, the only relevant threshold to consider is τ=0.
2. Model Consistency with Respect to Varying Thresholds

A key question arises as to the consistency of this analysis as we vary the thresholds. By construction, each probability θ_{Zτ} is a non-decreasing function of τ, a constraint that must be formally represented in the model. The key point is that the beta prior specification must formally reflect this. To see how this is achieved, note first that θ_{Zτ} is in fact the cumulative distribution function of the predictor values χ; conditional on Z=z; (z=0; 1); evaluated at the point χ=τ. Hence the sequence of beta priors, Be(a_{τ}, b_{τ}) as τ varies, represents a set of marginal prior distributions for the corresponding set of values of the cdfs. It is immediate that the natural embedding is in a non-parametric Dirichlet process model for the complete cdf. Thus the threshold-specific beta priors are consistent, and the resulting sets of Bayes' factors comparable as τ varies, under a Dirichlet process prior with the betas as margins. The required constraint is that the prior mean values m_{τ} are themselves values of a cumulative distribution function on the range of χ, one that defines the prior mean of each θ_{τ} as a function. Thus, we simply rewrite the beta parameters (α_{τ}, b_{τ}) as α_{τ}=αm_{τ} and b_{τ}=α(1−m_{τ}) for a specified prior mean cdf m_{τ}, and where α is the prior precision (or “total mass”) of the underlying Dirichlet process model. Note that this specializes to a Dirichlet distribution when χ is discrete on a finite set of values, including special cases of ordered categories (such as arise if χ is truncated to a predefined set of bins), and also the extreme case of binary χ when the Dirichlet is a simple beta distribution.

### 3. Generating a Tree

The above development leads to a formal Bayes' factor measure of association that may be used in the generation of trees in a forward-selection process as implemented in traditional classification tree approaches. Consider a single tree and the data in a node that is a candidate for a binary split. Given the data in this node, construct a binary split based on a chosen (predictor, threshold) pair (χ, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 3 prior, Bayes' factors of 2.2, 2.9, 3.7 and 5.3 correspond, approximately, to probabilities of 0.9, 0.95, 0.99 and 0.995, respectively. This guides the choice of threshold, which may be specified as a single value for each level of the tree. We have utilized Bayes' factor thresholds of around 3 in a range of analyses, as exemplified below. Higher thresholds limit the growth of trees by ensuring a more stringent test for splits.

The Bayes' factor measure will always generate less extreme values than corresponding generalized likelihood ratio tests (for example), and this can be especially marked when the sample sizes M₀ and M₁ are low. Thus the propensity to split nodes is always generally lower than with traditional testing methods, especially with lower samples sizes, and hence the approach tends to be more conservative in extending existing trees. Post-generation pruning is therefore generally much less of an issue, and can in fact generally be ignored.

Index the root node of any tree by zero, and consider the full data set of n observations, representing M_{z} outcomes with Z=z in 0, 1. Label successive nodes sequentially: splitting the root node, the left branch terminates at node 1, the right branch at node 2; splitting node 1, the consequent left branch terminates at node 3, the right branch at node 4; splitting node 2, the consequent left branch terminates at node 5, and the right branch at node 6, and so forth. Any node in the tree is labelled numerically according to its “parent” node; that is, a node j splits into two children, namely the (left, right) children (2j+1; 2j+2): At level m of the tree (n=0; 1; : : : ;) the candidates nodes are, from left to right, as 2^{m}_{—}1; 2^{m}; : : : ; 2^{m+1}−2.

Having generated a “current” tree, we run through each of the existing terminal nodes one at a time, and assess whether or not to create a further split at that node, stopping based on the above Bayes' factor criterion. Unless samples are very large (thousands) typical trees will rarely extend to more than three or four levels.

4. Inference and Prediction with a Single Tree

Suppose we have generated a tree with m levels; the tree has some number of terminal nodes up to the maximum possible of L=2^{m+1}−2. Inference and prediction involves computations for branch probabilities and the predictive probabilities for new cases that these underlie. We detail this for a specific path down the tree, i.e., a sequence of nodes from the root node to a specified terminal node.

First, consider a node j that is split based on a (predictor, threshold) pair labeled (χⱼ, τⱼ), (note that we use the node index to label the chosen predictor, for clarity). Extend the notation of Section 2.1 to include the subscript j indexing this node. Then the data at this node involves M₀ⱼ cases with Z=0 and M₁ⱼ cases with Z=1. Based on the chosen (predictor, threshold) pair (χⱼ, τⱼ) these samples split into cases n₀₀ⱼ, n₀₁ⱼ, n₁₀ⱼ, n₁₁ⱼ as in the table of Section 2.1, but now indexed by the node label j. The implied conditional probabilities θ_{z,τ,j}=Pr(χⱼ≦τⱼ|Z=z), for z=0, 1 are the branch probabilities defined by such a split (note that these are also conditional on the tree and data subsample in this node, though the notation does not explicitly reflect this for clarity). These are uncertain parameters and, following the development of Section 2.1, have specified beta priors, now also indexed by parent node j, i.e., Be(a_{τ,j}, b_{τ,j}). Assuming the node is split, the two sample Bernoulli setup implies conditional posterior distributions for these branch probability parameters: they are independent with posterior beta distributions

θ_{0,τ,j}˜Be(a_{τj}+n₀₀ⱼ,b_{τj}+n₁₀ⱼ) and θ_{1,τj˜Be(a}_{τ,j}+n₀₁ⱼ,b_{τ,j}+n₁₁ⱼ).

These distributions allow inference on branch probabilities, and feed into the predictive inference computations as follows.

Consider predicting the response Z* of a new case based on the observed set of predictor values x*. The specified tree defines a unique path from the root to the terminal node for this new case. To predict requires that we compute the posterior predictive probability for Z*=1/0. We do this by following x* down the tree to the implied terminal node, and sequentially building up the relevant likelihood ratio defined by successive (predictor, threshold) pairs.

For example and specificity, suppose that the predictor profile of this new case is such that the implied path traverses nodes 0, 1, 4, 9, terminating at node 9. This path is based on a (predictor, threshold) pair (χ₀, τ₀) that defines the split of the root node, (χ₁, τ₁) that defines the split of node 1, and (χ₄, τ₄) that defines the split of node 4. The new case follows this path as a result of its predictor values, in sequence: (x*₀≦τ₀), (x*₁>τ₁) and (x*₄≦τ₄). The implied likelihood ratio for Z*=1 relative to Z*=0 is then the product of the ratio of branch probabilities to this terminal node, namely

$${λ}^{*} = \frac{{θ}_{1 , {τ}_{0} , 0}}{{θ}_{0 , {τ}_{0} , 0}} \times \frac{( 1 - {θ}_{1 , {τ}_{1} , 1} )}{( 1 - {θ}_{0 , {τ}_{1} , 1} )} \times \frac{{θ}_{1 , {τ}_{0} , 0}}{{θ}_{0 , {τ}_{0} , 0}} .$$

Hence, for any specified prior probability Pr(Z*=1), this single tree model implies that, as a function of the branch probabilities, the updated probability π* is, on the odds scale, given by

$$\frac{{π}^{*}}{( 1 - {π}^{*} )} = {λ}^{*} \frac{P r ( {Z}^{*} = 1 )}{P r ( {Z}^{*} = 0 )} .$$

Hence, for any specified prior probability π Pr(Z*=1), this single tree model implies that, as a function the branch probabilities, the updated probability π* is, on the odds scale, given by

$$\frac{{π}^{*}}{( 1 - {π}^{*} )} = {λ}^{*} \frac{P r ( {Z}^{*} = 1 )}{P r ( {Z}^{*} = 0 )}$$

The case-control design provides no information about Pr(Z*=1) so it is up to the user to specify this or examine a range of values; one useful summary is obtained by simply taking a 50:50 prior odds as benchmark, whereupon the posterior probability is π*=λ*/(1+λ*).
Prediction follows by estimating π* based on the sequence of conditionally independent posterior distributions for the branch probabilities that define it. For example, simply “plugging-in” the conditional posterior means of each θ. will lead to a plug-in estimate of λ* and hence π*. The full posterior for π* is defined implicitly as it is a function of the θ. Since the branch probabilities follow beta posteriors, it is trivial to draw Monte Carlo samples of the θ. and then simply compute the corresponding values of λ* and hence π* to generate a posterior sample for summarization. This way, we can evaluate simulation-based posterior means and uncertainty intervals for π* that represent predictions of the binary outcome for the new case.

### 5. Generating and Weighting Multiple Trees

In considering potential (predictor, threshold) candidates at any node, there may be a number with high Bayes' factors, so that multiple possible trees with difference splits at this node are suggested. With continuous predictor variables, small variations in an “interesting” threshold will generally lead to small changes in the Bayes' factor—moving the threshold so that a single observation moves from one side of the threshold to the other, for example. This relates naturally to the need to consider thresholds as parameters to be inferred; for a given predictor χ, multiple candidate splits with various different threshold values τ reflects the inherent uncertainty about τ, and indicates the need to generate multiple trees to adequately represent that uncertainty. Hence, in such a situation, the tree generation can spawn multiple copies of the “current” tree, and then each will split the current node based on a different threshold for this predictor. Similarly, multiple trees may be spawned this way with the modification that they may involve different predictors. In problems with many predictors, this naturally leads to the generation of many trees, often with small changes from one to the next, and the consequent need for careful development of tree-managing software to represent the multiple trees. In addition, there is then a need to develop inference and prediction in the context of multiple trees generated this way. The use of “forests of trees” has recently been urged by Breiman, L., Statistical Modeling: The two cultures (with discussion), Statistical Science, 16 199-225 (2001), and our perspective endorses this. The rationale here is quite simple: node splits are based on specific choices of what we regard as parameters of the overall predictive tree model, the (predictor, threshold) pairs. Inference based on any single tree chooses specific values for these parameters, whereas statistical learning about relevant trees requires that we explore aspects of the posterior distribution for the parameters (together with the resulting branch probabilities). Within the current framework, the forward generation process allows easily for the computation of the resulting relative likelihood values for trees, and hence to relevant weighting of trees in prediction. For a given tree, identify the subset of nodes that are split to create branches. The overall marginal likelihood function for the tree is then the product of component marginal likelihoods, one component from each of these split nodes. Continue with the notation of Section 2.1 but now, again, indexed by any chosen node j: Conditional on splitting the node at the defined (predictor, threshold) pair (χⱼ, τⱼ), the marginal likelihood component is

$${m}_{j} = {\int }_{0}^{1} {\int }_{0}^{1} {\prod }_{z = 0 , 1} p ( {n}_{0 zj} , {n}_{1 zj} \vert {M}_{zj} , {θ}_{z , {τ}_{j} , j} ) p ( {θ}_{z , {τ}_{j} , j} ) d {θ}_{z , {τ}_{j} , j}$$

where p(θ_{z,τ,j},j) is the Be(a_{τ,j}, b_{τ,j}) prior for each z=0, 1. This clearly reduces to

$${m}_{j} = {\prod }_{z = 0 , 1} \frac{β ( {n}_{0 zj} + {a}_{τ , j} , {n}_{1 zj} + {b}_{τ , j} )}{β ( {a}_{τ , j} , {b}_{τ , j} )} .$$

The overall marginal likelihood value is the product of these terms over all nodes j that define branches in the tree. This provides the relative likelihood values for all trees within the set of trees generated. As a first reference analysis, we may simply normalize these values to provide relative posterior probabilities over trees based on an assumed uniform prior. This provides a reference weighting that can be used to both assess trees and as posterior probabilities with which to weight and average predictions for future cases.

### EXAMPLE 1

### Development Of Pathway Signatures

Human primary mammary epithelial cell cultures (HMEC) were used to develop a series of pathway signatures. Recombinant adenoviruses were employed to express various oncogenic activities in an otherwise quiescent cell, thereby specifically isolating the subsequent events as defined by the activation/deregulation of that single pathway. Various biochemical measures demonstrate pathway activation (FIG. 5). RNA from multiple independent infections was collected for DNA microarray analysis using Affymetrix Human Genome U133 Plus 2.0 Array. Gene expression signatures that reflect the activity of a given pathway are identified using supervised classification methods of analysis previously described¹² The analysis selects a set of genes whose expression levels are most highly correlated with the classification of cell line samples into oncogene-activated/deregulated versus control (GFP). The dominant principal components from such a set of genes then defines a relevant phenotype-related metagene, and regression models assign the relative probability of pathway deregulation in tumor or cell line samples.

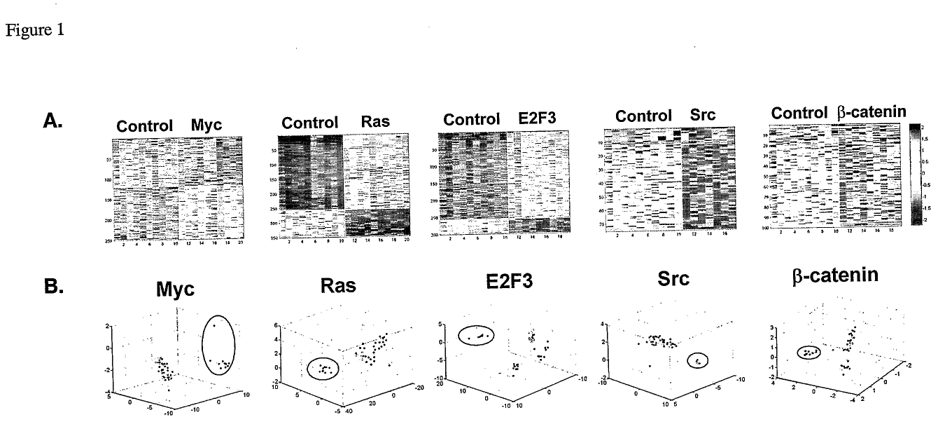

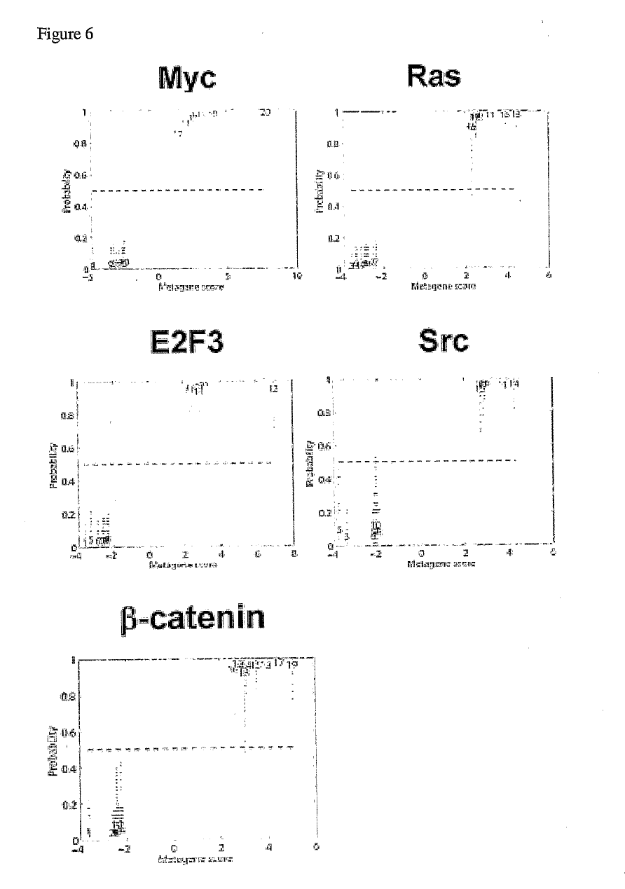

It is clear from FIG. 1A that the various signatures distinguish cells expressing the oncogenic activity from control cells. Given the potential for overlap in the pathways, the extent to which the signatures distinguish one pathway from another was examined. Use of the first three principal components from each signature, evaluated across all experimental samples, demonstrates that the patterns of expression in each signature are specific to each pathway; the gene expression patterns accurately distinguish the individual oncogenic effects despite overlapping downstream consequences (FIG. 1B). The genes identified as comprising each signature are listed in Table 1. To more formally evaluate the predictive validity and robustness of the pathway signatures, a leave-one-out cross validation study was applied to the set of pathway predictors. This analysis demonstrates that these signatures of oncogenic pathways can accurately predict the cells expressing the oncogenic activity from the control cells (FIG. 6). The analysis clearly distinguishes and predicts the state of an oncogenic pathway.

### EXAMPLE 2

### Detection of Deregulated Pathways in Mouse Cancer Models

Further verification of the capacity of oncogenic pathway signatures to accurately predict the status of pathways made use of tumor samples derived from various mouse cancer models. Pathway signatures were regenerated from the genes common to both human and mouse data sets; the analysis was trained on the cell line data and then used to predict the pathway status of all tumors. These studies were carried out using three of the pathway signatures for which matching mouse models were available that could be used for validation: Myc, Ras, and E2F3. Across the set of mouse tumors, this analysis evaluates the relative probability of pathway deregulation of each tumor—that is, the predicted status of the pathway in each mouse tumor based only on the signatures developed in cell lines.

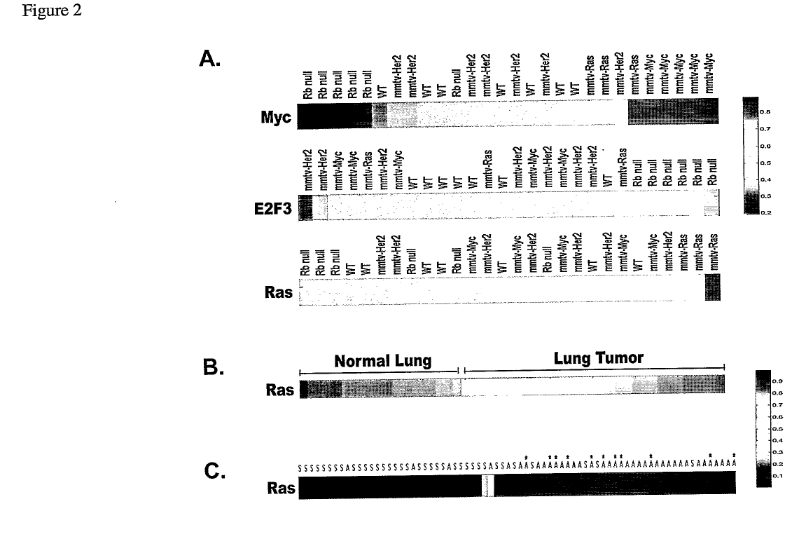

These predictions are displayed as a color map: high probability of pathway deregulation (red) and low probability (blue), with predictions sorted by the relative probability of pathway deregulation. As shown in FIG. 2A, the pathway predictions exhibit close correlation with the molecular basis for the tumor induction. For instance, the five MMTV-Myc tumors exhibit the highest probability of Myc pathway deregulation, while the six Rb null tumors exhibit the highest probability of E2F3 deregulation. The probability of Ras pathway activation was highest in the MMTV-Ras animals and MMTV-Myc tumors; this indication of Ras pathway activation in the MMTV-Myc tumors is consistent with past results demonstrating a selection for Ras mutations in these tumors^{6,13}.

Further substantiation and validation was obtained from a series of tumors in which Ras activity was spontaneously activated by homologous recombination in adult animals, more closely mimicking pathway deregulation in human tumors¹⁴. There was a consistent prediction of Ras pathway deregulation within these tumors when compared to the set of samples from control lung tissue (FIG. 2B). Taken together, these results strongly support the conclusion that the various oncogenic pathway signatures do reliably reflect pathway status under a variety of circumstances and thus can serve as useful tools to probe the status of these pathways.

### EXAMPLE 3

### Detection of Deregulated Pathways in Lung Cancer

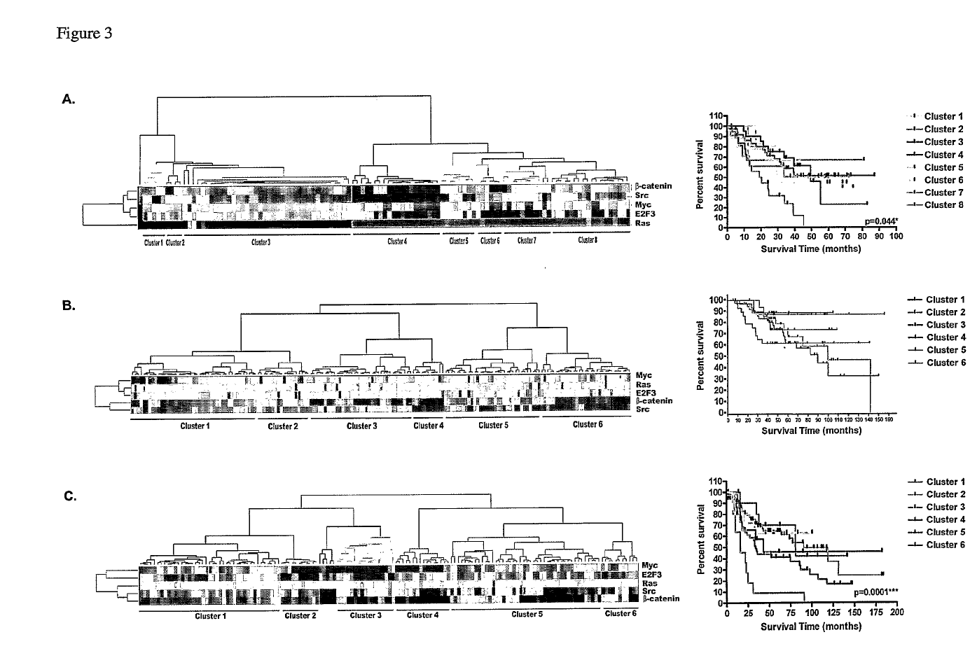

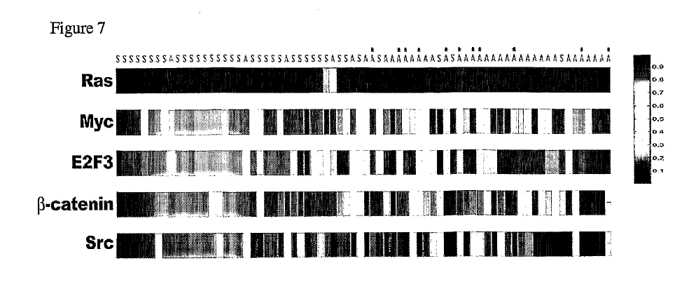

Previous work has linked Ras activation with development of adenocarcinomas of the lung^{15,16}. A set of non-small cell lung carcinoma samples were used to predict the pathway status and then sorted according to predicted Ras activity. As shown in FIG. 2C, Ras pathway status very clearly correlates with the histological subtype—the majority of the adenocarcinoma samples (‘A’) exhibit a high probability of Ras deregulation relative to the squamous cell carcinoma samples (‘S’). Prediction of the status of the other pathways revealed a less distinct pattern although each tended to be more active in the squamous cell carcinoma samples (FIG. 7). This pattern becomes more evident in the analysis shown in FIG. 3. An examination of Ras mutation identified 11 samples with K-Ras mutations, all confined to the adenocarcinomas (indicated by * in the figure) (Table 2). Overall, 14% of NSCLC tumors and 29% of the adenocarcinomas had K-Ras mutations in codon 12. Since nearly all of the adenocarcinomas exhibited Ras pathway deregulation, it appears that deregulation of Ras pathway is indeed a characteristic of development of adenocarcinoma of the lung and that this can occur as a result of Ras mutations as well as following other events that deregulate the pathway.

### EXAMPLE 4

### Detection of Pathway Deregulation in Lung Cancer with Hierarchical Clustering

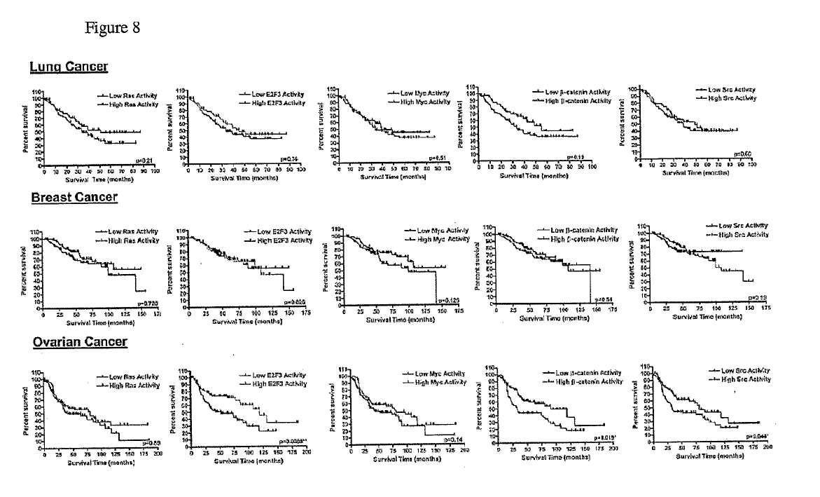

While the analysis of pathway deregulation as shown in FIG. 2C depicts the status of an individual pathway, the real power in this approach is the ability to identify patterns of pathway deregulation, using hierarchical clustering, much the same as identifying patterns of gene expression. An analysis of the lung cancer samples was done first (FIG. 3A, left panel). This analysis distinguished adenocarcinomas from squamous cell carcinomas, driven in part by the Ras pathway distinction. It is also evident that the tumors predicted as exhibiting relatively low Ras activity are generally predicted at higher levels of Myc, E2F3, β-catenin, and Src activity (clusters 1-3). Conversely, the tumors with relatively elevated Ras activity exhibited relatively lower levels of these other pathways (clusters 4-7). Independent of the tumor histopathology, concerted deregulation of Ras with β-catenin, Src, and Myc (cluster 8) identified a population of patients with poor survival—a median survival of 19.7 months vs. 51.3 months for all other clusters (FIG. 3A, right panel). Further, this subpopulation of patients exhibited worse survival than any of the groups of patients identified based on the status of any single pathway deregulation (FIG. 8). This analysis demonstrates the ability of integrated pathway analysis, based on multiple signatures of component pathway deregulation, to define improved categorization of lung cancer patients.

### EXAMPLE 5

### Detection of Pathway Deregulation in Breast and Ovarian Cancer with Hierarchical Clustering

Two additional examples made use of large sets of breast cancer samples (FIG. 3B) and ovarian cancer samples (FIG. 3C). Again, there were evident patterns of pathway deregulation, distinct from that seen in the lung samples, which characterized the breast and ovarian tumors. For breast cancer, clusters 2 and 3, which both contain ER positive tumors (and no discernable differences in Her2 status or other clinical parameters), show distinct survival rates (p value=0.07). Patients defined by cluster 5, in which higher than average β-catenin and Myc activities were predicted, and E2F3 activity was lower than average, exhibited very poor survival again illustrating the importance of co-deregulation of multiple oncogenic pathways as a determinant of clinical outcome. A final analysis made use of an advanced stage (III or IV) ovarian cancer dataset. The ovarian samples exhibited a dominant pattern of β-catenin and Src deregulation, either elevated (cluster 1 and 2) or diminished (clusters 3-6). Strikingly, the co-deregulation of Src and β-catenin defined by clusters 1 and 2 identifies a population of patients with very poor survival compared to other pathway clusters [median survival: 34.0 months vs. 112.0 months] (FIG. 3C, right panel). Once again, for these cases, individual pathway status did not stratify patient subgroups as effectively as patterns of multiple pathway deregulation (FIG. 8).

### EXAMPLE 6

### Detection of Pathway Deregulation to Predict Sensitivity to Therapeutic Agents

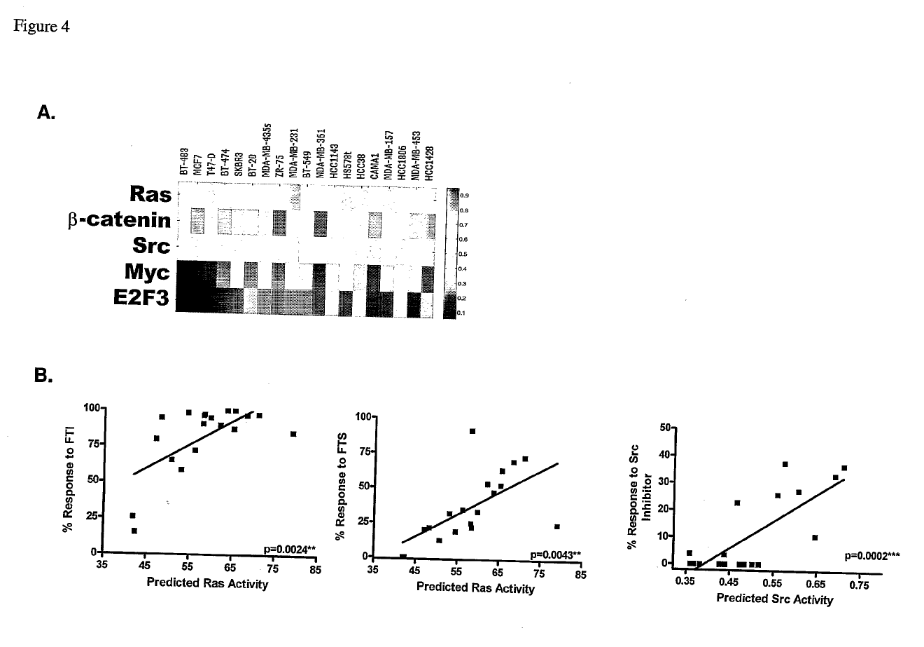

Given the capacity of the gene expression signatures to predict deregulation of oncogenic signaling pathways, the extent to which this could predict sensitivity to a therapeutic agent that targets that pathway is also addressed. To explore this, pathway deregulation was predicted in a series of breast cancer cell lines to be screened against potential therapeutic drugs. The results using the set of five pathway predictors, together with an initial collection of breast cancer cell lines, are reflected in FIG. 4A. Biochemical characteristics of the cell lines relevant for pathway analysis are summarized in Table 3, and FIG. 9. In each case, the relative probabilities of pathway activation are predicted from the signature in a manner completely analogous to the prediction of pathway status in tumors. In most cases, there is a good correlation between biochemical measures of pathway activation and prediction based on gene expression signatures. An exception is with Ras, where there is not a significant correlation between the biochemical measure of pathway activation and pathway prediction, presumably reflecting additional events not measured in the biochemical assay. Clearly, the critical issue is whether the gene expression signature predicts drug sensitivity—this point is addressed by the dose-response assays in FIG. 4B.

In parallel with mapping the pathway status, the cell lines were assayed with drugs known to target specific activities within given oncogenic pathways. The assays involve growth inhibition measurements using standard colorimetric assays^{17,18}. The result of testing sensitivity of the cell lines to inhibitors of the Ras pathway using both a farnesyl transferase inhibitor (L-744,832) and a farnesylthiosalicylic acid (FTS) is shown in FIG. 4B. In addition, a Src inhibitor (SU6656) was also employed for these assays. In each case, the results show a close concordance and correlation between the probability of Ras and Src pathway deregulation based on the gene expression prediction, and the extent of cell proliferation inhibition by the respective drugs (FIG. 4B). Furthermore, comparison of the drug inhibition results with predictions of other pathways failed to demonstrate a significant correlation (FIG. 10). These results confirm the ability of the defined “pathway deregulation signatures” to also predict sensitivity to therapeutic agents that target the corresponding pathways.

### EXAMPLE 7

### Methods

Cell and RNA preparation. Human mammary epithelial cells from a breast reduction surgery at Duke University were isolated and cultured according to previously published protocols²⁴. These cells were a generous gift from Gudrun Huper (Duke University). These cells are grown in MEBM (HEPES buffered) plus addition of a ‘bullet kit’ [Clonetics], and supplemented with 5 μg/ml transferrin and 10⁻⁵M isoproterenol at 3% CO₂. Cells are brought to quiescence by growing in 0.25% serum starvation media (without EGF) for 36 hours, and are then infected with (at 150 MOI) adenovirus expressing either human c-Myc, activated H-Ras, human c-Src, human E2F3, or activated β-catenin. Eighteen hours post-infection, cells are collected by scraping on ice in PBS and pelleting cells by centrifugation. Expression of oncogenes and their secondary targets was determined by a standard Western Blotting protocol using a TGH lysis buffer (1% Triton X-100, 10% glycerol, 50 mM NaCl, 50 mM Hepes, pH 7.3, 5 mM EDTA, 1 mM sodium orthovanadate, 1 mM PMSF, 10 μg/ml leupeptine, 10 μg/ml aprotinin). Lysates were rotated at 4° C. for 30 minutes and then centrifuged at 13,000×g for 30 minutes. Protein quantitation of lysates was determined by BCA [Pierce] prior to electrophoresis with a 10-12% SDS-PAGE gel. Activation status of kinase pathways for the breast cancer cell lines was determined for growing cells (at 75% confluency) 48 hours after plating using the following methods. Ras activation is measured using a Ras Activation Assay Kit (Upstate Biotechnology) that consists of a GST fusion-protein corresponding to the human Ras Binding Domain (RBD, residues 1-149) of Raf-1. The RBD specifically binds to and precipitates Ras-GTP from cell lysates. Western Blotting for immunoprecipitated H/K-Ras is detected using an H/K-Ras specific antibody (Santa Cruz Biotechnology, #sc-520 and sc-F234). c-Src activation was determined by Western Blotting using a phospho-Tyr416 Src antibody (Cell Signaling, #2101). E2F3, Myc, and β-catenin activity were measured by isolating nuclear extracts from cells as previously described, and performing Western Blotting analysis using antibodies for specific for E2F3, c-Myc, or β-catenin (Santa Cruz Biotechnology, sc-878, sc-42, sc-7199, respectively). Total RNA was extracted for cell lines using the Qiashredder and Qiagen Rneasy Mini kits. Quality of the RNA was checked by an Agilent 2100 Bioanalyzer.
Tumor analyses. Tumor tissue from breast, ovarian, and lung cancer patients were >60% tumor, and were selected for by stage and histology. Total RNA was extracted as previously described²⁰. Approximately 30 mg of tissue was added to a chilled BioPulverizer H tube [Bio101 Systems, Carlsbad, Calif.]. Lysis buffer from the Qiagen Rneasy Mini kit was added and the tissue homogenized for 20 seconds in a Mini-Beadbeater [Biospec Products, Bartlesville, Okla.]. Tubes were spun briefly to pellet the garnet mixture and reduce foam. The lysate was transferred to a new 1.5 ml tube using a syringe and 21 gauge needle, followed by passage through the needle 10 times to shear genomic DNA. Total RNA was extracted from tumors using the Qiagen Rneasy Mini kit. Quality of the RNA was checked by an Agilent 2100 Bioanalyzer.
DNA microarray analysis. Samples were prepared according to the manufacturer's instructions and as previously published^{21,22}. Experiments to generate signatures utilize Human U133 2.0 Plus GeneChips. Breast tumors were hybridized to Hu95Av2 arrays, ovarian tumors to Hu133A arrays, and lung tumors to Human U133 2.0 plus arrays [Affymetrix]. All microarray data is available at http://data.cgt.duke.edu/oncogene.php and on GEO. Labeled probes for Affymetrix DNA microarray analysis were prepared according to the manufacturer's instructions. Biotin-labeled cRNA, produced by in vitro transcription, was fragmented and hybridized to Affymetrix GeneChip arrays. Experiments to generate signatures utilize Human U133 2.0 Plus GeneChips. Tumor tissues were hybridized to various human Affymetrix GeneChip arrays, breast tumors were hybridized to Hu95Av2, ovarian tumors to Hu133A lung tumors to Human U133 2.0 plus array. DNA chips are scanned with the Affymetrix GeneChip scanner, and the signals are processed to evaluate the standard RMA measures of expression^{25,26}.
Cross-platform Affymetrix Gene Chip comparison. To map the probe sets across various generations of Affymetrix GeneChip arrays, we utilized an in-house program, Chip Comparer (http://tenero.duhs.duke.edu/genearray/perl/chip/chipcomparer.pl). First, each probeset ID in given Affymetrix gene chips were mapped to the corresponding LocusID. This is done by parsing local copies of LocusLink and UniGene databases to identify inherent relationship between the GenBank accession number associated with each probeset sequence and its corresponding LocusID. Second, probesets from different gene chips are matched by sharing the same LocusID (or orthologous pair of LocusIDs in the case of mapping gene chips across species).
Statistical analysis methods. Analysis of expression data are as previously described for¹² Prior to statistical modeling, gene expression data is filtered to exclude probesets with signals present at background noise levels, and for probesets that do not vary significantly across samples. A metagene represents a group of genes that together exhibit a consistent pattern of expression in relation to an observable phenotype. Each signature summarizes its constituent genes as a single expression profile, and is here derived as the first principal component of that set of genes (the factor corresponding to the largest singular value) as determined by a singular value decomposition. Given a training set of expression vectors (of values across metagenes) representing two biological states, a binary probit regression model is estimated using Bayesian methods. Applied to a separate validation data set, this leads to evaluations of predictive probabilities of each of the two states for each case in the validation set. When predicting the pathway activation of cancer cell lines or tumor samples, gene selection and identification is based on the training data, and then metagene values are computed using the principal components of the training data and additional cell line or tumor expression data. Bayesian fitting of binary probit regression models to the training data then permits an assessment of the relevance of the metagene signatures in within-sample classification, and estimation and uncertainty assessments for the binary regression weights mapping metagenes to probabilities of relative pathway status. Predictions of the relative pathway status of the validation cell lines or tumor samples are then evaluated, producing estimated relative probabilities—and associated measures of uncertainty—of activation/deregulation across the validation samples. Hierarchical clustering of tumor predictions was performed using Gene Cluster 3.0²⁷. Genes and tumors were clustered using average linkage with the uncentered correlation similarity metric. Standard Kaplan-Meier mortality curves and their significance were generated for clusters of patients with similar patterns of oncogenic pathway deregulation using GraphPad software. For the Kaplan-Meier survival analyses, the survival curves are compared using the logrank test. This test generates a two-tailed P value testing the null hypothesis, which is that the survival curves are identical in the overall populations. Therefore, the null hypothesis is that the populations have no differences in survival.
Cell proliferation assays. Sensitivity to a farnesyl transferase inhibitor (L-744,832), farnesylthiosalicylic acid (FTS), and a Src inhibitor (SU6656) was determined by quantifying the percent reduction in growth (versus DMSO controls) at 96 hrs using a standard MTT colorimetric assay. Concentrations used were from 100 nM-10 μM (L-744,832), 10-200 μM FTS, and 300 nM-10 μM (SU6656). Growth curves for the breast cancer cell lines profiled by gene array analyses was carried out by plating at 500-10,000 cells per well of a 96-well plate. The growth of cells at 12 hr time points (from t=12 hrs) was determined using the CellTiter 96 Aqueous One Solution Cell Proliferation Assay Kit by Promega, which is a calorimetric method for determining the number of growing cells. The growth curves plot the growth rate of cells on the Y-axis and time on the X-axis for each concentration of drug tested against each cell line. Cumulatively, these experiments determined the concentration of cells to use for each cell line, as well as the dosing range of the inhibitors (data not shown). The dose-response curves in our experiments plot the percent of cell population responding to the chemotherapy on the Y-axis and concentration of drug on the X-axis for each cell line. Sensitivity to a farnesyl transferase inhibitor (L-744,832), farnesylthiosalicylic acid (FTS), and a Src inhibitor (SU6656) was determined by quantifying the percent reduction in growth (versus DMSO controls) at 96 hrs. Concentrations used were from 100 nM-10 μM (L-744,832), 10-200 μM FTS, and 300 nM-10 μM (SU6656). All experiments were repeated at least three times.
K-Ras mutation assay. K-Ras mutation status was determined using restriction fragment length polymorphism and sequencing as previously described²⁴ Tumor DNA was isolated as described and 100 ng of genomic DNA was amplified in a volume of 100 μl as described [Mitsudomi 1991]. At codon 12 of the K-ras gene, a Ban1 restriction site is introduced by inserting a C residue at the second position of codon 13 using a mismatched primer K12ABan (SEQ ID NO. 1) (5′-CAAGGCACTCTTGCCTACGGC-3′). Any mutation at codon 12 will abolish the Ban1 restriction site. Restriction enzyme digestion was carried out overnight at 37°. Restriction products were isolated by gel electrophoresis with a 4% low melting agarose gel. Unrestricted bands indicative of a point mutation in codon 12 were isolated and sequenced for verification.

SUPPLEMENTAL TABLE 1
Genes that constitute pathway signatures.

| ProbeID | GeneSymbol | Description | LocusLink | Fold Ch [symbol] |
|---|---|---|---|---|
| Myc | | | | |
| 208161_s_at | ABCC3 | ATP-binding cassette, sub-family C (CFTR/MRP), member 3 | 8714 | 0.619311 |
| 209641_s_at | ABCC3 | ATP-binding cassette, sub-family C (CFTR/MRP), member 3 | 8714 | 0.58333 [symbol] |
| 231907_at | ABL2 | V-abl Abelson murine leukemia viral oncogene homolog 2 (arg, Abelson-related gene) | 27 | 0.80770 [symbol] |
| 234312_s_at | ACAS2 | Acetyl-Coenzyme A synthetase 2 (ADP forming) | 55902 | 0.77657 [symbol] |
| 205180_s_at | ADAM8 | A disintegrin and metalloproteinase domain 8 | 101 | 0.689631 |
| 227530_at | AKAP12 | A kinase (PRKA) anchor protein (gravin) 12 | 9590 | 0.51322 [symbol] |
| 227529_s_at | AKAP12 | A kinase (PRKA) anchor protein (gravin) 12 | 9590 | 0.35218 [symbol] |
| 209645_s_at | ALDH1B1 | Aldehyde dehydrogenase 1 family, member B1 | 219 | 1.26867 [symbol] |
| 207396_s_at | ALG3 | Asparagine-linked glycosylation 3 homolog (yeast, alpha-1,3-mannosyltransferase) | 10195 | 1.91928 [symbol] |
| 229267_at | ANAPC1 | Anaphase promoting complex subunit 1 | 64682 | 1.31745 [symbol] |
| 224634_at | APOA1BP | Apolipoprotein A-I binding protein | 128240 | 1.61371 [symbol] |
| 47069_at | ARHGAP8 | Data not found | 23779 | 1.18668 [symbol] |
| 209824_s_at | ARNTL | Aryl hydrocarbon receptor nuclear translocator-like | 406 | 0.44197 [symbol] |
| 210971_s_at | ARNTL | Aryl hydrocarbon receptor nuclear translocator-like | 406 | 0.45015 [symbol] |
| 224204_x_at | ARNTL2 | Aryl hydrocarbon receptor nuclear translocator-like 2 | 56938 | 0.61516 [symbol] |
| 208758_at | ATIC | 5-aminoimidazole-4-carboxamide ribonucleotide formyltransferase/IMP cyclohydrolase | 471 | 1.57154 [symbol] |
| 212135_s_at | ATP2B4 | Data not found | 493 | 0.61366 [symbol] |
| 205410_s_at | ATP2B4 | Data not found | 493 | 0.57777 [symbol] |
| 207618_s_at | BCS1L | BCS1-like (yeast) | 617 | 1.16467 [symbol] |
| 220688_s_at | C1orf33 | Chromosome 1 open reading frame 33 | 51154 | 1.85532 [symbol] |
| 50314_i_at | C20orf27 | Chromosome 20 open reading frame 27 | 54976 | 1.75233 [symbol] |
| 211559_s_at | CCNG2 | Cyclin G2 | 901 | 0.56603 [symbol] |
| 221520_s_at | CDCA8 | Cell division cycle associated 8 | 55143 | 0.54574 [symbol] |
| 211804_s_at | CDK2 | Cyclin-dependent kinase 2 | 1017 | 0.28796 [symbol] |
| 202246_s_at | CDK4 | Cyclin-dependent kinase 4 | 1019 | 1.61359 [symbol] |
| 211862_x_at | CFLAR | CASP8 and FADD-like apoptosis regulator | 8837 | 0.76210 [symbol] |
| 218732_at | CGI-147 | Bcl-2 inhibitor of transcription | 51651 | 1.81893 [symbol] |
| 223232_s_at | CGN | Cingulin | 57530 | 0.62387 [symbol] |
| 230656_s_at | CIRH1A | Cirrhosis, autosomal recessive 1A (cirhin) | 84916 | 1.66355 [symbol] |
| 224903_at | CIRH1A | Cirrhosis, autosomal recessive 1A (cirhin) | 84916 | 1.62898 [symbol] |
| 233986_s_at | CLG | Pleckstrin homology domain containing, family G (with RhoGef domain) member 2 | 64857 | 0.24464 [symbol] |
| 202310_s_at | COL1A1 | Collagen, type I, alpha 1 | 1277 | 0.59446 [symbol] |
| 203325_s_at | COL5A1 | Collagen, type V, alpha 1 | 1289 | 0.67295 [symbol] |
| 221900_at | COL8A2 | Collagen, type VIII, alpha 2 | 1296 | 0.80192 [symbol] |
| 205076_s_at | CRA | Myotubularin related protein 11 | 10903 | 0.62691 [symbol] |
| 215537_x_at | DDAH2 | Dimethylarginine dimethylaminohydrolase 2 | 23564 | 0.693711 |
| 202262_x_at | DDAH2 | Dimethylarginine dimethylaminohydrolase 2 | 23564 | 0.42244 [symbol] |
| 204977_at | DDX10 | DEAD (Asp-Glu-Ala-Asp) box polypeptide 10 | 1662 | 1.83382 [symbol] |
| 208895_s_at | DDX18 | DEAD (Asp-Glu-Ala-Asp) box polypeptide 18 | 8886 | 1.43017 [symbol] |
| 203385_at | DGKA | Diacylglycerol kinase, alpha 80 kDa | 1606 | 0.77032 [symbol] |
| 213632_at | DHODH | Dihydroorotate dehydrogenase | 1723 | 1.47680 [symbol] |
| 213279_at | DHRS1 | Dehydrogenase/reductase (SDR family) member 1 | 115817 | 0.69694 [symbol] |
| 201479_at | DKC1 | Dyskeratosis congenita 1, dyskerin | 1736 | 2.03138 [symbol] |
| 226763_at | DKFZp434O0515 | SEC14 and spectrin domains 1 | 91404 | 0.71892 [symbol] |
| 209725_at | DRIM | Down-regulated in metastasis | 27340 | 1.91234 [symbol] |
| 215800_at | DUOX1 | Dual oxidase 1 | 53905 | 0.86276 [symbol] |
| 204794_at | DUSP2 | Dual specificity phosphatase 2 | 1844 | 6.98197 [symbol] |
| 226440_at | DUSP22 | Dual specificity phosphatase 22 | 56940 | 0.73396 [symbol] |
| 201325_s_at | EMP1 | Epithelial membrane protein 1 | 2012 | 0.60702 [symbol] |
| 91826_at | EPS8L1 | EPS8-like 1 | 54869 | 0.72091 [symbol] |
| 218779_x_at | EPS8L1 | EPS8-like 1 | 54869 | 0.73432 [symbol] |
| 226213_at | ERBB3 | V-erb-b2 erythroblastic leukemia viral oncogene homolog 3 (avian) | 2065 | 0.68150 [symbol] |
| 228131_at | ERCC1 | Excision repair cross-complementing rodent repair deficiency, complementation group 1 | 2067 | 0.744781 |
| 202159_at | FARSL | Phenylalanine-tRNA synthetase-like, alpha subunit | 2193 | 1.54465 [symbol] |
| 226799_at | FGD6 | FYVE, RhoGEF and PH domain containing 6 | 55785 | 0.55730 [symbol] |
| 227271_at | FGF11 | Fibroblast growth factor 11 | 2256 | 0.90006 [symbol] |
| 226698_at | FLJ00007 | FCH and double SH3 domains 1 | 89848 | 0.836111 |
| 218920_at | FLJ10404 | Hypothetical protein FLJ10404 | 54540 | 0.78984 [symbol] |
| 221712_s_at | FLJ10439 | Hypothetical protein FLJ10439 | 54663 | 1.50293 [symbol] |
| 203867_s_at | FLJ10458 | Notchless gene homolog (Drosophila) | 54475 | 1.78078 [symbol] |
| 220353_at | FLJ10661 | Data not found | 55199 | 1.23397 [symbol] |
| 221536_s_at | FLJ11301 | Hypothetical protein FLJ11301 | 55341 | 1.41816 [symbol] |
| 223200_s_at | FLJ11301 | Hypothetical protein FLJ11301 | 55341 | 1.60441 [symbol] |
| 219987_at | FLJ12684 | Hypothetical protein FLJ12684 | 79584 | 2.11148 [symbol] |
| 236635_at | FLJ14011 | Zinc finger protein 667 | 63934 | 1.71399 [symbol] |
| 210463_x_at | FLJ20244 | Hypothetical protein FLJ20244 | 55621 | 2.18767 [symbol] |
| 203701_s_at | FLJ20244 | Hypothetical protein FLJ20244 | 55621 | 1.66066 [symbol] |
| 203785_s_at | FLJ20399 | Dihydrouridine synthase 2-like (SMM1, S. cerevisiae) | 54920 | 2.54545 [symbol] |
| 235026_at | FLJ32549 | Hypothetical protein FLJ32549 | 144577 | 2.93590 [symbol] |
| 236745_at | FLJ34512 | Hypothetical protein FLJ34512 | 124093 | 2.17176 [symbol] |
| 222333_at | FLJ36525 | ALS2 C-terminal like | 259173 | 0.71815 [symbol] |
| 223035_s_at | FRSB | Phenylalanine-tRNA synthetase-like, beta subunit | 10056 | 2.20072 [symbol] |
| 225712_at | GEMIN5 | Gem (nuclear organelle) associated protein 5 | 25929 | 2.74622 [symbol] |
| 35436_at | GOLGA2 | Golgi autoantigen, golgin subfamily a, 2 | 2801 | 0.69156 [symbol] |
| 238689_at | GPR110 | G protein-coupled receptor 110 | 266977 | 0.50815 [symbol] |
| 205014_at | HBP17 | Fibroblast growth factor binding protein 1 | 9982 | 0.66725 [symbol] |
| 222305_at | HK2 | Hexokinase 2 | 3099 | 2.02173 [symbol] |
| 209971_x_at | HRI | Eukaryotic translation initiation factor 2-alpha kinase 1 | 27102 | 1.59785 [symbol] |
| 1552334_at | HRIHFB2122 | Tara-like protein | 11078 | 0.59303 [symbol] |
| 1552767_a_at | HS6ST2 | Heparan sulfate 6-O-sulfotransferase 2 | 90161 | 2.18211 [symbol] |
| 200800_s_at | HSPA1A | Heat shock 70 kDa protein 1A | 3303 | 3.14524 [symbol] |
| 213418_at | HSPA6 | Heat shock 70 kDa protein 6 (HSP70B′) | 3310 | 12.03537 |
| 214011_s_at | HSPC111 | Hypothetical protein HSPC111 | 51491 | 1.56933 [symbol] |
| 200807_s_at | HSPD1 | Heat shock 60 kDa protein 1 (chaperonin) | 3329 | 1.59802 [symbol] |
| 212411_at | IMP4 | IMP4, U3 small nucleolar ribonucleoprotein, homolog (yeast) | 92856 | 1.41289 [symbol] |
| 218305_at | IPO4 | Importin 4 | 79711 | 1.646651 |
| 203882_at | ISGF3G | Interferon-stimulated transcription factor 3, gamma 48 kDa | 10379 | 0.674311 |
| 202138_x_at | JTV1 | JTV1 gene | 7965 | 1.55906 [symbol] |
| 212510_at | KIAA0089 | Glycerol-3-phosphate dehydrogenase 1-like | 23171 | 2.06513 [symbol] |
| 1552257_a_at | KIAA0153 | KIAA0153 protein | 23170 | 1.37496 [symbol] |
| 212357_at | KIAA0280 | KIAA0280 protein | 23201 | 0.71496 [symbol] |
| 212356_at | KIAA0323 | KIAA0323 | 23351 | 0.79605 [symbol] |
| 212355_at | KIAA0323 | KIAA0323 | 23351 | 0.78451 [symbol] |
| 36865_at | KIAA0759 | KIAA0759 | 23357 | 1.44603 [symbol] |
| 227920_at | KIAA1553 | KIAA1553 | 57673 | 1.34277 [symbol] |
| 225929_s_at | KIAA1554 | Chromosome 17 open reading frame 27 | 57674 | 0.75958 [symbol] |
| 221843_s_at | KIAA1609 | KIAA1609 protein | 57707 | 0.74631 [symbol] |
| 207517_at | LAMC2 | Laminin, gamma 2 | 3918 | 0.61855 [symbol] |
| 225874_at | LOC124402 | LOC124402 | 124402 | 1.53552 [symbol] |
| 227285_at | LOC148523 | Chromosome 1 open reading frame 51 | 148523 | 1.51884 [symbol] |
| 227037_at | LOC201164 | Similar to CG12314 gene product | 201164 | 2.11556 [symbol] |
| 227485_at | LOC203522 | DEAD/H (Asp-Glu-Ala-Asp/His) box polypeptide 26B | 203522 | 0.72316 [symbol] |
| 218096_at | LPAAT-e | 1-acylglycerol-3-phosphate O-acyltransferase 5 (lysophosphatidic acid | 55326 | 2.23867 [symbol] |
| | | acyltransferase, epsilon) | | |
| 204682_at | LTBP2 | Latent transforming | 4053 | 0.75924 [symbol] |
| | | growth factor beta binding protein 2 | | |
| 212281_s_at | MAC30 | Hypothetical protein MAC30 | 27346 | 2.73674 [symbol] |
| 212282_at | MAC30 | Hypothetical protein MAC30 | 27346 | 2.24042 [symbol] |
| 212279_at | MAC30 | Hypothetical protein MAC30 | 27346 | 2.084171 |
| 219278_at | MAP3K6 | Mitogen-activated protein kinase kinase kinase 6 | 9064 | 0.57026 [symbol] |
| 230110_at | MCOLN2 | Mucolipin 2 | 255231 | 1.38479 [symbol] |
| 226211_at | MEG3 | maternally expressed 3 | 55384 | 0.64528 [symbol] |
| 226210_s_at | MEG3 | maternally expressed 3 | 55384 | 0.56798 [symbol] |
| 204027_s_at | METTL1 | Methyltransferase like 1 | 4234 | 1.84529 [symbol] |
| 232077_s_at | MGC10500 | Yippee-like 3 (Drosophila) | 83719 | 0.38060 [symbol] |
| 224468_s_at | MGC13170 | Multidrug resistance-related protein | 84798 | 2.02223 [symbol] |
| 224500_s_at | MGC13272 | MON1 homolog A (yeast) | 84315 | 1.64247 [symbol] |
| 1553715_s_at | MGC15416 | Hypothetical protein MGC15416 | 84331 | 1.57578 [symbol] |
| 227103_s_at | MGC2408 | Data not found | 84291 | 2.37098 [symbol] |
| 221637_s_at | MGC2477 | Hypothetical protein MGC2477 | 79081 | 1.49234 [symbol] |
| 203119_at | MGC2574 | Hypothetical protein MGC2574 | 79080 | 1.66001 [symbol] |
| 204699_s_at | MGC29875 | Hypothetical protein MGC29875 | 27042 | 1.51920 [symbol] |
| 218953_s_at | MGC3265 | Hypothetical protein MGC3265 | 78991 | 1.46220 [symbol] |
| 211986_at | MGC5395 | AHNAK nucleoprotein (desmoyokin) | 79026 | 0.64109 [symbol] |
| 235281_x_at | MGC5395 | AHNAK nucleoprotein (desmoyokin) | 79026 | 0.56654 [symbol] |
| 209467_s_at | MKNK1 | MAP kinase interacting serine/threonine kinase 1 | 8569 | 0.72660 [symbol] |
| 205455_at | MST1R | Macrophage stimulating 1 receptor (c-met-related tyrosine kinase) | 4486 | 0.70208 [symbol] |
| 233803_s_at | MYBBP1A | MYB binding protein (P160) 1a | 10514 | 2.19495 [symbol] |
| 202431_s_at | MYC | V-myc myelocytomatosis viral oncogene homolog (avian) | 4609 | 4.64893 [symbol] |
| 211824_x_at | NALP1 | NACHT, leucine rich repeat and PYD (pyrin domain) containing 1 | 22861 | 0.51592 [symbol] |
| 211822_s_at | NALP1 | NACHT, leucine rich repeat and PYD (pyrin domain) containing 1 | 22861 | 0.58243 [symbol] |
| 200610_s_at | NCL | Nucleolin | 4691 | 2.16039 [symbol] |
| 227249_at | NDE1 | NudE nuclear distribution gene E homolog 1 (A. nidulans) | 54820 | 0.70665 [symbol] |
| 207535_s_at | NFKB2 | Nuclear factor of kappa light polypeptide gene enhancer in B-cells 2 (p49/p100) | 4791 | 0.70907 [symbol] |
| 205858_at | NGFR | Nerve growth factor receptor (TNFR superfamily, member 16) | 4804 | 0.57761 [symbol] |
| 218376_s_at | NICAL | Microtubule associated monoxygenase, calponin and LIM domain containing 1 | 64780 | 0.52968 [symbol] |
| 202891_at | NIT1 | Nitrilase 1 | 4817 | 0.732601 |
| 214427_at | NOL1 | Nucleolar protein 1, 120 kDa | 4839 | 1.23199 [symbol] |
| 200875_s_at | NOL5A | Nucleolar protein 5A (56 kDa with KKE/D repeat) | 10528 | 2.03470 [symbol] |
| 218199_s_at | NOL6 | Nucleolar protein family 6 (RNA-associated) | 65083 | 1.86172 [symbol] |
| 211951_at | NOLC1 | Nucleolar and coiled-body phosphoprotein 1 | 9221 | 1.90580 [symbol] |
| 205895_s_at | NOLC1 | Nucleolar and coiled-body phosphoprotein 1 | 9221 | 1.44239 [symbol] |
| 200063_s_at | NPM1 | Nucleophosmin (nucleolar phosphoprotein B23, numatrin) | 4869 | 1.36883 [symbol] |
| 212298_at | NRP1 | Neuropilin 1 | 8829 | 0.50802 [symbol] |
| 217850_at | NS | Guanine nucleotide binding protein-like 3 (nucleolar) | 26354 | 1.76404 [symbol] |
| 231785_at | NTF5 | Neurotrophin 5 (neurotrophin 4/5) | 4909 | 0.48850 [symbol] |
| 206376_at | NTT73 | Solute carrier family 6, member 15 | 55117 | 2.68720 [symbol] |
| 239352_at | NTT73 | Solute carrier family 6, member 15 | 55117 | 1.96673 [symbol] |
| 205135_s_at | NUFIP1 | Nuclear fragile X mental retardation protein interacting protein 1 | 26747 | 1.65565 [symbol] |
| 223432_at | OSBP2 | Oxysterol binding protein 2 | 23762 | 0.46825 [symbol] |
| 208676_s_at | PA2G4 | proliferation-associated 2G4, 38 kDa | 5036 | 1.52190 [symbol] |
| 201013_s_at | PAICS | Phosphoribosylaminoimidazole carboxylase, phosphoribosylaminoimidazole | 10606 | 1.84577 [symbol] |
| | | succinocarboxamide syntheta [symbol] | | |
| 204476_s_at | PC | Pyruvate carboxylase | 5091 | 0.45672 [symbol] |
| 219295_s_at | PCOLCE2 | Procollagen C-endopeptidase enhancer 2 | 26577 | 1.93576 [symbol] |
| 218590_at | PEO1 | Progressive external ophthalmoplegia 1 | 56652 | 2.07225 [symbol] |
| 202212_at | PES1 | Pescadillo homolog 1, containing BRCT domain (zebrafish) | 23481 | 1.94481 [symbol] |
| 210976_s_at | PFKM | Phosphofructokinase, muscle | 5213 | 1.54026 [symbol] |
| 200658_s_at | PHB | Prohibitin | 5245 | 1.57996 [symbol] |
| 40446_at | PHF1 | Data not found | 5252 | 0.57520 [symbol] |
| 211668_s_at | PLAU | Data not found | 5328 | 0.48390 [symbol] |
| 201373_at | PLEC1 | Plectin 1, intermediate filament binding protein 500 kDa | 5339 | 0.64357 [symbol] |
| 203201_at | PMM2 | Phosphomannomutase 2 | 5373 | 1.76150 [symbol] |
| 225291_at | PNPT1 | Polyribonucleotide nucleotidyltransferase 1 | 87178 | 1.39737 [symbol] |
| 212541_at | PP591 | FAD-synthetase | 80308 | 1.66864 [symbol] |
| 218273_s_at | PPM2C | Protein phosphatase 2C, magnesium-dependent, catalytic subunit | 54704 | 0.61809 [symbol] |
| 209158_s_at | PSCD2 | Data not found | 9266 | 0.85492 [symbol] |
| 203150_at | RAB9P40 | Rab9 effector p40 | 10244 | 1.30987 [symbol] |
| 203108_at | RAI3 | G protein-coupled receptor, family C, group 5, member A | 9052 | 0.35620 [symbol] |
| 212444_at | RAI3 | G protein-coupled receptor, family C, group 5, member A | 9052 | 0.39148 [symbol] |
| 222666_s_at | RCL1 | RNA terminal phosphate cyclase-like 1 | 10171 | 1.889821 |
| 218686_s_at | RHBDF1 | Rhomboid family 1 (Drosophila) | 64285 | 0.74774 [symbol] |
| 213427_at | RNASEP1 | Ribonuclease P 40 kDa subunit | 10799 | 2.03728 [symbol] |
| 224610_at | RNU22 | RNA, U22 small nucleolar | 9304 | 1.60486 [symbol] |
| 204133_at | RNU3IP2 | RNA, U3 small nucleolar interacting protein 2 | 9136 | 2.90361 [symbol] |
| 218481_at | RRP46 | Exosome component 5 | 56915 | 2.04571 [symbol] |
| 210365_at | RUNX1 | Runt-related transcription factor 1 (acute myeloid leukemia 1; aml1 oncogene) | 861 | 0.55607 [symbol] |
| 230333_at | SAT | Spermidine/spermine N1-acetyltransferase | 6303 | 0.53083 [symbol] |
| 221514_at | SDCCAG16 | UTP14, U3 small nucleolar ribonucleoprotein, homolog A (yeast) | 10813 | 2.20107 [symbol] |
| 221513_s_at | SDCCAG16 | UTP14, U3 small nucleolar ribonucleoprotein, homolog A (yeast) | 10813 | 1.488051 |
| 212268_at | SERPINB1 | Serine (or cysteine) proteinase inhibitor, clade B (ovalbumin), member 1 | 1992 | 0.474371 |
| 225143_at | SFXN4 | Sideroflexin 4 | 119559 | 1.59110 [symbol] |
| 229236_s_at | SFXN4 | Sideroflexin 4 | 119559 | 1.44758 [symbol] |
| 219874_at | SLC12A8 | Solute carrier family 12 (potassium/chloride transporters), member 8 | 84561 | 1.92208 [symbol] |
| 211576_s_at | SLC19A1 | Solute carrier family 19 (folate transporter), member 1 | 6573 | 2.03331 [symbol] |
| 209776_s_at | SLC19A1 | Solute carrier family 19 (folate transporter), member 1 | 6573 | 3.119031 |
| 204717_s_at | SLC29A2 | Solute carrier family 29 (nucleoside transporters), member 2 | 3177 | 1.61512 [symbol] |
| 202219_at | SLC6A8 | Solute carrier family 6 (neurotransmitter transporter, creatine), member 8 | 6535 | 2.40855 [symbol] |
| 232481_s_at | SLITRK6 | SLIT and NTRK-like family, member 6 | 84189 | 0.62637 [symbol] |
| 207390_s_at | SMTN | Smoothelin | 6525 | 0.64228 [symbol] |
| 209427_at | SMTN | Smoothelin | 6525 | 0.57902 [symbol] |
| 212666_at | SMURF1 | SMAD specific E3 ubiquitin protein ligase 1 | 57154 | 0.60275 [symbol] |
| 201563_at | SORD | Sorbitol dehydrogenase | 6652 | 1.95231 [symbol] |
| 203509_at | SORL1 | Data not found | 6653 | 0.68312 [symbol] |
| 215235_at | SPTAN1 | Spectrin, alpha, non-erythrocytic 1 (alpha-fodrin) | 6709 | 0.69527 [symbol] |
| 208611_s_at | SPTAN1 | Spectrin, alpha, non-erythrocytic 1 (alpha-fodrin) | 6709 | 0.69231 [symbol] |
| 229952_at | SPTB | Spectrin, beta, erythrocytic (includes spherocytosis, clinical type I) | 6710 | 0.518651 |
| 201516_at | SRM | Spermidine synthase | 6723 | 1.93966 [symbol] |
| 51192_at | SSH-3 | Slingshot homolog 3 (Drosophila) | 54961 | 0.78523 [symbol] |
| 222557_at | STMN3 | Stathmin-like 3 | 50861 | 0.72347 [symbol] |
| 226923_at | STXBP1L1 | Sec1 family domain containing 2 | 152579 | 1.72478 [symbol] |
| 212894_at | SUPV3L1 | Suppressor of var1, 3-like 1 (S. cerevisiae) | 6832 | 1.39686 [symbol] |
| 235020_at | TAF4B | TAF4b RNA polymerase II, TATA box binding protein (TBP)-associated factor, 105 kDa | 6875 | 2.07508 [symbol] |
| 202384_s_at | TCOF1 | Treacher Collins-Franceschetti syndrome 1 | 6949 | 1.47214 [symbol] |
| 219131_at | TERE1 | Transitional epithelia response protein | 29914 | 2.58880 [symbol] |
| 218605_at | TFB2M | Transcription factor B2, mitochondrial | 64216 | 1.86729 [symbol] |
| 206008_at | TGM1 | Transglutaminase 1 (K polypeptide epidermal type I, | 7051 | 0.47836 [symbol] |
| | | protein-glutamine-gamma-glutamyltransferase) | | |
| 223776_x_at | TINF2 | TERF1 (TRF1)-interacting nuclear factor 2 | 26277 | 0.81784 [symbol] |
| 202510_s_at | TNFAIP2 | Tumor necrosis factor, alpha-induced protein 2 | 7127 | 0.57931 [symbol] |
| 209118_s_at | TUBA3 | Tubulin, alpha 3 | 7846 | 0.49901 [symbol] |
| 213326_at | VAMP1 | Vesicle-associated membrane protein 1 (synaptobrevin 1) | 6843 | 0.602631 |
| 1569003_at | VMP1 | Transmembrane protein 49 | 81671 | 0.64108 [symbol] |
| 224917_at | VMP1 | Transmembrane protein 49 | 81671 | 0.46742 [symbol] |
| 218512_at | WDR12 | WD repeat domain 12 | 55759 | 1.72013 [symbol] |
| 226938_at | WDR21 | WD repeat domain 21A | 26094 | 1.74754 [symbol] |
| 201294_s_at | WSB1 | WD repeat and SOCS box-containing 1 | 26118 | 0.60239 [symbol] |
| 223055_s_at | XPO5 | Exportin 5 | 57510 | 1.50960 [symbol] |
| 219836_at | ZBED2 | Zinc finger, BED domain containing 2 | 79413 | 0.49262 [symbol] |
| 222227_at | ZNF236 | Zinc finger protein 236 | 7776 | 0.00438 [symbol] |
| 117_at | — | Data not found | — | 4.01548 [symbol] |
| 244623_at | — | Data not found | — | 2.49491 [symbol] |
| 229715_at | — | Data not found | — | 2.32299 [symbol] |
| 65585_at | — | Data not found | — | 2.03424 [symbol] |
| 1562904_s_at | — | Similar to hypothetical protein SB153 isoform 1 | 286042 | 2.22325 [symbol] |
| 212563_at | — | Data not found | — | 1.65756 [symbol] |
| 234049_at | — | Similar to hypothetical protein SB153 isoform 1 | 286042 | 4.38431 [symbol] |
| 216212_s_at | — | Data not found | — | 6.10412 [symbol] |
| 211725_s_at | — | Data not found | — | 1.54287 [symbol] |
| 1556111_s_at | — | Data not found | — | 1.77764 [symbol] |
| 224603_at | — | Data not found | — | 1.46760 [symbol] |
| 1568597_at | — | Data not found | — | 1.40867 [symbol] |
| 235474_at | — | Data not found | — | 1.54637 [symbol] |
| 225933_at | — | Data not found | 339230 | 1.31950 [symbol] |
| 241687_at | — | Data not found | — | 1.64888 [symbol] |
| 202632_at | — | Data not found | — | 1.19481 [symbol] |
| 235501_at | — | Data not found | — | 0.88599 [symbol] |
| 65521_at | — | Data not found | — | 0.77884 [symbol] |
| 233493_at | — | Data not found | 377582 | 0.71695 [symbol] |
| 179_at | — | Data not found | — | 0.78843 [symbol] |
| 201278_at | — | Data not found | — | 0.78806 [symbol] |
| 1555673_at | — | Data not found | — | 0.61992 [symbol] |
| 201042_at | — | Data not found | — | 0.56196 [symbol] |
| 237591_at | — | Data not found | — | 0.60593 [symbol] |
| 1562416_at | — | Data not found | — | 0.70024 [symbol] |
| 238967_at | — | Data not found | — | 0.57523 [symbol] |
| 229004_at | — | Data not found | — | 0.55836 [symbol] |
| 216971_s_at | — | Data not found | — | 0.54685 [symbol] |
| 242509_at | — | Data not found | — | 0.53339 [symbol] |
| 1569150_x_at | — | Data not found | — | 0.53408 [symbol] |
| 215071_s_at | — | Data not found | — | 0.43425 [symbol] |
| 1568408_x_at | — | Data not found | — | 0.601921 |
| E2F3 | | | | |
| 223320_s_at | ABCB10 | ATP-binding cassette, sub-family B (MDR/TAP), member 10 | 23456 | 1.84854 [symbol] |
| 213485_s_at | ABCC10 | ATP-binding cassette, sub-family C (CFTR/MRP), member 10 | 89845 | 0.66003 [symbol] |
| 209735_at | ABCG2 | ATP-binding cassette, sub-family G (WHITE), member 2 | 9429 | 3.59315 [symbol] |
| 239579_at | ABHD7 | Abhydrolase domain containing 7 | 253152 | 3.72835 [symbol] |
| 209321_s_at | ADCY3 | Adenylate cyclase 3 | 109 | 1.65526 [symbol] |
| 218697_at | AF3P21 | NCK interacting protein with SH3 domain | 51517 | 1.32976 [symbol] |
| 225342_at | AK3 | Data not found | 205 | 1.75971 [symbol] |
| 201272_at | AKR1B1 | Aldo-keto reductase family 1, member B1 (aldose reductase) | 231 | 1.45332 [symbol] |
| 207163_s_at | AKT1 | V-akt murine thymoma viral oncogene homolog 1 | 207 | 1.66245 [symbol] |
| 203608_at | ALDH5A1 | Aldehyde dehydrogenase 5 family, member A1 (succinate-semialdehyde dehydrogenase) | 7915 | 2.90374 [symbol] |
| 223094_s_at | ANKH | Ankylosis, progressive homolog (mouse) | 56172 | 1.53787 [symbol] |
| 228415_at | AP1S2 | Adaptor-related protein complex 1, sigma 2 subunit | 8905 | 1.458561 |
| 239435_x_at | APXL2 | Apical protein 2 | 134549 | 1.84404 [symbol] |
| 37117_at | ARHGAP8 | Data not found | 23779 | 0.66463 [symbol] |
| 205980_s_at | ARHGAP8 | Data not found | 23779 | 0.72631 [symbol] |
| 235333_at | B4GALT6 | UDP-Gal:betaGlcNAc beta 1,4-galactosyltransferase, polypeptide 6 | 9331 | 1.91404 [symbol] |
| 204966_at | BAI2 | Brain-specific angiogenesis inhibitor 2 | 576 | 3.40317 [symbol] |
| 225606_at | BCL2L11 | BCL2-like 11 (apoptosis facilitator) | 10018 | 1.90208 [symbol] |
| 223566_s_at | BCOR | BCL6 co-repressor | 54880 | 1.77815 [symbol] |
| 219433_at | BCOR | BCL6 co-repressor | 54880 | 2.199221 |
| 231810_at | BRI3BP | BRI3 binding protein | 140707 | 2.62905 [symbol] |
| 225224_at | C20orf112 | Chromosome 20 open reading frame 112 | 140688 | 2.18004 [symbol] |
| 218796_at | C20orf42 | Chromosome 20 open reading frame 42 | 55612 | 0.66132 [symbol] |
| 227456_s_at | C6orf136 | Chromosome 6 open reading frame 136 | 221545 | 1.40648 [symbol] |
| 227455_at | C6orf136 | Chromosome 6 open reading frame 136 | 221545 | 1.78753 [symbol] |
| 232067_at | C6orf168 | Chromosome 6 open reading frame 168 | 84553 | 5.190981 |
| 221766_s_at | C6orf37 | Family with sequence similarity 46, member A | 55603 | 1.53675 [symbol] |
| 218309_at | CaMKIINalpha | Calcium/calmodulin-dependent protein kinase II | 55450 | 2.07720 [symbol] |
| 212252_at | CAMKK2 | Calcium/calmodulin-dependent protein kinase kinase 2, beta | 10645 | 1.44208 [symbol] |
| 201700_at | CCND3 | Cyclin D3 | 896 | 1.848871 |
| 213523_at | CCNE1 | Cyclin E1 | 898 | 6.06740 [symbol] |
| 211814_s_at | CCNE2 | Data not found | 9134 | 4.60598 [symbol] |
| 205034_at | CCNE2 | Data not found | 9134 | 12.1329 [symbol] |
| 204440_at | CD83 | CD83 antigen (activated B lymphocytes, immunoglobulin superfamily) | 9308 | 6.57980 [symbol] |
| 212899_at | CDK11 | Cell division cycle 2-like 6 (CDK8-like) | 23097 | 2.19008 [symbol] |
| 212897_at | CDK11 | Cell division cycle 2-like 6 (CDK8-like) | 23097 | 1.60031 [symbol] |
| 219534_x_at | CDKN1C | Cyclin-dependent kinase inhibitor 1C (p57, Kip2) | 1028 | 4.51403 [symbol] |
| 209644_x_at | CDKN2A | Data not found | 1029 | 1.29643 [symbol] |
| 204159_at | CDKN2C | Cyclin-dependent kinase inhibitor 2C (p18, inhibits CDK4) | 1031 | 7.65618 [symbol] |
| 204039_at | CEBPA | CCAAT/enhancer binding protein (C/EBP), alpha | 1050 | 4.37706 [symbol] |
| 205567_at | CHST1 | Carbohydrate (keratan sulfate Gal-6) sulfotransferase 1 | 8534 | 2.37735 [symbol] |
| 203921_at | CHST2 | Carbohydrate (N-acetylglucosamine-6-O) sulfotransferase 2 | 9435 | 2.267341 |
| 206756_at | CHST7 | Carbohydrate (N-acetylglucosamine 6-O) sulfotransferase 7 | 56548 | 3.26562 [symbol] |
| 226215_s_at | CIT | Citron (rho-interacting, serine/threonine kinase 21) | 11113 | 1.65862 [symbol] |
| 211358_s_at | CIZ1 | CDKN1A interacting zinc finger protein 1 | 25792 | 1.63870 [symbol] |
| 204662_at | CP110 | CP110 protein | 9738 | 2.40695 [symbol] |
| 209674_at | CRY1 | Cryptochrome 1 (photolyase-like) | 1407 | 2.55964 [symbol] |
| 39966_at | CSPG5 | Chondroitin sulfate proteoglycan 5 (neuroglycan C) | 10675 | 3.71092 [symbol] |
| 218898_at | CT120 | Family with sequence similarity 57, member A | 79850 | 1.93705 [symbol] |
| 204190_at | D13S106E | Chromosome 13 open reading frame 22 | 10208 | 0.691601 |
| 209570_s_at | D4S234E | DNA segment on chromosome 4 (unique) 234 expressed sequence | 27065 | 1.58660 [symbol] |
| 203302_at | DCK | Deoxycytidine kinase | 1633 | 2.83670 [symbol] |
| 222889_at | DCLRE1B | DNA cross-link repair 1B (PSO2 homolog, S. cerevisiae) | 64858 | 3.10686 [symbol] |
| 209094_at | DDAH1 | Dimethylarginine dimethylaminohydrolase 1 | 23576 | 2.62912 [symbol] |
| 226986_at | DKFZP434J154 | WIPI49-like protein 2 | 26100 | 1.54437 [symbol] |
| 204382_at | DKFZP564C103 | Embryo brain specific protein | 26151 | 0.62182 [symbol] |
| 212730_at | DMN | Data not found | 23336 | 7.18846 [symbol] |
| 213088_s_at | DNAJC9 | DnaJ (Hsp40) homolog, subfamily C, member 9 | 23234 | 1.67666 [symbol] |
| 221677_s_at | DONSON | Downstream neighbor of SON | 29980 | 1.67535 [symbol] |
| 207267_s_at | DSCR6 | Down syndrome critical region gene 6 | 53820 | 2.86780 [symbol] |
| 201908_at | DVL3 | Dishevelled, dsh homolog 3 (Drosophila) | 1857 | 1.51530 [symbol] |
| 228033_at | E2F7 | E2F transcription factor 7 | 144455 | 4.06866 [symbol] |
| 204540_at | EEF1A2 | Eukaryotic translation elongation factor 1 alpha 2 | 1917 | 2.573621 |
| 214805_at | EIF4A1 | Eukaryotic translation initiation factor 4A, isoform 1 | 1973 | 0.64096 [symbol] |
| 201313_at | ENO2 | Enolase 2 (gamma, neuronal) | 2026 | 21.1196 [symbol] |
| 219731_at | ENTPD1 | Ectonucleoside triphosphate diphosphohydrolase 1 | 953 | 1.499271 |
| 227386_s_at | EPB41 | Data not found | 2035 | 2.07895 [symbol] |
| 220161_s_at | EPB41L4B | Erythrocyte membrane protein band 4.1 like 4B | 54566 | 1.49469 [symbol] |
| 203499_at | EPHA2 | EPH receptor A2 | 1969 | 0.53331 [symbol] |
| 203358_s_at | EZH2 | Enhancer of zeste homolog 2 (Drosophila) | 2146 | 1.750031 |
| 203806_s_at | FANCA | Fanconi anemia, complementation group A | 2175 | 3.017421 |
| 203805_s_at | FANCA | Fanconi anemia, complementation group A | 2175 | 2.138861 |
| 212231_at | FBXO21 | F-box protein 21 | 23014 | 1.68698 [symbol] |
| 204768_s_at | FEN1 | Flap structure-specific endonuclease 1 | 2237 | 2.102911 |
| 204767_s_at | FEN1 | Flap structure-specific endonuclease 1 | 2237 | 3.98381 [symbol] |
| 206404_at | FGF9 | Fibroblast growth factor 9 (glia-activating factor) | 2254 | 4.42812 [symbol] |
| 204379_s_at | FGFR3 | Fibroblast growth factor receptor 3 (achondroplasia, thanatophoric dwarfism) | 2261 | 4.22937 [symbol] |
| 218974_at | FLJ10159 | Hypothetical protein FLJ10159 | 55084 | 3.34923 [symbol] |
| 219760_at | FLJ10490 | Hypothetical protein FLJ10490 | 55150 | 2.73325 [symbol] |
| 228774_at | FLJ12643 | Chromosome 9 open reading frame 81 | 84131 | 1.61189 [symbol] |
| 204365_s_at | FLJ13110 | Chromosome 2 open reading frame 23 | 65055 | 1.951871 |
| 204364_s_at | FLJ13110 | Chromosome 2 open reading frame 23 | 65055 | 3.98011 [symbol] |
| 222760_at | FLJ14299 | Hypothetical protein FLJ14299 | 80139 | 3.41043 [symbol] |
| 226487_at | FLJ14721 | Hypothetical protein FLJ14721 | 84915 | 4.00535 [symbol] |
| 223171_at | FLJ20071 | Dymeclin | 54808 | 1.509261 |
| 218510_x_at | FLJ20152 | Hypothetical protein FLJ20152 | 54463 | 1.63454 [symbol] |
| 217899_at | FLJ20254 | Hypothetical protein FLJ20254 | 54867 | 1.55549 [symbol] |
| 225139_at | FLJ21918 | Hypothetical protein FLJ21918 | 80004 | 1.63664 [symbol] |
| 226925_at | FLJ23751 | Acid phosphatase-like 2 | 92370 | 1.75603 [symbol] |
| 230137_at | FLJ30834 | Hypothetical protein FLJ30834 | 132332 | 11.3421 [symbol] |
| 226132_s_at | FLJ31434 | mannosidase, endo-alpha-like | 149175 | 2.97665 [symbol] |
| 235144_at | FLJ31614 | RAS and EF hand domain containing | 158158 | 3.44180 [symbol] |
| 1553986_at | FLJ31614 | RAS and EF hand domain containing | 158158 | 2.05264 [symbol] |
| 236219_at | FLJ33990 | Transmembrane protein 20 | 159371 | 4.67933 [symbol] |
| 244297_at | FLJ35740 | Data not found | 253650 | 2.328871 |
| 233592_at | FLJ35740 | Data not found | 253650 | 1.91114 [symbol] |
| 240161_s_at | FLJ37927 | CDC20-like protein | 166979 | 5.22880 [symbol] |
| 227475_at | FOXQ1 | Forkhead box Q1 | 94234 | 1.44192 [symbol] |
| 219889_at | FRAT1 | Frequently rearranged in advanced T-cell lymphomas | 10023 | 1.44305 [symbol] |
| 226348_at | FUT11 | Data not found | 170384 | 1.93981 [symbol] |
| 204452_s_at | FZD1 | Frizzled homolog 1 (Drosophila) | 8321 | 2.13529 [symbol] |
| 204451_at | FZD1 | Frizzled homolog 1 (Drosophila) | 8321 | 2.01565 [symbol] |
| 204224_s_at | GCH1 | GTP cyclohydrolase 1 (dopa-responsive dystonia) | 2643 | 3.89669 [symbol] |
| 234192_s_at | GKAP42 | G kinase anchoring protein 1 | 80318 | 4.61081 [symbol] |
| 229312_s_at | GKAP42 | G kinase anchoring protein 1 | 80318 | 2.38096 [symbol] |
| 205280_at | GLRB | Glycine receptor, beta | 2743 | 2.55671 [symbol] |
| 206355_at | GNAL | Guanine nucleotide binding protein (G protein), alpha activating | 2774 | 1.40581 [symbol] |
| | | activity polypeptide, olfactory type | | |
| 214157_at | GNAS | GNAS complex locus | 2778 | 2.81958 [symbol] |
| 227769_at | GPR27 | G protein-coupled receptor 27 | 2850 | 4.10784 [symbol] |
| 242517_at | GPR54 | G protein-coupled receptor 54 | 84634 | 4.89522 [symbol] |
| 227471_at | HACE1 | HECT domain and ankyrin repeat containing, E3 ubiquitin protein ligase 1 | 57531 | 1.87602 [symbol] |
| 218603_at | HECA | Headcase homolog (Drosophila) | 51696 | 1.65309 [symbol] |
| 242890_at | HELLS | Helicase, lymphoid-specific | 3070 | 1.53036 [symbol] |
| 44783_s_at | HEY1 | Hairy/enhancer-of-split related with YRPW motif 1 | 23462 | 2.94757 [symbol] |
| 218839_at | HEY1 | Hairy/enhancer-of-split related with YRPW motif 1 | 23462 | 10.8354 [symbol] |
| 222996_s_at | HSPC195 | CXXC finger 5 | 51523 | 1.46609 [symbol] |
| 205449_at | HSU79266 | SAC3 domain containing 1 | 29901 | 3.19477 [symbol] |
| 224361_s_at | IL17RB | Interleukin 17 receptor B | 55540 | 4.99100 [symbol] |
| 224156_x_at | IL17RB | Interleukin 17 receptor B | 55540 | 2.97575 [symbol] |
| 219255_x_at | IL17RB | Interleukin 17 receptor B | 55540 | 3.68079 [symbol] |
| 205067_at | IL1B | Interleukin 1, beta | 3553 | 0.65147 [symbol] |
| 205258_at | INHBB | Inhibin, beta B (activin AB beta polypeptide) | 3625 | 2.56835 [symbol] |
| 227432_s_at | INSR | Insulin receptor | 3643 | 2.01272 [symbol] |
| 226216_at | INSR | Insulin receptor | 3643 | 2.027351 |
| 229139_at | JPH1 | Junctophilin 1 | 56704 | 2.30127 [symbol] |
| 222668_at | KCTD15 | Potassium channel tetramerisation domain containing 15 | 79047 | 1.47786 [symbol] |
| 222664_at | KCTD15 | Potassium channel tetramerisation domain containing 15 | 79047 | 1.59439 [symbol] |
| 238077_at | KCTD6 | Potassium channel tetramerisation domain containing 6 | 200845 | 2.91065 [symbol] |
| 209781_s_at | KHDRBS3 | KH domain containing, RNA binding, signal transduction associated 3 | 10656 | 2.29463 [symbol] |
| 212057_at | KIAA0182 | KIAA0182 protein | 23199 | 1.588571 |
| 212056_at | KIAA0182 | KIAA0182 protein | 23199 | 1.91479 [symbol] |
| 206102_at | KIAA0186 | DNA replication complex GINS protein PSF1 | 9837 | 2.159301 |
| 1569796_s_at | KIAA0534 | Attractin-like 1 | 26033 | 3.07113 [symbol] |
| 212492_s_at | KIAA0876 | Jumonji domain containing 2B | 23030 | 0.73908 [symbol] |
| 212792_at | KIAA0877 | KIAA0877 protein | 23333 | 1.68094 [symbol] |
| 212956_at | KIAA0882 | KIAA0882 protein | 23158 | 2.14381 [symbol] |
| 228051_at | KIAA1244 | KIAA1244 | 57221 | 2.72262 [symbol] |
| 218829_s_at | KIAA1416 | Chromodomain helicase DNA binding protein 7 | 55636 | 1.46432 [symbol] |
| 218418_s_at | KIAA1518 | Ankyrin repeat domain 25 | 25959 | 1.45179 [symbol] |
| 231851_at | KIAA1579 | Hypothetical protein FLJ10770 | 55225 | 2.03851 [symbol] |
| 228565_at | KIAA1804 | Mixed lineage kinase 4 | 84451 | 2.12404 [symbol] |
| 226796_at | LOC116236 | Hypothetical protein LOC116236 | 116236 | 6.47382 [symbol] |
| 227804_at | LOC116238 | Data not found | 116238 | 2.02645 [symbol] |
| 229582_at | LOC125476 | Chromosome 18 open reading frame 37 | 125476 | 0.61506 [symbol] |
| 226702_at | LOC129607 | Hypothetical protein LOC129607 | 129607 | 4.67036 [symbol] |
| 235391_at | LOC137392 | Similar to CG6405 gene product | 137392 | 2.63126 [symbol] |
| 235177_at | LOC151194 | Similar to hepatocellular carcinoma-associated antigen HCA557b | 151194 | 2.447971 |
| 212771_at | LOC221061 | Chromosome 10 open reading frame 38 | 221061 | 1.33716 [symbol] |
| 221823_at | LOC90355 | Hypothetical gene supported by AF038182; BC009203 | 90355 | 1.35365 [symbol] |
| 225650_at | LOC90378 | Sterile alpha motif domain containing 1 | 90378 | 2.29697 [symbol] |
| 211596_s_at | LRIG1 | Leucine-rich repeats and immunoglobulin-like domains 1 | 26018 | 1.47019 [symbol] |
| 212850_s_at | LRP4 | Low density lipoprotein receptor-related protein 4 | 4038 | 2.08177 [symbol] |
| 212282_at | MAC30 | Hypothetical protein MAC30 | 27346 | 2.44231 [symbol] |
| 212281_s_at | MAC30 | Hypothetical protein MAC30 | 27346 | 2.75857 [symbol] |
| 212279_at | MAC30 | Hypothetical protein MAC30 | 27346 | 2.09292 [symbol] |
| 207069_s_at | MADH6 | SMAD, mothers against DPP homolog 6 (Drosophila) | 4091 | 12.0471 [symbol] |
| 225478_at | MFHAS1 | Malignant fibrous histiocytoma amplified sequence 1 | 9258 | 1.52171 [symbol] |
| 218358_at | MGC11256 | Hypothetical protein MGC11256 | 79174 | 2.005251 |
| 233480_at | MGC3222 | Transmembrane protein 43 | 79188 | 0.66360 [symbol] |
| 226912_at | MGC42530 | Zinc finger, DHHC domain containing 23 | 254887 | 5.82483 [symbol] |
| 235005_at | MGC4562 | Hypothetical protein MGC4562 | 115752 | 1.75975 [symbol] |
| 226605_at | MGC4618 | Hypothetical protein MGC4618 | 84286 | 0.71452 [symbol] |
| 227764_at | MGC52057 | Hypothetical protein MGC52057 | 130574 | 4.56982 [symbol] |
| 222728_s_at | MGC5306 | Hypothetical protein MGC5306 | 79101 | 0.51188 [symbol] |
| 218750_at | MGC5306 | Hypothetical protein MGC5306 | 79101 | 0.60629 [symbol] |
| 201764_at | MGC5576 | Hypothetical protein MGC5576 | 79022 | 3.00888 [symbol] |
| 203365_s_at | MMP15 | Matrix metalloproteinase 15 (membrane-inserted) | 4324 | 15.4442 [symbol] |
| 225185_at | MRAS | Muscle RAS oncogene homolog | 22808 | 1.77734 [symbol] |
| 204798_at | MYB | V-myb myeloblastosis viral oncogene homolog (avian) | 4602 | 7.59093 [symbol] |
| 201970_s_at | NASP | Nuclear autoantigenic sperm protein (histone-binding) | 4678 | 1.94957 [symbol] |
| 221805_at | NEFL | Neurofilament, light polypeptide 68 kDa | 4747 | 4.78639 [symbol] |
| 222774_s_at | NETO2 | Neuropilin (NRP) and tolloid (TLL)-like 2 | 81831 | 1.80459 [symbol] |
| 218888_s_at | NETO2 | Neuropilin (NRP) and tolloid (TLL)-like 2 | 81831 | 2.35614 [symbol] |
| 225921_at | NIN | Ninein (GSK3B interacting protein) | 51199 | 1.65934 [symbol] |
| 209505_at | NR2F1 | Nuclear receptor subfamily 2, group F, member 1 | 7025 | 5.15546 [symbol] |
| 206550_s_at | NUP155 | Nucleoporin 155 kDa | 9631 | 1.958611 |
| 227379_at | OACT1 | O-acyltransferase (membrane bound) domain containing 1 | 154141 | 2.02574 [symbol] |
| 226350_at | OPN3 | Opsin 3 (encephalopsin, panopsin) | 23596 | 2.50768 [symbol] |
| 230104_s_at | p25 | Brain-specific protein p25 alpha | 11076 | 4.12758 [symbol] |
| 201202_at | PCNA | Proliferating cell nuclear antigen | 5111 | 2.67315 [symbol] |
| 219295_s_at | PCOLCE2 | Procollagen C-endopeptidase enhancer 2 | 26577 | 2.07351 [symbol] |
| 212522_at | PDE8A | Phosphodiesterase 8A | 5151 | 1.61352 [symbol] |
| 212094_at | PEG10 | Paternally expressed 10 | 23089 | 5.58443 [symbol] |
| 212092_at | PEG10 | Paternally expressed 10 | 23089 | 3.97661 [symbol] |
| 244677_at | PER1 | Period homolog 1 (Drosophila) | 5187 | 0.584531 |
| 202464_s_at | PFKFB3 | 6-phosphofructo-2-kinase/fructose-2,6-biphosphatase 3 | 5209 | 1.90144 [symbol] |
| 225048_at | PHF10 | PHD finger protein 10 | 55274 | 1.89300 [symbol] |
| 219126_at | PHF10 | PHD finger protein 10 | 55274 | 2.06868 [symbol] |
| 212726_at | PHF2 | PHD finger protein 2 | 5253 | 1.98426 [symbol] |
| 209780_at | PHTF2 | Putative homeodomain transcription factor 2 | 57157 | 2.02395 [symbol] |
| 202927_at | PIN1 | Protein (peptidyl-prolyl cis/trans isomerase) NIMA-interacting 1 | 5300 | 2.69936 [symbol] |
| 226299_at | pknbeta | Protein kinase N3 | 29941 | 2.63567 [symbol] |
| 216218_s_at | PLCL2 | Phospholipase C-like 2 | 23228 | 7.25059 [symbol] |
| 38671_at | PLXND1 | Plexin D1 | 23129 | 2.43959 [symbol] |
| 216026_s_at | POLE | Polymerase (DNA directed), epsilon | 5426 | 2.33608 [symbol] |
| 205909_at | POLE2 | Polymerase (DNA directed), epsilon 2 (p59 subunit) | 5427 | 2.18806 [symbol] |
| 212230_at | PPAP2B | Phosphatidic acid phosphatase type 2B | 8613 | 2.36371 [symbol] |
| 235266_at | PRO2000 | ATPase family, AAA domain containing 2 | 29028 | 2.34516 [symbol] |
| 228401_at | PRO2000 | ATPase family, AAA domain containing 2 | 29028 | 2.56315 [symbol] |
| 222740_at | PRO2000 | ATPase family, AAA domain containing 2 | 29028 | 2.25207 [symbol] |
| 218782_s_at | PRO2000 | ATPase family, AAA domain containing 2 | 29028 | 2.08585 [symbol] |
| 209337_at | PSIP2 | PC4 and SFRS1 interacting protein 1 | 11168 | 1.82594 [symbol] |
| 205128_x_at | PTGS1 | Prostaglandin-endperoxide synthase 1 (prostaglandin G/H synthase and cyclooxygenase) | 5742 | 0.65632 [symbol] |
| 201606_s_at | PWP1 | Nuclear phosphoprotein similar to S. cerevisiae PWP1 | 11137 | 0.73897 [symbol] |
| 219076_s_at | PXMP2 | Peroxisomal membrane protein 2, 22 kDa | 5827 | 3.30950 [symbol] |
| 50965_at | RAB26 | RAB26, member RAS oncogene family | 25837 | 2.16868 [symbol] |
| 219562_at | RAB26 | RAB26, member RAS oncogene family | 25837 | 2.75862 [symbol] |
| 218585_s_at | RAMP | RA-regulated nuclear matrix-associated protein | 51514 | 2.41875 [symbol] |
| 1553015_a_at | RECQL4 | RecQ protein-like 4 | 9401 | 2.74856 [symbol] |
| 213338_at | RIS1 | Ras-induced senescence 1 | 25907 | 5.37168 [symbol] |
| 212027_at | RNPC7 | RNA binding motif protein 25 | 58517 | 0.629131 |
| 201529_s_at | RPA1 | Replication protein A1, 70 kDa | 6117 | 1.666561 |
| 214291_at | RPL17 | Data not found | 6139 | 0.80180 [symbol] |
| 238156_at | RPS6 | Ribosomal protein S6 | 6194 | 0.52423 [symbol] |
| 221523_s_at | RRAGD | Ras-related GTP binding D | 58528 | 6.25606 [symbol] |
| 228550_at | RTN4R | Reticulon 4 receptor | 65078 | 2.332371 |
| 204198_s_at | RUNX3 | Runt-related transcription factor 3 | 864 | 1.41010 [symbol] |
| 204197_s_at | RUNX3 | Runt-related transcription factor 3 | 864 | 1.539241 |
| 207049_at | SCN8A | Sodium channel, voltage gated, type VIII, alpha | 6334 | 5.477041 |
| 203453_at | SCNN1A | Sodium channel, nonvoltage-gated 1 alpha | 6337 | 0.59889 [symbol] |
| 1569594_a_at | SDCCAG1 | Serologically defined colon cancer antigen 1 | 9147 | 0.671431 |
| 223283_s_at | SDCCAG33 | Serologically defined colon cancer antigen 33 | 10194 | 2.43012 [symbol] |
| 223282_at | SDCCAG33 | Serologically defined colon cancer antigen 33 | 10194 | 2.93894 [symbol] |
| 213370_s_at | SFMBT1 | Scm-like with four mbt domains 1 | 51460 | 1.76612 [symbol] |
| 206108_s_at | SFRS6 | Splicing factor, arginine/serine-rich 6 | 6431 | 0.53886 [symbol] |
| 213649_at | SFRS7 | Splicing factor, arginine/serine-rich 7, 35 kDa | 6432 | 0.62728 [symbol] |
| 204979_s_at | SH3BGR | SH3 domain binding glutamic acid-rich protein | 6450 | 2.28187 [symbol] |
| 227923_at | SHANK3 | SH3 and multiple ankyrin repeat domains 3 | 85358 | 3.20482 [symbol] |
| 39705_at | SIN3B | SIN3 homolog B, transcription regulator (yeast) | 23309 | 0.733201 |
| 229009_at | SIX5 | Sine oculis homeobox homolog 5 (Drosophila) | 147912 | 2.17323 [symbol] |
| 230748_at | SLC16A6 | Solute carrier family 16 (monocarboxylic acid transporters), member 6 | 9120 | 1.964451 |
| 203340_s_at | SLC25A12 | Solute carrier family 25 (mitochondrial carrier, Aralar), member 12 | 8604 | 1.49561 [symbol] |
| 203339_at | SLC25A12 | Solute carrier family 25 (mitochondrial carrier, Aralar), member 12 | 8604 | 2.09052 [symbol] |
| 222217_s_at | SLC27A3 | Solute carrier family 27 (fatty acid transporter), member 3 | 11000 | 3.22102 [symbol] |
| 201349_at | SLC9A3R1 | Solute carrier family 9 (sodium/hydrogen exchanger), isoform 3 regulator 1 | 9368 | 1.93212 [symbol] |
| 204432_at | SOX12 | SRY (sex determining region Y)-box 12 | 6666 | 1.45560 [symbol] |
| 225752_at | SPG6 | Non imprinted in Prader-Willi/Angelman syndrome 1 | 123606 | 1.754731 |
| 202308_at | SREBF1 | Data not found | 6720 | 0.64121 [symbol] |
| 203016_s_at | SSX2IP | Synovial sarcoma, X breakpoint 2 interacting protein | 117178 | 1.22815 [symbol] |
| 209478_at | STRA13 | Stimulated by retinoic acid 13 homolog (mouse) | 201254 | 4.59235 [symbol] |
| 202260_s_at | STXBP1 | Syntaxin binding protein 1 | 6812 | 1.90707 [symbol] |
| 213090_s_at | TAF4 | TAF4 RNA polymerase II, TATA box binding protein (TBP)-associated factor, 135 kDa | 6874 | 1.965851 |
| 41037_at | TEAD4 | TEA domain family member 4 | 7004 | 1.82034 [symbol] |
| 212330_at | TFDP1 | Transcription factor Dp-1 | 7027 | 1.41689 [symbol] |
| 213135_at | TIAM1 | T-cell lymphoma invasion and metastasis 1 | 7074 | 2.31210 [symbol] |
| 228256_s_at | TIGA1 | TIGA1 | 114915 | 2.10320 [symbol] |
| 225388_at | TM4SF9 | Tetraspanin 5 | 10098 | 1.85574 [symbol] |
| 225387_at | TM4SF9 | Tetraspanin 5 | 10098 | 2.46785 [symbol] |
| 219892_at | TM6SF1 | Transmembrane 6 superfamily member 1 | 53346 | 5.61423 [symbol] |
| 204137_at | TM7SF1 | Transmembrane 7 superfamily member 1 (upregulated in kidney) | 7107 | 2.21579 [symbol] |
| 207291_at | TMG4 | Proline rich Gla (G-carboxyglutamic acid) 4 (transmembrane) | 79056 | 2.56675 [symbol] |
| 226186_at | TMOD2 | Tropomodulin 2 (neuronal) | 29767 | 3.53330 [symbol] |
| 216005_at | TNC | Tenascin C (hexabrachion) | 3371 | 0.50123 [symbol] |
| 202644_s_at | TNFAIP3 | Tumor necrosis factor, alpha-induced protein 3 | 7128 | 0.533461 |
| 213885_at | TRIM3 | Tripartite motif-containing 3 | 10612 | 1.66401 [symbol] |
| 239694_at | TRIM7 | Tripartite motif-containing 7 | 81786 | 1.88929 [symbol] |
| 228956_at | UGT8 | UDP glycosyltransferase 8 (UDP-galactose ceramide galactosyltransferase) | 7368 | 3.68682 [symbol] |
| 208358_s_at | UGT8 | UDP glycosyltransferase 8 (UDP-galactose ceramide galactosyltransferase) | 7368 | 2.396441 |
| 210021_s_at | UNG2 | Uracil-DNA glycosylase 2 | 10309 | 2.69495 [symbol] |
| 231227_at | WNT5A | Wingless-type MMTV integration site family, member 5A | 7474 | 2.199931 |
| 213425_at | WNT5A | Wingless-type MMTV integration site family, member 5A | 7474 | 2.32192 [symbol] |
| 205990_s_at | WNT5A | Wingless-type MMTV integration site family, member 5A | 7474 | 1.76742 [symbol] |
| 203712_at | XTP5 | KIAA0020 | 9933 | 0.70414 [symbol] |
| 204234_s_at | ZNF195 | Zinc finger protein 195 | 7748 | 0.68930 [symbol] |
| 222227_at | ZNF236 | Zinc finger protein 236 | 7776 | 0.24313 [symbol] |
| 225382_at | ZNF275 | Zinc finger protein 275 | 10838 | 2.30665 [symbol] |
| 229551_x_at | ZNF367 | Zinc finger protein 367 | 195828 | 4.68695 [symbol] |
| 204026_s_at | ZWINT | Data not found | 11130 | 1.50004 [symbol] |
| 59697_at | — | Data not found | — | 1.44507 [symbol] |
| 244467_at | — | Data not found | — | 2.86596 [symbol] |
| 241957_x_at | — | Data not found | — | 2.256321 |
| 241464_s_at | — | Data not found | — | 0.63837 [symbol] |
| 238513_at | — | Data not found | — | 2.37249 [symbol] |
| 237187_at | — | Data not found | — | 2.10057 [symbol] |
| 236488_s_at | — | Data not found | — | 1.90155 [symbol] |
| 236289_at | — | Data not found | — | 2.21540 [symbol] |
| 235919_at | — | Data not found | — | 2.37030 [symbol] |
| 233364_s_at | — | Data not found | — | 0.37494 [symbol] |
| 229899_s_at | — | Data not found | 375100 | 0.58273 [symbol] |
| 229715_at | — | Data not found | — | 1.86765 [symbol] |
| 229691_at | — | Data not found | 376285 | 3.54739 [symbol] |
| 229656_s_at | — | Data not found | 344403 | 4.62163 [symbol] |
| 228955_at | — | Data not found | — | 2.30280 [symbol] |
| 228238_at | — | Data not found | — | 0.49783 [symbol] |
| 228180_at | — | Data not found | — | 0.588831 |
| 227193_at | — | Data not found | — | 3.73810 [symbol] |
| 226618_at | — | Similar to CG4502-PA | 134111 | 8.32345 [symbol] |
| 226549_at | — | Data not found | — | 11.7343 [symbol] |
| 226548_at | — | Data not found | Hs.97837 | 30.4793 [symbol] |
| 225716_at | — | Data not found | — | 2.80510 [symbol] |
| 225467_s_at | — | Data not found | — | 0.748061 |
| 216843_x_at | — | Data not found | — | 0.77992 [symbol] |
| 212693_at | — | Data not found | — | 0.93525 [symbol] |
| 209815_at | — | Data not found | — | 3.16762 [symbol] |
| 1568597_at | — | Data not found | — | 2.123801 |
| 1568408_x_at | — | Data not found | — | 0.58864 [symbol] |
| 1556486_at | — | Data not found | — | 2.91700 [symbol] |
| 1554007_at | — | Data not found | — | 4.80020 [symbol] |
| Ras | | | | |
| 203504_s_at | ABCA1 | ATP-binding cassette, sub-family A (ABC1), member 1 | 19 | 0.33115 [symbol] |
| 205179_s_at | ADAM8 | A disintegrin and metalloproteinase domain 8 | 101 | 5.65848 [symbol] |
| 205180_s_at | ADAM8 | A disintegrin and metalloproteinase domain 8 | 101 | 3.84752 [symbol] |
| 219935_at | ADAMTS5 | A disintegrin-like and metalloprotease (reprolysin type) with | 11096 | 0.20599 [symbol] |
| | | thrombospondin type 1 motif, 5 (aggrecanase-2) | | |
| 206170_at | ADRB2 | Adrenergic, beta-2-, receptor, surface | 154 | 3.48743 [symbol] |
| 231067_s_at | AKAP12 | A kinase (PRKA) anchor protein (gravin) 12 | 9590 | 5.03982 [symbol] |
| 223333_s_at | ANGPTL4 | Angiopoietin-like 4 | 51129 | 10.8642 [symbol] |
| 221009_s_at | ANGPTL4 | Angiopoietin-like 4 | 51129 | 6.60934 [symbol] |
| 203946_s_at | ARG2 | Arginase, type II | 384 | 3.40236 [symbol] |
| 203263_s_at | ARHGEF9 | Cdc42 guanine nucleotide exchange factor (GEF) 9 | 23229 | 0.32279 [symbol] |
| 220658_s_at | ARNTL2 | Aryl hydrocarbon receptor nuclear translocator-like 2 | 56938 | 1.74633 [symbol] |
| 209281_s_at | ATP2B1 | ATPase, Ca++ transporting, plasma membrane 1 | 490 | 3.67994 [symbol] |
| 212930_at | ATP2B1 | ATPase, Ca++ transporting, plasma membrane 1 | 490 | 3.47287 [symbol] |
| 225612_s_at | B3GNT5 | UDP-GlcNAc:betaGal beta-1,3-N-acetylglucosaminyltransferase 5 | 84002 | 5.62373 [symbol] |
| 1554835_a_at | B3GNT5 | UDP-GlcNAc:betaGal beta-1,3-N-acetylglucosaminyltransferase 5 | 84002 | 5.37789 [symbol] |
| 228498_at | B4GALT1 | UDP-Gal:betaGlcNAc beta 1,4-galactosyltransferase, polypeptide 1 | 2683 | 3.201531 |
| 208002_s_at | BACH | Brain acyl-CoA hydrolase | 11332 | 2.18061 [symbol] |
| 203140_at | BCL6 | B-cell CLL/lymphoma 6 (zinc finger protein 51) | 604 | 0.28988 [symbol] |
| 209373_at | BENE | BENE protein | 7851 | 2.85152 [symbol] |
| 205289_at | BMP2 | Bone morphogenetic protein 2 | 650 | 14.6418 [symbol] |
| 205290_s_at | BMP2 | Bone morphogenetic protein 2 | 650 | 22.1539 [symbol] |
| 219563_at | C14orf139 | Chromosome 14 open reading frame 139 | 79686 | 5.02996 [symbol] |
| 1558378_a_at | C14orf78 | Chromosome 14 open reading frame 78 | 113146 | 0.28177 [symbol] |
| 60474_at | C20orf42 | Chromosome 20 open reading frame 42 | 55612 | 7.93008 [symbol] |
| 218796_at | C20orf42 | Chromosome 20 open reading frame 42 | 55612 | 11.7762 [symbol] |
| 229545_at | C20orf42 | Chromosome 20 open reading frame 42 | 55612 | 7.06025 [symbol] |
| 1552575_a_at | C6orf141 | Chromosome 6 open reading frame 141 | 135398 | 3.32148 [symbol] |
| 202241_at | C8FW | Tribbles homolog 1 (Drosophila) | 10221 | 3.95011 [symbol] |
| 207243_s_at | CALM2 | Calmodulin 2 (phosphorylase kinase, delta) | 805 | 2.65181 [symbol] |
| 214845_s_at | CALU | Calumenin | 813 | 3.082181 |
| 200756_x_at | CALU | Calumenin | 813 | 2.32567 [symbol] |
| 227364_at | CAPZA1 | Capping protein (actin filament) muscle Z-line, alpha 1 | 829 | 3.45260 [symbol] |
| 206011_at | CASP1 | Caspase 1, apoptosis-related cysteine protease (interleukin 1, beta, convertase) | 834 | 0.41028 [symbol] |
| 226032_at | CASP2 | Caspase 2, apoptosis-related cysteine protease (neural precursor | 835 | 0.52737 [symbol] |
| | | cell expressed, developmentally do [symbol] | | |
| 205476_at | CCL20 | Chemokine (C-C motif) ligand 20 | 6364 | 61.8252 [symbol] |
| 205899_at | CCNA1 | Cyclin A1 | 8900 | 3.95434 [symbol] |
| 241495_at | CCNL1 | Cyclin L1 | 57018 | 0.23736 [symbol] |
| 218451_at | CDCP1 | CUB domain containing protein 1 | 64866 | 4.16130 [symbol] |
| 226372_at | CHST11 | Carbohydrate (chondroitin 4) sulfotransferase 11 | 50515 | 4.01326 [symbol] |
| 219500_at | CLC | Cardiotrophin-like cytokine factor 1 | 23529 | 5.20740 [symbol] |
| 230603_at | COL27A1 | Collagen, type XXVII, alpha 1 | 85301 | 0.209111 |
| 208960_s_at | COPEB | Kruppel-like factor 6 | 1316 | 3.14278 [symbol] |
| 208961_s_at | COPEB | Kruppel-like factor 6 | 1316 | 3.82494 [symbol] |
| 207945_s_at | CSNK1D | Casein kinase 1, delta | 1453 | 1.98115 [symbol] |
| 225756_at | CSNK1E | Casein kinase 1, epsilon | 1454 | 3.41026 [symbol] |
| 202332_at | CSNK1E | Casein kinase 1, epsilon | 1454 | 2.50858 [symbol] |
| 222265_at | CTEN | C-terminal tensin-like | 84951 | 2.94986 [symbol] |
| 204470_at | CXCL1 | Chemokine (C—X—C motif) ligand 1 (melanoma growth stimulating activity, alpha) | 2919 | 5.61959 [symbol] |
| 209774_x_at | CXCL2 | Chemokine (C—X—C motif) ligand 2 | 2920 | 8.73050 [symbol] |
| 207850_at | CXCL3 | Chemokine (C—X—C motif) ligand 3 | 2921 | 29.8426 [symbol] |
| 215101_s_at | CXCL5 | Chemokine (C—X—C motif) ligand 5 | 6374 | 6.95267 [symbol] |
| 202436_s_at | CYP1B1 | Cytochrome P450, family 1, subfamily B, polypeptide 1 | 1545 | 0.32866 [symbol] |
| 202435_s_at | CYP1B1 | Cytochrome P450, family 1, subfamily B, polypeptide 1 | 1545 | 0.20113 [symbol] |
| 205676_at | CYP27B1 | Cytochrome P450, family 27, subfamily B, polypeptide 1 | 1594 | 3.19969 [symbol] |
| 227109_at | CYP2R1 | Cytochrome P450, family 2, subfamily R, polypeptide 1 | 120227 | 0.34285 [symbol] |
| 201925_s_at | DAF | Decay accelerating factor for complement (CD55, Cromer blood group system) | 1604 | 7.26920 [symbol] |
| 201926_s_at | DAF | Decay accelerating factor for complement (CD55, Cromer blood group system) | 1604 | 4.86208 [symbol] |
| 1555950_a_at | DAF | Decay accelerating factor for complement (CD55, Cromer blood group system) | 1604 | 4.350231 |
| 208151_x_at | DDX17 | DEAD (Asp-Glu-Ala-Asp) box polypeptide 17 | 10521 | 0.21528 [symbol] |
| 208719_s_at | DDX17 | DEAD (Asp-Glu-Ala-Asp) box polypeptide 17 | 10521 | 0.19194 [symbol] |
| 204420_at | DIPA | Hepatitis delta antigen-interacting protein A | 11007 | 9.95404 [symbol] |
| 235263_at | DKFZP434A0131 | DKFZp434A0131 protein | 54441 | 0.46624 [symbol] |
| 224215_s_at | DLL1 | Delta-like 1 (Drosophila) | 28514 | 0.27797 [symbol] |
| 215210_s_at | DLST | Dihydrolipoamide S-succinyltransferase (E2 component of 2-oxo-glutarate complex) | 1743 | 2.504691 |
| 204720_s_at | DNAJC6 | DnaJ (Hsp40) homolog, subfamily C, member 6 | 9829 | 0.30782 [symbol] |
| 38037_at | DTR | Heparin-binding EGF-like growth factor | 1839 | 20.8149 [symbol] |
| 203821_at | DTR | Heparin-binding EGF-like growth factor | 1839 | 17.0206 [symbol] |
| 201041_s_at | DUSP1 | Dual specificity phosphatase 1 | 1843 | 21.2932 [symbol] |
| 201044_x_at | DUSP1 | Dual specificity phosphatase 1 | 1843 | 45.4933 [symbol] |
| 204014_at | DUSP4 | Dual specificity phosphatase 4 | 1846 | 4.90201 [symbol] |
| 204015_s_at | DUSP4 | Dual specificity phosphatase 4 | 1846 | 3.14847 [symbol] |
| 209457_at | DUSP5 | Dual specificity phosphatase 5 | 1847 | 7.53307 [symbol] |
| 208891_at | DUSP6 | Dual specificity phosphatase 6 | 1848 | 7.62005 [symbol] |
| 208893_s_at | DUSP6 | Dual specificity phosphatase 6 | 1848 | 8.64368 [symbol] |
| 208892_s_at | DUSP6 | Dual specificity phosphatase 6 | 1848 | 5.35213 [symbol] |
| 206722_s_at | EDG4 | Endothelial differentiation, lysophosphatidic acid G-protein-coupled receptor, 4 | 9170 | 2.28486 [symbol] |
| 202711_at | EFNB1 | Ephrin-B1 | 1947 | 3.50637 [symbol] |
| 227404_s_at | EGR1 | Early growth response 1 | 1958 | 5.17121 [symbol] |
| 201694_s_at | EGR1 | Early growth response 1 | 1958 | 3.14462 [symbol] |
| 209039_x_at | EHD1 | EH-domain containing 1 | 10938 | 2.57190 [symbol] |
| 221773_at | ELK3 | ELK3, ETS-domain protein (SRF accessory protein 2) | 2004 | 4.25693 [symbol] |
| 203499_at | EPHA2 | EPH receptor A2 | 1969 | 7.32631 [symbol] |
| 205767_at | EREG | Epiregulin | 2069 | 13.6492 [symbol] |
| 202081_at | ETR101 | Immediate early response 2 | 9592 | 4.26699 [symbol] |
| 210638_s_at | FBXO9 | F-box protein 9 | 26268 | 0.44994 [symbol] |
| 203639_s_at | FGFR2 | Fibroblast growth factor receptor 2 (bacteria-expressed kinase, | 2263 | 0.29501 [symbol] |
| | | keratinocyte growth factor receptor, c [symbol] | | |
| 217943_s_at | FLJ10350 | Hypothetical protein FLJ10350 | 55700 | 2.50432 [symbol] |
| 229676_at | FLJ10486 | PAP associated domain containing 1 | 55149 | 3.09041 [symbol] |
| 219235_s_at | FLJ13171 | Phosphatase and actin regulator 4 | 65979 | 0.53274 [symbol] |
| 219388_at | FLJ13782 | Transcription factor CP2-like 3 | 79977 | 0.43855 [symbol] |
| 227180_at | FLJ23563 | ELOVL family member 7, elongation of long chain fatty acids (yeast) | 79993 | 7.36711 [symbol] |
| 238063_at | FLJ32028 | Hypothetical protein FLJ32028 | 201799 | 3.59229 [symbol] |
| 235390_at | FLJ36754 | Hypothetical protein FLJ36754 | 285672 | 2.98709 [symbol] |
| 1553581_s_at | FLJ36754 | Hypothetical protein FLJ36754 | 285672 | 4.205241 |
| 230769_at | FLJ37099 | FLJ37099 protein | 163259 | 2.60332 [symbol] |
| 226908_at | FLJ90440 | Leucine-rich repeats and immunoglobulin-like domains 3 | 121227 | 0.17131 [symbol] |
| 1560017_at | FLJ90492 | SMILE protein | 160418 | 0.08943 [symbol] |
| 208614_s_at | FLNB | Filamin B, beta (actin binding protein 278) | 2317 | 2.898411 |
| 208613_s_at | FLNB | Filamin B, beta (actin binding protein 278) | 2317 | 3.07506 [symbol] |
| 219250_s_at | FLRT3 | Fibronectin leucine rich transmembrane protein 3 | 23767 | 2.18293 [symbol] |
| 214701_s_at | FN1 | Fibronectin 1 | 2335 | 0.20338 [symbol] |
| 209189_at | FOS | V-fos FBJ murine osteosarcoma viral oncogene homolog | 2353 | 158.4641 |
| 227475_at | FOXQ1 | Forkhead box Q1 | 94234 | 3.22701 [symbol] |
| 213524_s_at | G0S2 | Putative lymphocyte G0/G1 switch gene | 50486 | 8.02825 [symbol] |
| 204457_s_at | GAS1 | Growth arrest-specific 1 | 2619 | 0.03306 [symbol] |
| 215243_s_at | GJB3 | Gap junction protein, beta 3, 31 kDa (connexin 31) | 2707 | 6.217691 |
| 205490_x_at | GJB3 | Gap junction protein, beta 3, 31 kDa (connexin 31) | 2707 | 5.81269 [symbol] |
| 206156_at | GJB5 | Gap junction protein, beta 5 (connexin 31.1) | 2709 | 5.19162 [symbol] |
| 215977_x_at | GK | Glycerol kinase | 2710 | 2.96814 [symbol] |
| 225706_at | GLCCI1 | Glucocorticoid induced transcript 1 | 113263 | 0.39418 [symbol] |
| 219267_at | GLTP | Glycolipid transfer protein | 51228 | 3.68322 [symbol] |
| 226177_at | GLTP | Glycolipid transfer protein | 51228 | 3.59202 [symbol] |
| 221050_s_at | GTPBP2 | GTP binding protein 2 | 54676 | 2.32365 [symbol] |
| 205014_at | HBP17 | Fibroblast growth factor binding protein 1 | 9982 | 3.21256 [symbol] |
| 208553_at | HIST1H1E | Histone 1, H1e | 3008 | 0.05285 [symbol] |
| 202934_at | HK2 | Hexokinase 2 | 3099 | 3.04435 [symbol] |
| 209377_s_at | HMGN3 | high mobility group nucleosomal binding domain 3 | 9324 | 0.30045 [symbol] |
| 213472_at | HNRPH1 | Heterogeneous nuclear ribonucleoprotein H1 (H) | 3187 | 0.327861 |
| 206858_s_at | HOXC6 | Data not found | 3223 | 0.231191 |
| 222881_at | HPSE | Heparanase | 10855 | 10.4687 [symbol] |
| 219403_s_at | HPSE | Heparanase | 10855 | 7.67497 [symbol] |
| 212983_at | HRAS | V-Ha-ras Harvey rat sarcoma viral oncogene homolog | 3265 | 50.0671 [symbol] |
| 201631_s_at | IER3 | Immediate early response 3 | 8870 | 13.3973 [symbol] |
| 206924_at | IL11 | Interleukin 11 | 3589 | 6.16771 [symbol] |
| 206172_at | IL13RA2 | Interleukin 13 receptor, alpha 2 | 3598 | 26.0753 [symbol] |
| 210118_s_at | IL1A | Interleukin 1, alpha | 3552 | 4.04548 [symbol] |
| 39402_at | IL1B | Interleukin 1, beta | 3553 | 3.43088 [symbol] |
| 205067_at | IL1B | Interleukin 1, beta | 3553 | 4.33704 [symbol] |
| 202859_x_at | IL8 | Interleukin 8 | 3576 | 2.99753 [symbol] |
| 202794_at | INPP1 | Inositol polyphosphate-1-phosphatase | 3628 | 2.02263 [symbol] |
| 223309_x_at | IPLA2(GAMMA) | Intracellular membrane-associated calcium-independent phospholipase A2 gamma | 50640 | 1.997961 |
| 228462_at | IRX2 | Iroquois homeobox protein 2 | 153572 | 0.31832 [symbol] |
| 205032_at | ITGA2 | Integrin, alpha 2 (CD49B, alpha 2 subunit of VLA-2 receptor) | 3673 | 5.54354 [symbol] |
| 201188_s_at | ITPR3 | Inositol 1,4,5-triphosphate receptor, type 3 | 3710 | 2.182901 |
| 201189_s_at | ITPR3 | Inositol 1,4,5-triphosphate receptor, type 3 | 3710 | 2.44663 [symbol] |
| 201473_at | JUNB | Jun B proto-oncogene | 3726 | 4.83143 [symbol] |
| 204678_s_at | KCNK1 | Potassium channel, subfamily K, member 1 | 3775 | 7.02525 [symbol] |
| 204679_at | KCNK1 | Potassium channel, subfamily K, member 1 | 3775 | 4.88500 [symbol] |
| 204401_at | KCNN4 | Potassium intermediate/small conductance calcium-activated | 3783 | 2.81128 [symbol] |
| | | channel, subfamily N, member 4 | | |
| 204882_at | KIAA0053 | Rho GTPase activating protein 25 | 9938 | 6.72199 [symbol] |
| 38149_at | KIAA0053 | Rho GTPase activating protein 25 | 9938 | 3.27802 [symbol] |
| 225611_at | KIAA0303 | Microtubule associated serine/threonine kinase family member 4 | 23227 | 3.00211 [symbol] |
| 41386_i_at | KIAA0346 | Jumonji domain containing 3 | 23135 | 4.70761 [symbol] |
| 212943_at | KIAA0528 | KIAA0528 gene product | 9847 | 0.32531 [symbol] |
| 226808_at | KIAA0543 | KIAA0543 protein | 23145 | 0.380111 |
| 213358_at | KIAA0802 | Data not found | 23255 | 0.31806 [symbol] |
| 229817_at | KIAA1281 | Zinc finger protein 608 | 57507 | 0.37455 [symbol] |
| 221778_at | KIAA1718 | KIAA1718 protein | 80853 | 2.56619 [symbol] |
| 225582_at | KIAA1754 | KIAA1754 | 85450 | 3.34972 [symbol] |
| 209212_s_at | KLF5 | Kruppel-like factor 5 (intestinal) | 688 | 3.33129 [symbol] |
| 212408_at | LAP1B | Lamina-associated polypeptide 1B | 26092 | 4.49603 [symbol] |
| 202067_s_at | LDLR | Low density lipoprotein receptor (familial hypercholesterolemia) | 3949 | 7.68000 [symbol] |
| 217173_s_at | LDLR | Low density lipoprotein receptor (familial hypercholesterolemia) | 3949 | 7.71913 [symbol] |
| 202068_s_at | LDLR | Low density lipoprotein receptor (familial hypercholesterolemia) | 3949 | 5.69336 [symbol] |
| 210732_s_at | LGALS8 | Lectin, galactoside-binding, soluble, 8 (galectin 8) | 3964 | 0.48203 [symbol] |
| 212658_at | LHFPL2 | Lipoma HMGIC fusion partner-like 2 | 10184 | 1.68390 [symbol] |
| 205266_at | LIF | Data not found | 3976 | 5.17972 [symbol] |
| 1558846_at | LOC119548 | Pancreatic lipase-related protein 3 | 119548 | 2.87385 [symbol] |
| 230323_s_at | LOC120224 | Transmembrane protein 45B | 120224 | 4.64963 [symbol] |
| 226726_at | LOC129642 | O-acyltransferase (membrane bound) domain containing 2 | 129642 | 3.512111 |
| 238058_at | LOC150381 | Data not found | 150381 | 0.36682 [symbol] |
| 228046_at | LOC152485 | Hypothetical protein LOC152485 | 152485 | 0.33288 [symbol] |
| 232158_x_at | LOC152519 | Hypothetical protein LOC152519 | 152519 | 6.37514 [symbol] |
| 229125_at | LOC163782 | Hypothetical protein LOC163782 | 163782 | 0.27441 [symbol] |
| 220317_at | LRAT | Lecithin retinol acyltransferase (phosphatidylcholine--retinol O-acyltransferase) | 9227 | 3.97767 [symbol] |
| 208433_s_at | LRP8 | Low density lipoprotein receptor-related protein 8, apolipoprotein e receptor | 7804 | 1.79253 [symbol] |
| 202626_s_at | LYN | V-yes-1 Yamaguchi sarcoma viral related oncogene homolog | 4067 | 0.34550 [symbol] |
| 228846_at | MAD | MAX dimerization protein 1 | 4084 | 4.93234 [symbol] |
| 226275_at | MAD | MAX dimerization protein 1 | 4084 | 3.63304 [symbol] |
| 223217_s_at | MAIL | Nuclear factor of kappa light polypeptide gene enhancer in B-cells inhibitor, zeta | 64332 | 2.82099 [symbol] |
| 208786_s_at | MAP1LC3B | Microtubule-associated protein 1 light chain 3 beta | 81631 | 3.520961 |
| 232138_at | MBNL2 | Muscleblind-like 2 (Drosophila) | 10150 | 0.20508 [symbol] |
| 200797_s_at | MCL1 | Myeloid cell leukemia sequence 1 (BCL2-related) | 4170 | 3.25108 [symbol] |
| 235374_at | MDH1 | Malate dehydrogenase 1, NAD (soluble) | 4190 | 0.48324 [symbol] |
| 235077_at | MEG3 | maternally expressed 3 | 55384 | 10.5318 [symbol] |
| 203417_at | MFAP2 | Microfibrillar-associated protein 2 | 4237 | 3.96641 [symbol] |
| 224480_s_at | MGC11324 | Hypothetical protein MGC11324 | 84803 | 2.99321 [symbol] |
| 215239_x_at | MGC12518 | Data not found | 90816 | 0.56858 [symbol] |
| 238741_at | MGC14128 | Hypothetical protein MGC14128 | 84985 | 6.34769 [symbol] |
| 229518_at | MGC16491 | Family with sequence similarity 46, member B | 115572 | 0.19213 [symbol] |
| 220949_s_at | MGC5242 | Hypothetical protein MGC5242 | 78996 | 0.49284 [symbol] |
| 203636_at | MID1 | Midline 1 (Opitz/BBB syndrome) | 4281 | 0.44911 [symbol] |
| 1557158_s_at | MLL3 | Data not found | 58508 | 0.420551 |
| 217279_x_at | MMP14 | Matrix metalloproteinase 14 (membrane-inserted) | 4323 | 6.49188 [symbol] |
| 202828_s_at | MMP14 | Matrix metalloproteinase 14 (membrane-inserted) | 4323 | 8.973361 |
| 160020_at | MMP14 | Matrix metalloproteinase 14 (membrane-inserted) | 4323 | 7.36443 [symbol] |
| 1553293_at | MRGX3 | G protein-coupled receptor MRGX3 | 117195 | 2.49595 [symbol] |
| 228527_s_at | MSCP | Mitochondrial solute carrier protein | 51312 | 10.1173 [symbol] |
| 212096_s_at | MTSG1 | Mitochondrial tumor suppressor 1 | 57509 | 0.331331 |
| 209124_at | MYD88 | Myeloid differentiation primary response gene (88) | 4615 | 2.639961 |
| 204823_at | NAV3 | Neuron navigator 3 | 89795 | 21.1442 [symbol] |
| 200632_s_at | NDRG1 | N-myc downstream regulated gene 1 | 10397 | 4.20954 [symbol] |
| 211467_s_at | NFIB | Nuclear factor I/B | 4781 | 0.33060 [symbol] |
| 205895_s_at | NOLC1 | Nucleolar and coiled-body phosphoprotein 1 | 9221 | 1.69418 [symbol] |
| 1553995_a_at | NT5E | 5′-nucleotidase, ecto (CD73) | 4907 | 4.85447 [symbol] |
| 203939_at | NT5E | 5′-nucleotidase, ecto (CD73) | 4907 | 5.39240 [symbol] |
| 206376_at | NTT73 | Solute carrier family 6, member 15 | 55117 | 2.76342 [symbol] |
| 200790_at | ODC1 | Ornithine decarboxylase 1 | 4953 | 12.5505 [symbol] |
| 202696_at | OSR1 | Oxidative-stress responsive 1 | 9943 | 3.633391 |
| 218736_s_at | PALMD | Palmdelphin | 54873 | 0.31391 [symbol] |
| 1555167_s_at | PBEF | Pre-B-cell colony enhancing factor 1 | 10135 | 2.98847 [symbol] |
| 227458_at | PDCD1LG1 | CD274 antigen | 29126 | 6.069811 |
| 223834_at | PDCD1LG1 | CD274 antigen | 29126 | 3.56404 [symbol] |
| 217997_at | PHLDA1 | Pleckstrin homology-like domain, family A, member 1 | 22822 | 3.37366 [symbol] |
| 218000_s_at | PHLDA1 | Pleckstrin homology-like domain, family A, member 1 | 22822 | 4.04616 [symbol] |
| 217996_at | PHLDA1 | Pleckstrin homology-like domain, family A, member 1 | 22822 | 3.05565 [symbol] |
| 209803_s_at | PHLDA2 | Pleckstrin homology-like domain, family A, member 2 | 7262 | 3.06347 [symbol] |
| 203691_at | PI3 | Protease inhibitor 3; skin-derived (SKALP) | 5266 | 9.705381 |
| 217864_s_at | PIAS1 | Protein inhibitor of activated STAT, 1 | 8554 | 0.41226 [symbol] |
| 203879_at | PIK3CD | Data not found | 5293 | 2.51997 [symbol] |
| 209193_at | PIM1 | Pim-1 oncogene | 5292 | 4.13447 [symbol] |
| 221577_x_at | PLAB | Growth differentiation factor 15 | 9518 | 3.79213 [symbol] |
| 210845_s_at | PLAUR | Plasminogen activator, urokinase receptor | 5329 | 9.36404 [symbol] |
| 211924_s_at | PLAUR | Plasminogen activator, urokinase receptor | 5329 | 11.9373 [symbol] |
| 214866_at | PLAUR | Plasminogen activator, urokinase receptor | 5329 | 2.79804 [symbol] |
| 213030_s_at | PLXNA2 | plexin A2 | 5362 | 2.86793 [symbol] |
| 215667_x_at | PMS2L6 | Data not found | 5384 | 0.49893 [symbol] |
| 209598_at | PNMA2 | Paraneoplastic antigen MA2 | 10687 | 2.78140 [symbol] |
| 214146_s_at | PPBP | Pro-platelet basic protein (chemokine (C—X—C motif) ligand 7) | 5473 | 57.8671 [symbol] |
| 201490_s_at | PPIF | Peptidylprolyl isomerase F (cyclophilin F) | 10105 | 2.59297 [symbol] |
| 201489_at | PPIF | Peptidylprolyl isomerase F (cyclophilin F) | 10105 | 3.45617 [symbol] |
| 202014_at | PPP1R15A | Protein phosphatase 1, regulatory (inhibitor) subunit 15A | 23645 | 8.48922 [symbol] |
| 37028_at | PPP1R15A | Protein phosphatase 1, regulatory (inhibitor) subunit 15A | 23645 | 5.72238 [symbol] |
| 215707_s_at | PRNP | Prion protein (p27-30) (Creutzfeld-Jakob disease, | 5621 | 3.00777 [symbol] |
| | | Gerstmann-Strausler-Scheinker syndrome, fatal far [symbol] | | |
| 227510_x_at | PRO1073 | Data not found | 29005 | 7.31426 [symbol] |
| 231735_s_at | PRO1073 | Data not found | 29005 | 0.296591 |
| 1554997_a_at | PTGS2 | Prostaglandin-endoperoxide synthase 2 (prostaglandin G/H synthase and cyclooxygenase) | 5743 | 25.9443 [symbol] |
| 204748_at | PTGS2 | Prostaglandin-endoperoxide synthase 2 (prostaglandin G/H synthase and cyclooxygenase) | 5743 | 20.7047 [symbol] |
| 211756_at | PTHLH | Parathyroid hormone-like hormone | 5744 | 4.67036 [symbol] |
| 210355_at | PTHLH | Parathyroid hormone-like hormone | 5744 | 4.41736 [symbol] |
| 1556773_at | PTHLH | Parathyroid hormone-like hormone | 5744 | 3.30276 [symbol] |
| 221840_at | PTPRE | Protein tyrosine phosphatase, receptor type, E | 5791 | 3.76078 [symbol] |
| 206157_at | PTX3 | Pentraxin-related gene, rapidly induced by IL-1 beta | 5806 | 8.98746 [symbol] |
| 214443_at | PVR | Poliovirus receptor | 5817 | 3.29373 [symbol] |
| 225189_s_at | RAPH1 | Ras association (RaIGDS/AF-6) and pleckstrin homology domains 1 | 65059 | 3.98712 [symbol] |
| 225188_at | RAPH1 | Ras association (RaIGDS/AF-6) and pleckstrin homology domains 1 | 65059 | 3.85497 [symbol] |
| 1553722_s_at | RNF152 | Ring finger protein 152 | 220441 | 0.146351 |
| 204133_at | RNU3IP2 | RNA, U3 small nucleolar interacting protein 2 | 9136 | 2.67640 [symbol] |
| 211181_x_at | RUNX1 | Runt-related transcription factor 1 (acute myeloid leukemia 1; aml1 oncogene) | 861 | 0.14529 [symbol] |
| 211182_x_at | RUNX1 | Runt-related transcription factor 1 (acute myeloid leukemia 1; aml1 oncogene) | 861 | 0.11277 [symbol] |
| 228923_at | S100A6 | S100 calcium binding protein A6 (calcyclin) | 6277 | 4.38041 [symbol] |
| 230333_at | SAT | Spermidine/spermine N1-acetyltransferase | 6303 | 4.64868 [symbol] |
| 201286_at | SDC1 | Syndecan 1 | 6382 | 8.69198 [symbol] |
| 201287_s_at | SDC1 | Syndecan 1 | 6382 | 5.06536 [symbol] |
| 202071_at | SDC4 | Syndecan 4 (amphiglycan, ryudocan) | 6385 | 3.41605 [symbol] |
| 234725_s_at | SEMA4B | Sema domain, immunoglobulin domain (Ig), transmembrane domain (TM) and short | 10509 | 2.54755 [symbol] |
| | | cytoplasmic domain, (semaph [symbol] | | |
| 46665_at | SEMA4C | Sema domain, immunoglobulin domain (Ig), transmembrane domain (TM) | 54910 | 3.52042 [symbol] |
| | | and short cytoplasmic domain, (semaph [symbol] | | |
| 219039_at | SEMA4C | Sema domain, immunoglobulin domain (Ig), transmembrane domain (TM) | 54910 | 4.31566 [symbol] |
| | | and short cytoplasmic domain, (semaph [symbol] | | |
| 212268_at | SERPINB1 | Serine (or cysteine) proteinase inhibitor, clade B (ovalbumin), member 1 | 1992 | 6.14074 [symbol] |
| 213572_s_at | SERPINB1 | Serine (or cysteine) proteinase inhibitor, clade B (ovalbumin), member 1 | 1992 | 3.78774 [symbol] |
| 228726_at | SERPINB1 | Serine (or cysteine) proteinase inhibitor, clade B (ovalbumin), member 1 | 1992 | 5.06481 [symbol] |
| 204614_at | SERPINB2 | Serine (or cysteine) proteinase inhibitor, clade B (ovalbumin), member 2 | 5055 | 11.5417 [symbol] |
| 209720_s_at | SERPINB3 | Serine (or cysteine) proteinase inhibitor, clade B (ovalbumin), member 3 | 6317 | 0.23453 [symbol] |
| 204855_at | SERPINB5 | Serine (or cysteine) proteinase inhibitor, clade B (ovalbumin), member 5 | 5268 | 2.86399 [symbol] |
| 223196_s_at | SESN2 | Sestrin 2 | 83667 | 1.79651 [symbol] |
| 223195_s_at | SESN2 | Sestrin 2 | 83667 | 3.04679 [symbol] |
| 242899_at | SESN3 | Sestrin 3 | 143686 | 0.16238 [symbol] |
| 209260_at | SFN | Stratifin | 2810 | 2.21416 [symbol] |
| 203625_x_at | SKP2 | S-phase kinase-associated protein 2 (p45) | 6502 | 0.13379 [symbol] |
| 202856_s_at | SLC16A3 | Solute carrier family 16 (monocarboxylic acid transporters), member 3 | 9123 | 6.62149 [symbol] |
| 201920_at | SLC20A1 | Solute carrier family 20 (phosphate transporter), member 1 | 6574 | 6.17375 [symbol] |
| 216236_s_at | SLC2A14 | Data not found | 144195 | 6.98069 [symbol] |
| 202499_s_at | SLC2A3 | Solute carrier family 2 (facilitated glucose transporter), member 3 | 6515 | 8.70822 [symbol] |
| 209453_at | SLC9A1 | Solute carrier family 9 (sodium/hydrogen exchanger), isoform 1 (antiporter, | 6548 | 3.09439 [symbol] |
| | | Na+/H+, amiloride sensitiv [symbol] | | |
| 209427_at | SMTN | Smoothelin | 6525 | 3.66808 [symbol] |
| 207390_s_at | SMTN | Smoothelin | 6525 | 3.40040 [symbol] |
| 230820_at | SMURF2 | SMAD specific E3 ubiquitin protein ligase 2 | 64750 | 3.04445 [symbol] |
| 210001_s_at | SOCS1 | Suppressor of cytokine signaling 1 | 8651 | 4.71057 [symbol] |
| 221489_s_at | SPRY4 | Sprouty homolog 4 (Drosophila) | 81848 | 4.45409 [symbol] |
| 1554671_a_at | SRRM2 | Serine/arginine repetitive matrix 2 | 23524 | 0.18824 [symbol] |
| 202440_s_at | ST5 | Suppression of tumorigenicity 5 | 6764 | 0.54559 [symbol] |
| 204729_s_at | STX1A | Syntaxin 1A (brain) | 6804 | 3.66517 [symbol] |
| 225544_at | TBX3 | T-box 3 (ulnar mammary syndrome) | 6926 | 4.32520 [symbol] |
| 216035_x_at | TCF7L2 | Data not found | 6934 | 0.37479 [symbol] |
| 209278_s_at | TFPI2 | Tissue factor pathway inhibitor 2 | 7980 | 25.5470 [symbol] |
| 205016_at | TGFA | Transforming growth factor, alpha | 7039 | 5.68073 [symbol] |
| 205015_s_at | TGFA | Transforming growth factor, alpha | 7039 | 13.8538 [symbol] |
| 220407_s_at | TGFB2 | Transforming growth factor, beta 2 | 7042 | 0.19218 [symbol] |
| 201447_at | TIA1 | TIA1 cytotoxic granule-associated RNA binding protein | 7072 | 0.52088 [symbol] |
| 201666_at | TIMP1 | Tissue inhibitor of metalloproteinase 1 (erythroid potentiating | 7076 | 5.20124 [symbol] |
| | | activity, collagenase inhibitor) | | |
| 1552648_a_at | TNFRSF10A | Tumor necrosis factor receptor superfamily, member 10a | 8797 | 5.04078 [symbol] |
| 231775_at | TNFRSF10A | Tumor necrosis factor receptor superfamily, member 10a | 8797 | 4.51113 [symbol] |
| 210405_x_at | TNFRSF10B | Tumor necrosis factor receptor superfamily, member 10b | 8795 | 3.57940 [symbol] |
| 218368_s_at | TNFRSF12A | Tumor necrosis factor receptor superfamily, member 12A | 51330 | 2.94312 [symbol] |
| 234734_s_at | TNRC6 | Trinucleotide repeat containing 6A | 27327 | 0.69259 [symbol] |
| 228834_at | TOB1 | Transducer of ERBB2, 1 | 10140 | 2.35168 [symbol] |
| 208901_s_at | TOP1 | Data not found | 7150 | 2.61498 [symbol] |
| 238688_at | TPM1 | Tropomyosin 1 (alpha) | 7168 | 0.17662 [symbol] |
| 213293_s_at | TRIM22 | Tripartite motif-containing 22 | 10346 | 0.41757 [symbol] |
| 215111_s_at | TSC22 | TSC22 domain family, member 1 | 8848 | 2.441881 |
| 226120_at | TTC8 | Tetratricopeptide repeat domain 8 | 123016 | 0.27249 [symbol] |
| 212242_at | TUBA1 | Data not found | 7277 | 2.95915 [symbol] |
| 209340_at | UAP1 | UDP-N-acteylglucosamine pyrophosphorylase 1 | 6675 | 3.48694 [symbol] |
| 221291_at | ULBP2 | UL16 binding protein 2 | 80328 | 2.07973 [symbol] |
| 203234_at | UPP1 | Uridine phosphorylase 1 | 7378 | 8.27180 [symbol] |
| 226029_at | VANGL2 | Vang-like 2 (van gogh, Drosophila) | 57216 | 0.29000 [symbol] |
| 212171_x_at | VEGF | Vascular endothelial growth factor | 7422 | 5.26283 [symbol] |
| 210513_s_at | VEGF | Vascular endothelial growth factor | 7422 | 4.34198 [symbol] |
| 211527_x_at | VEGF | Vascular endothelial growth factor | 7422 | 4.72168 [symbol] |
| 210512_s_at | VEGF | Vascular endothelial growth factor | 7422 | 3.47878 [symbol] |
| 1553993_s_at | WDR5 | WD repeat domain 5 | 11091 | 0.46692 [symbol] |
| 219836_at | ZBED2 | Zinc finger, BED domain containing 2 | 79413 | 4.25354 [symbol] |
| 201531_at | ZFP36 | Zinc finger protein 36, C3H type, homolog (mouse) | 7538 | 4.23412 [symbol] |
| 206579_at | ZNF192 | Zinc finger protein 192 | 7745 | 0.45102 [symbol] |
| 234608_at | — | Data not found | — | 11.6827 [symbol] |
| 226863_at | — | Data not found | — | 5.35537 [symbol] |
| 228314_at | — | Data not found | — | 3.88616 [symbol] |
| 239331_at | — | Data not found | — | 9.40224 [symbol] |
| 242509_at | — | Data not found | — | 3.707181 |
| 217608_at | — | Hypothetical LOC133993 | 133993 | 3.86433 [symbol] |
| 244025_at | — | Data not found | — | 5.71931 [symbol] |
| 240991_at | — | Data not found | — | 4.82194 [symbol] |
| 226034_at | — | Data not found | — | 4.57857 [symbol] |
| 230711_at | — | Data not found | — | 4.22249 [symbol] |
| 227755_at | — | Data not found | — | 3.66410 [symbol] |
| 1566968_at | — | Data not found | — | 19.5709 [symbol] |
| 227288_at | — | Hypothetical LOC133993 | 133993 | 2.58290 [symbol] |
| 208785_s_at | — | Data not found | — | 3.29382 [symbol] |
| 230973_at | — | Data not found | 374961 | 3.413311 |
| 225950_at | — | Data not found | — | 2.706131 |
| 225316_at | — | Data not found | — | 4.16493 [symbol] |
| 230778_at | — | Data not found | — | 2.32502 [symbol] |
| 211506_s_at | — | Data not found | — | 2.56361 [symbol] |
| 227057_at | — | Data not found | 374805 | 18.1159 [symbol] |
| 1558517_s_at | — | Data not found | — | 3.80787 [symbol] |
| 224606_at | — | Data not found | — | 2.686731 |
| 201861_s_at | — | Data not found | — | 2.58477 [symbol] |
| 216483_s_at | — | Data not found | — | 2.42522 [symbol] |
| 211620_x_at | — | Data not found | — | 0.22481 [symbol] |
| 229949_at | — | Data not found | — | 0.46297 [symbol] |
| 1568513_x_at | — | Data not found | — | 0.08123 [symbol] |
| 215071_s_at | — | Data not found | — | 0.28044 [symbol] |
| 232947_at | — | Data not found | — | 0.08281 [symbol] |
| 230779_at | — | Data not found | — | 0.19369 [symbol] |
| 232478_at | — | Data not found | — | 0.11705 [symbol] |
| 241464_s_at | — | Data not found | — | 0.30044 [symbol] |
| 229872_s_at | — | Data not found | — | 0.43056 [symbol] |
| 243712_at | — | Data not found | — | 0.27858 [symbol] |
| 1570425_s_at | — | Data not found | — | 0.22868 [symbol] |
| 236656_s_at | — | Data not found | — | 0.32802 [symbol] |
| 240245_at | — | Data not found | — | 0.18967 [symbol] |
| 216867_s_at | — | Data not found | 377602 | 0.11766 [symbol] |
| 232034_at | — | Data not found | — | 0.22081 [symbol] |
| 229004_at | — | Data not found | — | 0.188701 |
| 1559360_at | — | Data not found | — | 0.20979 [symbol] |
| 234951_s_at | — | Data not found | — | 0.20419 [symbol] |
| 227449_at | — | Data not found | — | 0.14967 [symbol] |
| 209908_s_at | — | Data not found | 376709 | 0.11659 [symbol] |
| Src | | | | |
| 213485_s_at | ABCC10 | ATP-binding cassette, sub-family C (CFTR/MRP), member 10 | 89845 | 0.68917 [symbol] |
| 201128_s_at | ACLY | ATP citrate lyase | 47 | 0.58744 [symbol] |
| 215867_x_at | AP1G1 | Adaptor-related protein complex 1, gamma 1 subunit | 164 | 0.64321 [symbol] |
| 201879_at | ARIH1 | Ariadne homolog, ubiquitin-conjugating enzyme E2 binding protein, 1 (Drosophila) | 25820 | 0.90244 [symbol] |
| 222667_s_at | ASH1L | Data not found | 55870 | 0.65957 [symbol] |
| 218796_at | C20orf42 | Chromosome 20 open reading frame 42 | 55612 | 0.72511 [symbol] |
| 206011_at | CASP1 | Caspase 1, apoptosis-related cysteine protease (interleukin 1, beta, convertase) | 834 | 0.81731 [symbol] |
| 213243_at | COH1 | Vacuolar protein sorting 13B (yeast) | 157680 | 0.65473 [symbol] |
| 221900_at | COL8A2 | Collagen, type VIII, alpha 2 | 1296 | 0.91510 [symbol] |
| 229666_s_at | CSTF3 | Data not found | 1479 | 0.591071 |
| 206414_s_at | DDEF2 | Development and differentiation enhancing factor 2 | 8853 | 0.76294 [symbol] |
| 213279_at | DHRS1 | Dehydrogenase/reductase (SDR family) member 1 | 115817 | 0.90491 [symbol] |
| 203301_s_at | DMTF1 | Cyclin D binding myb-like transcription factor 1 | 9988 | 0.83647 [symbol] |
| 213865_at | ESDN | Discoidin, CUB and LCCL domain containing 2 | 131566 | 0.65774 [symbol] |
| 225461_at | Eu-HMTase1 | Euchromatic histone methyltransferase 1 | 79813 | 0.66683 [symbol] |
| 209537_at | EXTL2 | Exostoses (multiple)-like 2 | 2135 | 0.77786 [symbol] |
| 218397_at | FANCL | Fanconi anemia, complementation group L | 55120 | 0.608521 |
| 1568680_s_at | FLJ21940 | YTH domain containing 2 | 64848 | 0.68372 [symbol] |
| 31874_at | GAS2L1 | Growth arrest-specific 2 like 1 | 10634 | 0.69758 [symbol] |
| 213056_at | GRSP1 | FERM domain containing 4B | 23150 | 0.56643 [symbol] |
| 206976_s_at | HSPH1 | Heat shock 105 kDa/110 kDa protein 1 | 10808 | 0.56081 [symbol] |
| 238933_at | IRS1 | Insulin receptor substrate 1 | 3667 | 0.54307 [symbol] |
| 235392_at | IRS1 | Insulin receptor substrate 1 | 3667 | 0.44403 [symbol] |
| 213352_at | KIAA0779 | Transmembrane and coiled-coil domains 1 | 23023 | 0.73246 [symbol] |
| 212492_s_at | KIAA0876 | Jumonji domain containing 2B | 23030 | 0.952351 |
| 213069_at | KIAA1237 | HEG homolog 1 (zebrafish) | 57493 | 0.50046 [symbol] |
| 219181_at | LIPG | Lipase, endothelial | 9388 | 0.54825 [symbol] |
| 231866_at | LNPEP | leucyl/cystinyl aminopeptidase | 4012 | 0.60419 [symbol] |
| 229582_at | LOC125476 | Chromosome 18 open reading frame 37 | 125476 | 0.60270 [symbol] |
| 202245_at | LSS | Lanosterol synthase (2,3-oxidosqualene-lanosterol cyclase) | 4047 | 0.64921 [symbol] |
| 202569_s_at | MARK3 | MAP/microtubule affinity-regulating kinase 3 | 4140 | 0.81434 [symbol] |
| 242082_at | MMAB | Methylmalonic aciduria (cobalamin deficiency) type B | 326625 | 1.25774 [symbol] |
| 213164_at | MRPS6 | Mitochondrial ribosomal protein S6 | 64968 | 0.72744 [symbol] |
| 37028_at | PPP1R15A | Protein phosphatase 1, regulatory (inhibitor) subunit 15A | 23645 | 2.24867 [symbol] |
| 226065_at | PRICKLE1 | Prickle-like 1 (Drosophila) | 144165 | 0.74535 [symbol] |
| 1552797_s_at | PROM2 | Prominin 2 | 150696 | 0.57989 [symbol] |
| 1556773_at | PTHLH | Parathyroid hormone-like hormone | 5744 | 0.57204 [symbol] |
| 211756_at | PTHLH | Parathyroid hormone-like hormone | 5744 | 0.65821 [symbol] |
| 206591_at | RAG1 | Recombination activating gene 1 | 5896 | 2.54153 [symbol] |
| 212044_s_at | RPL27A | Data not found | 6157 | 2.13058 [symbol] |
| V200908_s_at | RPLP2 | Ribosomal protein, large P2 | 6181 | 3.07911 [symbol] |
| 213350_at | RPS11 | Ribosomal protein S11 | 6205 | 4.38741 [symbol] |
| 202648_at | RPS19 | Ribosomal protein S19 | 6223 | 3.21199 [symbol] |
| 209773_s_at | RRM2 | Ribonucleotide reductase M2 polypeptide | 6241 | 0.72509 [symbol] |
| 213262_at | SACS | Spastic ataxia of Charlevoix-Saguenay (sacsin) | 26278 | 0.72051 [symbol] |
| 224250_s_at | SBP2 | SECIS binding protein 2 | 79048 | 0.80073 [symbol] |
| 204614_at | SERPINB2 | Serine (or cysteine) proteinase inhibitor, clade B (ovalbumin), member 2 | 5055 | 0.56926 [symbol] |
| 204404_at | SLC12A2 | Solute carrier family 12 (sodium/potassium/chloride transporters), member 2 | 6558 | 0.82319 [symbol] |
| 212560_at | SORL1 | Data not found | 6653 | 0.60806 [symbol] |
| 1558211_s_at | SRC | V-src sarcoma (Schmidt-Ruppin A-2) viral oncogene homolog (avian) | 6714 | 26.3231 [symbol] |
| 221284_s_at | SRC | V-src sarcoma (Schmidt-Ruppin A-2) viral oncogene homolog (avian) | 6714 | 5.32298 [symbol] |
| 202506_at | SSFA2 | Sperm specific antigen 2 | 6744 | 0.68778 [symbol] |
| 201737_s_at | TEB4 | Membrane-associated ring finger (C3HC4) 6 | 10299 | 0.64972 [symbol] |
| 201447_at | TIA1 | TIA1 cytotoxic granule-associated RNA binding protein | 7072 | 0.67273 [symbol] |
| 224321_at | TMEFF2 | Transmembrane protein with EGF-like and two follistatin-like domains 2 | 23671 | 4.171491 |
| 202643_s_at | TNFAIP3 | Tumor necrosis factor, alpha-induced protein 3 | 7128 | 0.55537 [symbol] |
| 220687_at | TRRAP | Transformation/transcription domain-associated protein | 8295 | 1.24000 [symbol] |
| 212928_at | TSPYL4 | TSPY-like 4 | 23270 | 0.63264 [symbol] |
| 1554021_a_at | ZNF325 | Data not found | 51711 | 0.621751 |
| 219571_s_at | ZNF325 | Data not found | 51711 | 0.78162 [symbol] |
| 204847_at | ZNF-U69274 | Zinc finger and BTB domain containing 11 | 27107 | 0.72777 [symbol] |
| 241617_x_at | — | Data not found | — | 2.12972 [symbol] |
| 229101_at | — | Data not found | — | 0.94339 [symbol] |
| 225640_at | — | Data not found | — | 0.846531 |
| 212435_at | — | Data not found | — | 0.71735 [symbol] |
| 235423_at | — | Data not found | — | 0.64546 [symbol] |
| 230304_at | — | Data not found | — | 0.39179 [symbol] |
| 228955_at | — | Data not found | — | 0.58012 [symbol] |
| 1556006_s_at | — | Data not found | — | 0.65433 [symbol] |
| 227921_at | — | Data not found | — | 0.53322 [symbol] |
| 1556499_s_at | — | Data not found | — | 0.59122 [symbol] |
| 236251_at | — | Data not found | — | 0.59152 [symbol] |
| 1568408_x_at | — | Data not found | — | 0.70623 [symbol] |
| β-catenin | | | | |
| 225098_at | ABI-2 | Abl interactor 2 | 10152 | 0.853191 |
| 218150_at | ARL5 | ADP-ribosylation factor-like 5 | 26225 | 0.86884 [symbol] |
| 222667_s_at | ASH1L | Data not found | 55870 | 0.72480 [symbol] |
| 208859_s_at | ATRX | Alpha thalassemia/mental retardation syndrome X-linked | 546 | 0.78315 [symbol] |
| | | (RAD54 homolog, S. cerevisiae) | | |
| 222696_at | AXIN2 | Axin 2 (conductin, axil) | 8313 | 6.45354 [symbol] |
| 60474_at | C20orf42 | Chromosome 20 open reading frame 42 | 55612 | 0.74119 [symbol] |
| 218796_at | C20orf42 | Chromosome 20 open reading frame 42 | 55612 | 0.81536 [symbol] |
| 212996_s_at | C21orf108 | Chromosome 21 open reading frame 108 | 9875 | 0.75222 [symbol] |
| 212177_at | C6orf111 | Chromosome 6 open reading frame 111 | 25957 | 0.71391 [symbol] |
| 204048_s_at | C6orf56 | Phosphatase and actin regulator 2 | 9749 | 0.80934 [symbol] |
| 1555945_s_at | C9orf10 | Chromosome 9 open reading frame 10 | 23196 | 0.79636 [symbol] |
| 1555920_at | CBX3 | Chromobox homolog 3 (HP1 gamma homolog, Drosophila) | 11335 | 0.75054 [symbol] |
| 236241_at | CGI-125 | Mediator of RNA polymerase II transcription, subunit 31 homolog (yeast) | 51003 | 0.71621 [symbol] |
| 211343_s_at | COL13A1 | Collagen, type XIII, alpha 1 | 1305 | 0.61354 [symbol] |
| 221900_at | COL8A2 | Collagen, type VIII, alpha 2 | 1296 | 0.89910 [symbol] |
| 215646_s_at | CSPG2 | Chondroitin sulfate proteoglycan 2 (versican) | 1462 | 0.63490 [symbol] |
| 209257_s_at | CSPG6 | Chondroitin sulfate proteoglycan 6 (bamacan) | 9126 | 0.73471 [symbol] |
| 206504_at | CYP24A1 | Cytochrome P450, family 24, subfamily A, polypeptide 1 | 1591 | 3.638601 |
| 223139_s_at | DHX36 | DEAH (Asp-Glu-Ala-His) box polypeptide 36 | 170506 | 0.84394 [symbol] |
| 229115_at | DNCH1 | Dynein, cytoplasmic, heavy polypeptide 1 | 1778 | 0.68153 [symbol] |
| 209457_at | DUSP5 | Dual specificity phosphatase 5 | 1847 | 0.70328 [symbol] |
| 212420_at | ELF1 | E74-like factor 1 (ets domain transcription factor) | 1997 | 0.70032 [symbol] |
| 200842_s_at | EPRS | Glutamyl-prolyl-tRNA synthetase | 2058 | 0.711191 |
| 203255_at | FBXO11 | F-box protein 11 | 80204 | 0.83511 [symbol] |
| 226799_at | FGD6 | FYVE, RhoGEF and PH domain containing 6 | 55785 | 0.70437 [symbol] |
| 225021_at | FLJ10697 | Zinc finger protein 532 | 55205 | 0.78984 [symbol] |
| 235388_at | FLJ12178 | Data not found | 80205 | 0.72934 [symbol] |
| 222760_at | FLJ14299 | Hypothetical protein FLJ14299 | 80139 | 2.79584 [symbol] |
| 232094_at | FLJ22557 | Chromosome 15 open reading frame 29 | 79768 | 0.71283 [symbol] |
| 227475_at | FOXQ1 | Forkhead box Q1 | 94234 | 1.51528 [symbol] |
| 210178_x_at | FUSIP1 | FUS interacting protein (serine/arginine-rich) 1 | 10772 | 0.80834 [symbol] |
| 222834_s_at | GNG12 | Guanine nucleotide binding protein (G protein), gamma 12 | 55970 | 0.59954 [symbol] |
| 225097_at | HIPK2 | Homeodomain interacting protein kinase 2 | 28996 | 0.78873 [symbol] |
| 225116_at | HIPK2 | Homeodomain interacting protein kinase 2 | 28996 | 0.80948 [symbol] |
| 210118_s_at | IL1A | Interleukin 1, alpha | 3552 | 0.62238 [symbol] |
| 208953_at | KIAA0217 | KIAA0217 | 23185 | 0.87479 [symbol] |
| 212355_at | KIAA0323 | KIAA0323 | 23351 | 0.846491 |
| 213352_at | KIAA0779 | Transmembrane and coiled-coil domains 1 | 23023 | 0.71413 [symbol] |
| 1554260_a_at | KIAA0826 | Data not found | 23045 | 0.65296 [symbol] |
| 216563_at | KIAA0874 | Ankyrin repeat domain 12 | 23253 | 0.71910 [symbol] |
| 212492_s_at | KIAA0876 | Jumonji domain containing 2B | 23030 | 0.80413 [symbol] |
| 213478_at | KIAA1026 | Kazrin | 23254 | 0.856901 |
| 212794_s_at | KIAA1033 | KIAA1033 | 23325 | 0.72300 [symbol] |
| 235009_at | KIAA1327 | KIAA1327 protein | 57219 | 0.89735 [symbol] |
| 223380_s_at | LATS2 | LATS, large tumor suppressor, homolog 2 (Drosophila) | 26524 | 0.81979 [symbol] |
| 212692_s_at | LRBA | LPS-responsive vesicle trafficking, beach and anchor containing | 987 | 0.81700 [symbol] |
| 1558173_a_at | LUZP1 | leucine zipper protein 1 | 7798 | 0.79562 [symbol] |
| 229846_s_at | MAPKAP1 | Mitogen-activated protein kinase associated protein 1 | 79109 | 0.908201 |
| 222728_s_at | MGC5306 | Hypothetical protein MGC5306 | 79101 | 0.647211 |
| 207700_s_at | NCOA3 | Nuclear receptor coactivator 3 | 8202 | 0.75129 [symbol] |
| 213328_at | NEK1 | NIMA (never in mitosis gene a)-related kinase 1 | 4750 | 0.82268 [symbol] |
| 203304_at | NMA | BMP and activin membrane-bound inhibitor homolog (Xenopus laevis) | 25805 | 1.52865 [symbol] |
| 211671_s_at | NR3C1 | Nuclear receptor subfamily 3, group C, member 1 (glucocorticoid receptor) | 2908 | 0.75247 [symbol] |
| 229422_at | NRD1 | Nardilysin (N-arginine dibasic convertase) | 4898 | 0.90202 [symbol] |
| 244677_at | PER1 | Period homolog 1 (Drosophila) | 5187 | 0.74427 [symbol] |
| 226094_at | PIK3C2A | Phosphoinositide-3-kinase, class 2, alpha polypeptide | 5286 | 0.69776 [symbol] |
| 207002_s_at | PLAGL1 | Data not found | 5325 | 0.74302 [symbol] |
| 209318_x_at | PLAGL1 | Data not found | 5325 | 0.66435 [symbol] |
| 219024_at | PLEKHA1 | Pleckstrin homology domain containing, family A | 59338 | 0.71952 [symbol] |
| | | (phosphoinositide binding specific) member 1 | | |
| 210355_at | PTHLH | Parathyroid hormone-like hormone | 5744 | 0.56397 [symbol] |
| 212263_at | QKI | Quaking homolog, KH domain RNA binding (mouse) | 9444 | 0.81747 [symbol] |
| 235209_at | RPESP | Data not found | 157869 | 1.59688 [symbol] |
| 212044_s_at | RPL27A | Data not found | 6157 | 1.71579 [symbol] |
| 213350_at | RPS11 | Ribosomal protein S11 | 6205 | 3.04174 [symbol] |
| 202648_at | RPS19 | Ribosomal protein S19 | 6223 | 2.39557 [symbol] |
| 224250_s_at | SBP2 | SECIS binding protein 2 | 79048 | 0.79137 [symbol] |
| 222747_s_at | SCML1 | Sex comb on midleg-like 1 (Drosophila) | 6322 | 0.77899 [symbol] |
| 1569594_a_at | SDCCAG1 | Serologically defined colon cancer antigen 1 | 9147 | 0.86647 [symbol] |
| 244287_at | SFRS12 | Splicing factor, arginine/serine-rich 12 | 140890 | 0.86284 [symbol] |
| 213850_s_at | SFRS2IP | Splicing factor, arginine/serine-rich 2, interacting protein | 9169 | 0.82759 [symbol] |
| 206108_s_at | SFRS6 | Splicing factor, arginine/serine-rich 6 | 6431 | 0.55726 [symbol] |
| 210057_at | SMG1 | PI-3-kinase-related kinase SMG-1 | 23049 | 0.69607 [symbol] |
| 203509_at | SORL1 | Data not found | 6653 | 0.82568 [symbol] |
| 212560_at | SORL1 | Data not found | 6653 | 0.63674 [symbol] |
| 222122_s_at | THOC2 | THO complex 2 | 57187 | 0.85999 [symbol] |
| 212994_at | THOC2 | THO complex 2 | 57187 | 0.75491 [symbol] |
| 202643_s_at | TNFAIP3 | Tumor necrosis factor, alpha-induced protein 3 | 7128 | 0.59005 [symbol] |
| 208901_s_at | TOP1 | Data not found | 7150 | 0.80643 [symbol] |
| 208900_s_at | TOP1 | Data not found | 7150 | 0.85890 [symbol] |
| 203147_s_at | TRIM14 | Tripartite motif-containing 14 | 9830 | 1.04452 [symbol] |
| 214814_at | YT521 | Splicing factor YT521-B | 91746 | 0.60367 [symbol] |
| 222227_at | ZNF236 | Zinc finger protein 236 | 7776 | 0.15922 [symbol] |
| 1555673_at | — | Data not found | — | 2.663031 |
| 241617_x_at | — | Data not found | — | 1.68804 [symbol] |
| 241464_s_at | — | Data not found | — | 0.76851 [symbol] |
| 217277_at | — | Data not found | — | 2.41938 [symbol] |
| 228315_at | — | Data not found | — | 0.79904 [symbol] |
| 233204_at | — | Data not found | — | 0.68806 [symbol] |
| 244075_at | — | Data not found | — | 0.70613 [symbol] |
| 201865_x_at | — | Data not found | — | 0.85930 [symbol] |
| 229958_at | — | Data not found | 286088 | 0.71001 [symbol] |
| 1557081_at | — | Data not found | — | 0.59551 [symbol] |
| 1560318_at | — | Data not found | — | 0.55048 [symbol] |
| 228180_at | — | Data not found | — | 0.76706 [symbol] |
| 1568408_x_at | — | Data not found | — | 0.62731 [symbol] |
| 1562416_at | — | Data not found | — | 0.72989 [symbol] |
| 232231_at | — | Data not found | — | 1.36253 [symbol] |
| 213637_at | — | Data not found | — | 0.78995 [symbol] |

[symbol]

indicates data missing or illegible when filed

TABLE 2
Ras mutation status in NSCLC samples.
PTID CellType Ras_prediction Ras
mutation

| | | | |
|---|---|---|---|
| 01-534--S | 0 | n | |
| 98-1277--S | 0 | n | |
| 99-77--S | 0 | n | |
| 99-728--S | 0 | n | |
| 99-830--S | 0 | n | |
| 98-320--S | 0.0000001 | n | |
| 98-506--S | 0.0000001 | n | |
| 98-1293--S | 0.0000001 | n | |
| 98-1296--A | 0.0000001 | n | |
| 99-692--S | 0.0000001 | n | |
| 98-853--S | 0.0000002 | n | |
| 99-706--S | 0.0000003 | n | |
| 99-927--S | 0.0000005 | n | |
| 99-301--S | 0.0000006 | n | |
| 98-292--S | 0.0000011 | n | |
| 97-829--S | 0.0000018 | n | |
| 00-151--S | 0.0000039 | n | |
| 00-550--S | 0.0000083 | n | |
| 01-284--S | 0.0000304 | n | |
| 97-1027--A | 0.0000484 | n | |
| 00-315--S | 0.0000556 | n | |
| 98-401--S | 0.000159 | n | |
| 00-452--S | 0.0001954 | n | |
| 98-933--S | 0.0008946 | n | |
| 97-666--S | 0.0011485 | n | |
| 00-253--A | 0.0032797 | n | |
| 00-1059--S | 0.0040104 | n | |
| 97-608--S | 0.0047135 | n | |
| 97-403--S | 0.0061926 | n | |
| 98-375--S | 0.0793839 | n | |
| 00-440--S | 0.0967915 | n | |
| 97-587--5 | 0.2257309 | n | |
| 98-152--A | 0.4123361 | n | |
| 97-949--S | 0.9681779 | n | |
| 10-00--S | 0.9775212 | n | |
| 98-417--A | 0.9777897 | n | |
| 00-827--S | 0.9899805 | n | |
| 96-3--A | 0.9938232 | n | |
| 99-1067--S | 0.9960476 | n | |
| 98-197--A | 0.9977215 | n | |
| 98-679--A | 0.9988883 | n | |
| 00-334--A | 0.9996112 | n | |
| 98-1146--A | 0.9997253 | n | |
| 00-479--A | 0.9997574 | n | |
| 97-1026--S | 0.9998406 | n | |
| 00-327--S | 0.9999319 | n | |
| 99-440--A | 0.9999847 | n | |
| 98-821--A | 0.9999914 | n | |
| 00-1072--A | 0.9999959 | n | |
| 98-1063--A | 0.9999979 | n | |
| 98-1216--A | 0.9999979 | n | |
| 98-543--A | 0.9999987 | n | |
| 99-137--A | 0.9999989 | n | |
| 99-1033--A | 0.999999 | n | |
| 00-909--A | 0.9999993 | n | |
| 01-646--A | 0.9999993 | n | |
| 98-683--A | 0.9999994 | n | |
| 01-369--S | 0.9999998 | n | |
| 98-438--A | 0.9999998 | n | |
| 99-671 --A | 0.9999999 | n | |
| 00-145--A | 1 | n | |
| 98-657--A | 1 | n | |
| 98-956--A | 1 | n | |
| 98-691--A | 0.9941423 | y | GGT > AGT |
| 98-723--A | 0.9991708 | y | GGT > TGT |
| 98-771--A | 0.9995594 | y | GGT > TGT |
| 96-353--A | 0.9996714 | y | GGT > TGT |
| 00-941--A | 0.9999252 | y | ND |
| 01-331--A | 0.9999722 | y | GGT > TGT |
| 99-1017--A | 0.9999896 | y | GGT > GCT |
| 98-711--A | 0.9999908 | y | GGT > GTT |
| 98-967--A | 0.9999985 | y | GGT > TGT |
| 00-703--A | 0.9999999 | y | GGT > TGT |
| 98-1014--A | 1 | y | GGT > TGT |
| | | % mut overall | 0.148648649 |
| | | % mut adeno | 0.289473684 |

| | Relative | Predicted | Relative | Predicted | Relative | Predicted | Relative β- | Predicted β- | Relative | Predicted |
|---|---|---|---|---|---|---|---|---|---|---|
| | E2F3 | E2F3 | Myc | Myc | phospho-Src | Src | catenin | catenin | Ras | Ras |
| | Expression | Activity | Expression | Activity | Expression | Activity | Expression | Activity | Activity | Activity |
| BT-483 | 1.1 | 11.3 | 22.2 | 12.7 | 49.9 | 57.5 | 42.8 | 36.4 | 10 | 50.8 |
| MCF7 | 3.7 | 5.7 | 27.2 | 11.9 | 32.7 | 43.8 | 12.8 | 24.2 | 52.4 | 56.3 |
| T47-D | 5.5 | 5.2 | 25.5 | 18.5 | 32.6 | 50.3 | 51 | 35.6 | 37.6 | 47.1 |
| BT-474 | 7.3 | 4.4 | 48.8 | 22.2 | 31.1 | 48.4 | 29.6 | 25.5 | 71.3 | 53.1 |
| SKBR3 | 8.9 | 8 | 40.1 | 34.4 | 37.4 | 44 | 0 | 29.3 | 84.2 | 58.1 |
| BT-20 | 12.4 | 25.3 | 41.1 | 21.6 | 38 | 51.7 | 60.7 | 29.9 | 63.6 | 58.4 |
| MDA-MB-435s | 100 | 87.4 | 95.1 | 60.6 | 100 | 69.1 | 25.6 | 43.5 | 25.3 | 54.6 |
| ZR-75 | 4.2 | 13.6 | 20.1 | 21.7 | 41.6 | 46.6 | 56.8 | 22.8 | 22 | 68.3 |
| MDA-MB-231 | 17.3 | 87.8 | 84.7 | 51.7 | 51.2 | 71 | 29.2 | 60 | 100 | 79.1 |
| BT-549 | 56 | 87.8 | 100 | 74.3 | 92.8 | 60.7 | 86 | 66.4 | 8.2 | 65.6 |
| MDA-MB-361 | 2.4 | 7.1 | 31 | 11.5 | 17 | 47.4 | 63.7 | 21 | 54.8 | 62.1 |
| HCC1143 | 9.2 | 34.2 | 81.6 | 71.9 | 3.7 | 36 | 100 | 57.2 | 20.2 | 58.2 |
| HS578t | 56.5 | 95.7 | 17.9 | 59.7 | 29.2 | 55.9 | 69.7 | 65 | 13 | 42.5 |
| HCC38 | 4.9 | 66.7 | 36.6 | 28.1 | 6.3 | 38.2 | 98.6 | 43.7 | 0 | 42 |
| CAMA1 | 4.3 | 4.9 | 15.1 | 16.8 | 0 | 42.7 | 26 | 25.4 | 85.7 | 59.8 |
| MDA-MB-157 | 95.8 | 94.9 | 46.7 | 32.7 | 60.9 | 64.6 | 42.1 | 59.2 | 66.6 | 48.3 |
| HCC1806 | 4.7 | 45.4 | 59.3 | 58.9 | 32.9 | 35.8 | 104.8 | 57.2 | 18.8 | 71 |
| MDA-MB-453 | 2.2 | 7.7 | 0 | 35.4 | 10.1 | 50.5 | 10.6 | 30 | 6.8 | 65.3 |
| HCC1428 | 0 | 74.5 | 40.9 | 90 | 2.8 | 36.9 | 49 | 84.5 | 10.8 | 63.7 |

| | | | | | |
|---|---|---|---|---|---|
| Pearson Correlation | 0.0006** | 0.0061** | <0.0001*** | 0.07 | 0.36 |
| (two-tailed p-value) | | | | | |

*to quantitate Western blot analyses, the average intensity value of each fixed area is measured. These values are presented as % relative to highest value.

The following attached documents, cited throughout the specification, are incorporated in their entirety by reference:

### REFERENCES

1. Fearon, E. R. & Vogelstein, B. A genetic model for colorectal tumorigenesis. Cell 17, 671-674 (1990).
2. Hanahan, D. & Weinberg, R. A. The Hallmarks of Cancer. Cell 100, 57-70 (2000).
3. Sherr, C. J. Cancer cell cycles. Science 274, 1672-1677 (1996).
4. Ramaswamy, S. & Golub, T. R. DNA microarrays in clinical oncology. J. Clin. Oncol. 20, 1932-1941 (2002).
5. Lamb, J. et al. A mechanism of cyclin D1 action encoded in the patterns of gene expression in human cancer. Cell 114, 323-334 (2003).
6. Huang, E. et al. Gene expression phenotypic models that predict the activity of oncogenic pathways. Nature Genet. 34, 226-230 (2003).
7. Black, E. P. et al. Distinct gene expression phenotypes of cells lacking Rb and Rb family members. Cancer Res. 63, 3716-3723 (2003).
8. Segal, E., Friedman, N., Koller, D. & Regev, A. A module map showing conditional activity of expression modules in cancer. Nature Genetics 36, 1090-1098 (2004).
9. Rhodes, D. R. et al. Large-scale meta-analysis of cancer microarray data identifies common transcriptional profiles of neoplastic transformation and progression. Proc Natl Acad Sci USA 101, 9309-9314 (2004).
10. Ramaswamy, S., Ross, K. N., Lander, E. S. & Golub, T. R. A molecular signature of metastasis in primary solid tumors. Nature Genetics 33, 59-54 (2003).
11. Mootha, V. K. et al. PGC-1alpha-responsive genes involved in oxidative phosphorylation are coordinately downregulated in human diabetes. Nat. Genet. 34, 267-273 (2003).
12. West, M. et al. Predicting the clinical status of human breast cancer by using gene expression profiles. Proc Natl Acad Sci USA 98, 11462-11467 (2001).
13. D'Crus, C. M. et al. c-MYC induces mammary tumorigenesis by means of a preferred pathway involving spontaneous Kras2 mutations. Nat. Med. 7, 235-239 (2001).
14. Sweet-Cordero, A. et al. An oncogenic KRAS2 expression signature identified by cross-species gene expression analysis. Nat. Genet. 37, 48-54 (2005).
15. Rodenhuis, S. et al. Mutational activation of the K-ras oncogene and the effect of chemotherapy in advanced adenocarcinoma of the lung: a prospective study. J. Clin. Oncol. 15, 285-291 (1997).
16. Salgia, R. & Skarin, A. T. Molecular abnormalities in lung cancer. J. Clin. Oncol. 16, 1207-1217 (1998).
17. Cory, A. H. Use of an aqueous soluble tetrazolium/formazan assay for cell growth assays in culture. Cancer Commun. 3, 207-212 (1991).
18. Riss, T. L. & A., M. R. Comparison of MTT, Xtt, and a novel tetrazolium compound for MTS for in vitro proliferation and chemosensitivity assays. Mol. Biol. Cell 3, 184a (1993).
19. Stampfer, M. R. & Yaswen, P. Culture systems for study of human mammary epithelial cell proliferation, differentiation, and transformation. Cancer Surv. 18, 7-34 (1993).
20. Huang, E. et al. Gene expression predictors of breast cancer outcomes. Lancet 361, 1590-1596 (2003).
21. Irizarry, R. A. et al. Exploration, normalization, and summaries of high density oligonucleotide array probe level data. Biostatistics in press (2004).
22. Bolstad, B. M., Irizarry, R. A., Astrand, M. & Speed, T. P. A comparison of normalization methods for high density oligonucleotide array data based on variance and bias. Bioinformatics 19, 185-193 (2003).
23. Eisen, M. B., Spellman, P. T., Brown, P. O. & Botstein, D. Cluster analysis and display of genome-wide expression patterns. 95, 14863-14868 (1998).
24. Mitsudomi, T. et al. Mutations of ras genes distinguish a subset of non-small-cell lung cancer cell lines from small-cell lung cancer cell lines. Oncogene 6, 1353-1362 (1991).

1. A method of estimating the efficacy of a therapeutic agent in treating a disorder in a subject, wherein the therapeutic agent regulates a pathway, said method comprising:
(a) determining the expression levels of multiple genes in a sample from a subject; and
(b) detecting the presence of pathway deregulation by comparing the expression levels of the genes to a reference profile indicative of pathway deregulation,
wherein the presence of pathway deregulation in step (b) indicates that the therapeutic agent is estimated to be effective in treating the disorder in the subject.

2. A method of estimating the efficacy of two or more therapeutic agents in treating a disorder in a subject, wherein the therapeutic agents each regulate a different pathway, said method comprising:
(a) determining the expression levels of multiple genes in a sample from a subject; and
(b) detecting the presence of pathway deregulation in each different pathway by comparing the expression levels of the genes to one or more reference profiles indicative of pathway deregulation,
wherein the presence of pathway deregulation in step (b) in the different pathways indicates that the therapeutic agent is estimated to be effective in treating the disorder in the subject.

3. The method of claim 1, wherein said sample is diseased tissue.

4. The method of claim 1, wherein said sample is a tumor sample.

5. The method of claim 4, wherein said tumor is selected from a breast tumor, an ovarian tumor, and a lung tumor.

6. The method of claim 1, wherein said therapeutic agents are selected from a farnesyl transferase inhibitor, a farnesylthiosalicylic acid, and a Src inhibitor.

7. The method of claim 1, wherein said pathways are selected from RAS, SRC, MYC, E2F, and β-catenin pathways.

8. The method of claim 1, wherein the measure of efficacy of a therapeutic agent is selected from the group consisting of disease-specific survival, disease-free survival, tumor recurrence, therapeutic response, tumor remission, and metastasis inhibition.

9. The method of claim 1, wherein step (b) comprises detecting the presence of pathway deregulation in the different pathways by using supervised classification methods of analysis.

10. The method of claim 1, wherein step (b) comprises:
(i) comparing samples with known deregulated pathways to controls to generate signatures; and
(ii) comparing the expression profile from the subject sample to the said signatures to indicate pathway deregulation.

11. A method of determining the deregulation status of multiple pathways in a tumor sample, said method comprising:
(a) obtaining an expression profile for said sample; and
(b) comparing said obtained expression profile to a reference profile to determine deregulation status of said pathways.

12. The method of claim 11, wherein the deregulation status of the pathways is hyperactivation.

13. The method of claim 11, wherein the deregulation status of the pathways is hypoactivation.

14. A method of estimating the efficacy of a therapeutic agent in treating cancer cells, wherein the therapeutic agent regulates a pathway, said method comprising:
(a) determining the expression levels of multiple genes in samples from a subject; and
(b) detecting the presence of pathway deregulation by comparing the expression levels of the genes to a reference profile indicative of pathway deregulation,
wherein the presence of pathway deregulation in step (b) indicates that the therapeutic agent is estimated to be effective in treating the cancer cells.

15. A method of using pathway signatures to analyze a large collection of human tumor samples to obtain profiles of the status of multiple pathways in said tumors, said method comprising:
(a) determining gene expression profiles from tumor samples; and
(b) identifying patterns of pathway deregulation by comparison of expression profiles with reference profiles.

16. A method of treating a subject afflicted with cancer, said method comprising:
(a) identifying a pathway that is deregulated in a tumor sample;
(b) selecting a therapeutic agent known to modulate the activity level of the pathway; and
(c) administering to the subject an effective amount of the therapeutic agent,
thereby treating the subject afflicted with cancer.

17. A method of treating a subject afflicted with cancer, said method comprising:
(a) identifying two or more pathways that are deregulated in a tumor sample;
(b) selecting a therapeutic agent known to modulate the activity level of each pathway; and
(c) administering to the subject an effective amount of the therapeutic agents,
thereby treating the subject afflicted with cancer.

18. The method of claim 16, wherein a therapeutic agent is a combination of two or more therapeutic agents.

19. The method of claim 16, wherein step (a) comprises:
(i) obtaining an expression profile from said sample; and
(ii) comparing said obtained expression profile to a reference profile to determine the deregulation status of multiple pathways for said subject.

20. A method of reducing side effects from the administration of two or more agents to a subject afflicted with cancer, said method comprising:
(a) determining a cancer subtype for said subject by:
(i) obtaining an expression profile from a sample from said subject; and
(ii) comparing said obtained expression profile to a reference profile to determine the deregulation status of multiple pathways for said subject;
(b) determining ineffective treatment protocols based on said determined cancer subtype; and
(c) reducing side effects by not treating said subject with said ineffective treatment protocols.

21. A method of generating an expression signature for a deregulated pathway, said method comprising:
(a) overexpressing an oncogene in a cell line to deregulate a pathway;
(b) determining an expression profile of multiple genes in the cell line; and
(c) comparing said obtained expression profile to a reference profile to determine an expression signature for a deregulated pathway.

22. The method of claim 21, wherein overexpressing an oncogene comprises transfecting the cell line with the oncogene.

23. The method of claim 21, wherein the expression profile is obtained by the use of a microarray.

24. The method of claim 21, wherein the expression profile comprises ten or more genes.

25. A method of generating an expression signature for a deregulated pathway, said method comprising:
(a) underexpressing a tumor suppressor in a cell line to deregulate a pathway;
(b) determining an expression profile of multiple genes in the cell line; and
(c) comparing said obtained expression profile to a reference profile to determine an expression signature for a deregulated pathway.

26. The method of claim 25, wherein underexpressing a tumor suppressor comprises targeted gene knockdown or knockout of the tumor suppressor in a cell line.

27. The method of claim 25, wherein the expression profile is obtained by the use of a microarray.

28. The method of claim 25, wherein the expression profile comprises ten or more genes.
